SEQUENCE LISTING (37 sequences)

SEQ ID NO: 1 - protein, 805 aa, Artificial Sequence [sequence omitted]

SEQ ID NO: 2 - protein, 723 aa, Artificial Sequence
  Isolated SARS-CoC-2 binding proetin complex
  MISC_FEATURE (1)..(723)
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVS

SEQ ID NO: 3 - protein, 598 aa, Artificial Sequence
  UniProtKB ID Q9BYF1-1
  MISC_FEATURE (1)..(598)
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYAD

SEQ ID NO: 4 - protein, 376 aa, Artificial Sequence
  UniProtKB ID Q9BYF1-1
  MISC_FEATURE (1)..(376)
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLR

SEQ ID NO: 5 - protein, 31 aa, Artificial Sequence
  UniProtKB ID Q9BYF1-1
  MISC_FEATURE (1)..(31)
QSTIEEQAKTFLDKFNHEAEDLFYQSSLASW

SEQ ID NO: 6 - protein, 29 aa, Artificial Sequence
  A Helix Forming Peptide
  MISC_FEATURE (1)..(29)
TEENVQNMNNAGDKWSAFLKEQSTLAQMY

SEQ ID NO: 7 - protein, 31 aa, Artificial Sequence
  A Helix Forming Peptide
  MISC_FEATURE (1)..(31)
EEQAKTFLDKFNHEAEDLFYQSSLASWNYNT

SEQ ID NO: 8 - protein, 10 aa, Artificial Sequence
  A B-turn peptide
  MISC_FEATURE (1)..(10)
AWDLGKGDFR

SEQ ID NO: 9 - protein, 19 aa, Artificial Sequence
  A Fragment of a Helix Forming Peptide
  MISC_FEATURE (1)..(19)
AGDKWSAFLKEQSTLAQMY

SEQ ID NO: 10 - protein, 23 aa, Artificial Sequence
  the fragment of a helix forming peptide
  MISC_FEATURE (1)..(23)
EEQAKTFLDKFNHEAEDLFYQSS

SEQ ID NO: 11 - protein, 8 aa, Artificial Sequence
  the fragment of a B-turn peptide
  MISC_FEATURE (1)..(8)
DLGKGDFR

SEQ ID NO: 12 - protein, 967 aa, Artificial Sequence
  Amino Acid ACE2 Signal
  MISC_FEATURE (1)..(967)
MSSSSWLLLSLVAVTAAQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSDKTHTCPPCPAPELLGGPSVFLFPPKPKDTLMISRTPEVTCVVVAVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 842 aa, Artificial Sequence
  Ace2 Signal Sequence
  MISC_FEATURE (1)..(842)
MSSSSWLLLSLVAVTAAQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADDKTHTCPPCPAPELLGGPSVFLFPPKPKDTLMISRTPEVTCVVVAVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 842 aa, Artificial Sequence
  ACE2 signal sequence
  MISC_FEATURE (1)..(842)
MSSSSWLLLSLVAVTAAQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHNEMGNIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADDKTHTCPPCPAPELLGGPSVFLFPPKPKDTLMISRTPEVTCVVVAVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - protein, 842 aa, Artificial Sequence
  ACE2 Signal Sequence
  MISC_FEATURE (1)..(842)
MSSSSWLLLSLVAVTAAQPTIEEQARAFLDKFNHEAEDLFYQSSLASWNYNTNITEENVQNMNNAGDKWSAFLKEQSTLAQMYPLQEIQILTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHNEMGRIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADDKTHTCPPCPAPELLGGPSVFLFPPKPKDTLMISRTPEVTCVVVAVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 842 aa, Artificial Sequence
  ACE2 Signal Sequence
  MISC_FEATURE (1)..(842)
MSSSSWLLLSLVAVTAAQPTIEEQARAFLDKFNHEAEDLFYQSSLASWNYNTNITEENVQNMNNAGDKWSAFLKEQSTLAQMYPLQEIQTLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHNEMGRIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADDKTHTCPPCPAPELLGGPSVFLFPPKPKDTLMISRTPEVTCVVVAVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - protein, 842 aa, Artificial Sequence
  ACE2 signal sequence
  MISC_FEATURE (1)..(842)
MSSSSWLLLSLVAVTAAQPTIEEQARAFLDKFNHEAEDLFYQSSLASWNYNTNITEENVQNMNNAGDKWSAFLKEQSTLAQMYPLQEIQNLIVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHNEMGRIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADDKTHTCPPCPAPELLGGPSVFLFPPKPKDTLMISRTPEVTCVVVAVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - protein, 842 aa, Artificial Sequence
  ACE2 signal sequence
  MISC_FEATURE (1)..(842)
MSSSSWLLLSLVAVTAAQPTIEEQAKAFLDKFNHEAEDLFYQSSLASWNYNTNITEENVQNMNNAGDKWSAFLKEQSTLAQMYPLQEIQILTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHNEMGNIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADDKTHTCPPCPAPELLGGPSVFLFPPKPKDTLMISRTPEVTCVVVAVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - protein, 842 aa, Artificial Sequence
  ACE2 signal sequence
  MISC_FEATURE (1)..(842)
MSSSSWLLLSLVAVTAAQPTIEEQAKAFLDKFNHEAEDLFYQSSLASWNYNTNITEENVQNMNNAGDKWSAFLKEQSTLAQMYPLQEIQTLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHNEMGNIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADDKTHTCPPCPAPELLGGPSVFLFPPKPKDTLMISRTPEVTCVVVAVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - protein, 307 aa, Artificial Sequence
  ACE2 signal sequence
  MISC_FEATURE (1)..(307)
GTEENVQNMNNAGDKWSAFLKEQSTLAQMYGGEEQAKTFLDKFNHEAEDLFYQSSLASWNYNTGGGGSGGAWDLGKGDFRDKTHTCPPCPAPELLGGPSVFLFPPKPKDTLMISRTPEVTCVVVAVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - protein, 326 aa, Artificial Sequence
  SS-HHB-Fc DANG hybrid protein
  MISC_FEATURE (1)..(326)
MDWTWRFLFVVAAATGVQSGTEENVQNMNNAGDKWSAFLKEQSTLAQMYGGEEQAKTFLDKFNHEAEDLFYQSSLASWNYNTGGGGSGGAWDLGKGDFRDKTHTCPPCPAPELLGGPSVFLFPPKPKDTLMISRTPEVTCVVVAVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 22 - protein, 281 aa, Artificial Sequence
  minHHB-Fc DANG hybrid protein
  MISC_FEATURE (1)..(281)
GAGDKWSAFLKEQSTLAQMYGGEEQAKTFLDKFNHEAEDLFYQSSGDLGKGDFRDKTHTCPPCPAPELLGGPSVFLFPPKPKDTLMISRTPEVTCVVVAVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 23 - protein, 300 aa, Artificial Sequence
  SS-minHHB-Fc DANG hybrid protein
  MISC_FEATURE (1)..(300)
MDWTWRFLFVVAAATGVQSGAGDKWSAFLKEQSTLAQMYGGEEQAKTFLDKFNHEAEDLFYQSSGDLGKGDFRDKTHTCPPCPAPELLGGPSVFLFPPKPKDTLMISRTPEVTCVVVAVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 24 - protein, 262 aa, Artificial Sequence
  the minHB-Fc DANG hybrid protein
  MISC_FEATURE (1)..(262)
GEEQAKTFLDKFNHEAEDLFYQSSGAWDLGKGDFRDKTHTCPPCPAPELLGGPSVFLFPPKPKDTLMISRTPEVTCVVVAVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 25 - protein, 281 aa, Artificial Sequence
  minHB-Fc DANG hybrid protein
  MISC_FEATURE (1)..(281)
MDWTWRFLFVVAAATGVQSGEEQAKTFLDKFNHEAEDLFYQSSGAWDLGKGDFRDKTHTCPPCPAPELLGGPSVFLFPPKPKDTLMISRTPEVTCVVVAVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 26 - protein, 17 aa, Artificial Sequence
  Example of the signal sequence
  MISC_FEATURE (1)..(17)
MSSSSWLLLSLVAVTAA

SEQ ID NO: 27 - protein, 19 aa, Artificial Sequence
  Example of the signal sequence
  MISC_FEATURE (1)..(19)
MDWTWRFLFVVAAATGVQS

SEQ ID NO: 28 - protein, 19 aa, Artificial Sequence
  Example of the signal sequence
  MISC_FEATURE (1)..(13)
MEFGLSWVFLVALFRGVQS

SEQ ID NO: 29 - protein, 19 aa, Artificial Sequence
  Example of the signal sequence
  MISC_FEATURE (1)..(19)
MELGLSWIFLLAILKGVQC

SEQ ID NO: 30 - protein, 19 aa, Artificial Sequence
  Example of the signal sequence
  MISC_FEATURE (1)..(19)
MELGLRWVFLVAILEGVQC

SEQ ID NO: 31 - protein, 19 aa, Artificial Sequence
  Example of the signal sequence
  MISC_FEATURE (1)..(19)
MKHLWFFLLLVAAPRWVLS

SEQ ID NO: 32 - protein, 19 aa, Artificial Sequence
  Example of the signal sequence
  MISC_FEATURE (1)..(19)
MDWTWRILFLVAAATGAHS

SEQ ID NO: 33 - protein, 19 aa, Artificial Sequence
  Example of the signal sequence
  MISC_FEATURE (1)..(19)
MEFGLSWLFLVAILKGVQC

SEQ ID NO: 34 - protein, 19 aa, Artificial Sequence
  Example of the signal sequence
  MISC_FEATURE (1)..(19)
MEFGLSWVFLVALFRGVQC

SEQ ID NO: 35 - protein, 26 aa, Artificial Sequence
  Example of the signal sequence
  MISC_FEATURE (1)..(26)
MDLLHKNMKHLWFFLLLVAAPRWVLS

SEQ ID NO: 36 - protein, 22 aa, Artificial Sequence
  Example of the signal sequence
  MISC_FEATURE (1)..(22)
MDMRVPAQLLGLLLLWLSGARC

SEQ ID NO: 37 - protein, 22 aa, Artificial Sequence
  Example of the signal sequence
  MISC_FEATURE (1)..(22)
MKYLLPTAAAGLLLLAAQPAMA

(19) United States
(12) Patent Application Publication
(10) Pub. No.: US 2023/0203466 A1
(43) Pub. Date: Jun. 29, 2023

(54) ACE2 RECEPTOR POLYMORPHISMS AND VARYING SUSCEPTIBILITY TO SARS-COV-2, METHODS FOR DIAGNOSIS AND TREATMENT

(71) Applicant: MedGenome Inc., Foster City, CA (US)

(72) Inventors: Somasekar Seshagiri, Foster City, CA (US); Eric Stawiski, Foster City, CA (US); Kushai Suryamohan, Foster City, CA (US); Ravi Gupta, Foster City, CA (US); Jagath Reddy Junutula, Foster City, CA (US)

(21) Appl. No.: 17/916,723

(22) PCT Filed: Apr. 5, 2021
(86) PCT No.: PCT/US21/25824

§ 371 Date: Oct. 3, 2022

Related U.S. Application Data
(60) Provisional application No. 63/005,163, filed on Apr. 3, 2020.
(60) Provisional application No. 63/019,952, filed on May 4, 2020.

(51) Int. Cl. C12N 9/48; C07K 16/10; G01N 33/569

(52) U.S. Cl. CPC C12N 9/485; C07K 16/10; C12Y 304/17023; G01N 33/56983; C07K 2319/33; C07K 2319/30; C07K 2317/622; C07K 2317/55

(57) ABSTRACT

Human ACE2 variants are provided including methods of use thereof. The ACE2 receptor variants may be used for diagnosis and treatment of COVID-19.

This subject patent application claims the benefit under 35 U.S.C. § 119(e) to U.S. Provisional Application No. 63/005,163, filed Apr. 3, 2020, and U.S. Provisional Application No. 63/019,952, filed May 4, 2020, the contents of which are herein incorporated by reference in their entireties into the present patent application for all purposes.

Throughout this application various publications are referenced. All publications, gene transcript identifiers, patents, and patent applications mentioned in this specification are herein incorporated by reference to the same extent as if each individual publication, gene transcript identifiers, patent, or patent application was specifically and individually indicated to be incorporated by reference.

### BACKGROUND GF THE INVENTION

Coronaviruses (CoVs) are widely distributed in nature and pose a serious threat to humans and a range of mammalian hosts, causing respiratory, gastrointestinal, and central nervous system diseases (Li, 2016). CoVs are enveloped non-segmented positive-sense single stranded RNA viruses and are classified into α-, β-, γ-, and δ-CoVs (Li, 2016). While α- and β-CoVs infect mammals, the γ- and δ-CoVs generally infect birds (Li, 2016). Previously, α-CoVs HCoV-229E and HCoV-NL63, and β-CoVs HCoV-HKU1 and HCoV-OC43 have been found to infect humans leading to mild symptoms (Graham and Baric, 2010; Li, 2016). More recently, three β-CoVs: severe acute respiratory syndrome coronavirus (SARS-CoV) in 2003 (Holmes, 2003; Li, 2016), Middle-East respiratory syndrome coronavirus in 2012 (MERS-CoV) (Li, 2016; Zaki et al., 2012), and more recently SARS-CoV-2 in 2019 (Chan et al., 2020a; Huang et al., 2020; Zhu et al., 2020) have crossed the species barrier to infect humans resulting in respiratory illnesses including pneumonia that can be fatal.

SARS-CoV-2 is a novel coronavirus (2019-nCoV) first reported in December 2019 and is the cause of an ongoing global pandemic (Chan et al., 2020a; Huang et al., 2020; Zhu et al., 2020). It has infected over 39 million people in 181 countries leading to over 1.2 million deaths as of Oct. 19, 2020 (JHU, 2020). Sequence analysis of the SARS-CoV-2 genome revealed that it is closer to the bat CoV RaTG13 (96.2% identical) than to SARS-CoV (79.5% identical) that was responsible for the 2003 epidemic, suggesting that this novel virus originated in bats independently before jumping to humans either directly or through a yet to be determined intermediary host (Guo et al., 2020).

Severe acute respiratory syndrome coronavirus 2 (SARS-CoV-2) is the cause of coronavirus disease (COVID-19) that has resulted in a global pandemic. It is a highly contagious positive strand RNA virus and its clinical presentation includes severe to critical respiratory disease that appears to be fatal in ˜3-5% of the cases. The viral spike (S) coat protein engages the human angiotensin-converting enzyme 2 (ACE2) cell surface protein to invade the host cell. The SARS-CoV-2 S-protein has acquired mutations that increase its affinity to human ACE2 by ˜10-15-fold compared to SARS-CoV S-protein, making it highly infectious. In this study, we assessed if ACE2 polymorphisms might alter host susceptibility to SARS-CoV-2 by affecting the ACE2 S-protein interaction. Our comprehensive analysis of several large genomic datasets that included over 290,000 samples representing >400 population groups identified multiple ACE2 protein-altering variants, some of which mapped to the S-protein-interacting surface. Using recently reported structural data and a recent S-protein-interacting synthetic mutant map of ACE2, we have identified natural ACE2 variants that are predicted to alter the virus-host interaction and thereby potentially alter host susceptibility. In particular, human ACE2 variants S19P, I21V, E23K, K26R, T27A, N64K, T92I, Q102P and H378R are predicted to increase susceptibility. The T92I variant, part of a consensus NxT/S N-glycosylation motif, confirmed the role of N90 glycosylation in providing some protection against non-human CoVs. Other ACE2 variants K31R, N33I, H34R, E35K, E37K, D38V, Y50F, N51S, M62V, K68E, F72V, Y83H, G326E, G352V, D355N, Q388L and D509Y are putative protective variants predicted to show decreased binding to SARS-CoV-2 S-protein.

Using biochemical assays, we found that while K31R and E37K had a decreased affinity, K26R and T92I variants had an increased affinity for SARS-CoV-2 S-protein when compared to wildtype ACE2, confirming our structural predictions. Consistent with this, soluble ACE2 K26R and T92I were more effective in blocking entry of S-protein pseudotyped virus. These data suggest that ACE2 variants can modulate the susceptibility to SARS-CoV-2.

As with SARS-CoV and a related alphacoronaviruses NL63 (HCoV-NL63), the SARS-CoV-2 employs the human angiotensin-converting enzyme 2 (ACE2) cell surface protein as a receptor to gain entry into the cells (Hoffmann et al., 2020; Letko et al., 2020; Lin et al., 2008; Wan et al., 2020; Zhou et al., 2020). The virus surface spike glycoprotein (S-protein) constitutes a key determinant of viral host range and contains two domains, S1 and S2, which are separated by a protease cleavage site (Li, 2016). A successful host cell invasion by the virus involves direct binding of the virus S1 receptor binding domain (RBD) to the host ACE2 peptidase extracellular domain (PD), exposing the S1-S2 inter-domain protease site that upon cleavage by host proteases, leads to S2-mediated virus-host cell membrane fusion (Belouzard et al., 2009; Hoffmann et al., 2020; Li, 2016; Li et al., 2005a; Simmons et al., 2005).

The receptor binding domain (RBD) within S1 binds directly to the peptidase domain (PD) of ACE2, while S2 mediates membrane fusion (Li, 2016; Li et al., 2005a; Simmons et al., 2005). As the S1 subunit binds the host ACE2, an exposed protease site on S2 is cleaved by host proteases facilitating membrane fusion and viral infection (Belouzard et al., 2009; Simmons et al., 2005).

The SARS-CoV-2 S-protein is 98% identical to the bat CoV RaTG13 S-protein, with the exception of an insertion that is also absent in the SARS-CoV S-protein in the S1/S2 inter-domain protease cleavage site. This difference has been proposed to alter SARS-CoV-2 tropism and enhance its transmissibility (Walls et al., 2020).

Several structural studies involving the SARS-CoV-2 S-protein RBD and ACE2 peptidase domain (PD) have identified the key residues involved in their interaction (Shang et al., 2020; Walls et al., 2020; Wrapp et al., 2020; Yan et al., 2020). The S-protein RBD was reported to bind ACE2 PD with ˜10- to 20-fold higher affinity (˜15 nM) when compared to the SARS-CoV S-protein RBD (Shang et al., 2020; Wrapp et al., 2020), potentially contributing to the high rate of SARS-CoV-2 infection.

As the interactions between the ACE2 receptor and S-protein RBD interface are critical for the cellular entry of the virus, we wanted to ascertain if there were natural ACE2 variations that would decrease or increase its affinity to the S-protein RBD and may thus protect or render individuals more susceptible to the virus. Consistent with this possibility, a saturation mutagenesis screen of select ACE2 PD residues identified variants that showed enhanced or decreased binding to S-protein (Chan et al., 2020b).

Since COVID-19 poses a serious threat to animals and humans, it is important to be able to identify it accurately and quickly to reduce COVID-19's deleterious health and economic impact. We have analyzed the ACE2 protein altering variants in a large number of data set populations and identified polymorphisms that will likely either protect or render them more susceptible to the virus.

We have addressed this need by discovering rationally designed, catalytically inactive, human ACE2 that carries one or more of the natural variants to obtain improved binding to SARS viral S-protein RBD that can be developed as a soluble protein with or without an Fc domain for treatment of COVID-19. Such a recombinant ACE2 protein can be engineered to create a pan-CoV neutralizing drug that is broad and can neutralize CoVs that may emerge during future epidemics.

In this study, we have analyzed ACE2 protein-altering variants in a large cohort of human population groups and identified polymorphisms that either likely protect or render individuals more susceptible to the virus. Understanding these changes at the molecular level, combined with the genotype and epidemiological data will allow the elucidation of population risk profiles and also help advance therapeutics such as a rationally designed soluble ACE2 decoy-receptor for treatment of COVID-19.

### SUMMARY OF THE INVENTION

Isolated SARS-CoV-2 binding protein complexes comprising ACE2 receptor variations and variants which may predict resistance and sensitivity to a SARS coronavirus, COVID-19 are provided, which proteins comprise sequence modification that enhance the stability and/or utility of the protein. Human ACE2 receptor variations and variants are preferred. The ACE2 receptor variants may be used for diagnosis and treatment of COVID-19.

The invention also provides methods for monitoring the course of SARS-CoV-2 infection in a subject. In one embodiment, the method comprises obtaining a sample from the subject, determining amino acid sequence of ACE2 of the subject, comparing identity of amino acid so determined to reference amino acids known to affect SARS-CoV-2 interaction with ACE2, wherein finding an amino acid change favoring interaction with surface spike glycoprotein, S protein, of SARS-CoV-2 are any of S19P, I21T/V, E23K, A25T, K26E or K26R, T27A, F40L, Q60R, N64K, W69C, T92I, Q102P, Q325R, M366T, D367V, H374R, H378R, M383T, E398D, E398K, T445M, I446M, and Y510H, and wherein an amino acid change resulting in less favorable interaction with S protein of SARS-CoV-2 are any of K31R, N33I, H34R, E35K, E37K, D38V, Y50F, N51D or N51S, M62I or M62V, A65S, K68E, F72H, M82I, Y83H, P84T, V93G, N290H, G326E, E329G, P346S, G352V, D355N, T371K, Q388L, P389H, F504I or F504L, H505R, D509Y, S511P, R514G, Y515C and R518T and predicting a subject to have a more severe course of infection for the subject with an amino acid change favoring interaction with S protein of SARS-CoV-2 or a milder course of infection for the subject with an amino acid change resulting in less favorable interaction with S protein of SARS-CoV-2.

The invention also provides methods for assessing risk of being infected by SARS-CoV-2 virus in a subject. In one embodiment, the method comprises obtaining a sample from the subject, determining amino acid sequence of ACE2 of the subject, comparing identity of amino acid so determined to reference amino acids known to affect SARS-CoV-2 interaction with ACE2, wherein finding an amino acid change resulting in increased risk of being infected are any of S19P, I2T/V, E23K, A25T, K26E or K26R, T27A, F40L, Q60R, N64K, W69C, T92I, Q102P, Q325R, M366T, D367V, H374R, H378R, M383T, E398D, E398K, T445M, 1446M, and Y510H, and wherein an amino acid change resulting in decreased risk of being infect are any of K31R, N33I, H34R, E35K, E37K, D38V, Y50F, N51D or N51S, M62I or M62V, A65S, K68E, F72H, M82I, Y83H, P84T, V93G, N290H, G326E, E329G, P346S, G352V, D355N, T371K, Q388L, P389H, F504I or F504L, H505R, D509Y, S511P, R514G, Y515C and R518T, and predicting a subject to have an increased or decreased risk based on finding a match falling into the two groups.

The invention also provides kits for assessing risk or course of a SARS-CoV-2. In one embodiment, the kit comprises oligonucleotide or nucleic acid fragment for assessing polymorphism of ACE2 gene and instruction for use. In a further embodiment, the polymorphism is directed to the coding region of the ACE2 gene. In another embodiment, the polymorphism is directed to the SARS-CoV-2 S protein interaction site on ACE2 protein as provided in FIG. 18. In an additional embodiment, the oligonucleotide or nucleic acid fragment is used to assess the status of the first 115 codons of ACE2 gene.

The invention also provides kits for detecting COVID-19 comprising an ACE2 variant from any of the Tables herein and an informational insert.

### BRIEF DESCRIPTION OF THE DRAWINGS

FIG. 1a-d. ACE2 polymorphisms. a. Pie chart representing protein altering variations in ACE2 by allele count and source. b. Log base 10 pseudo count adjusted (+1) observed ACE2 allele counts of mutants predicted to impact S-protein binding. Singletons are marked with a \hat{ } and direct S-protein contact residues are underlined. c. ACE2 protein domain showing positions with polymorphisms that can alter SARS-CoV-2 S-protein binding. Recurrent polymorphisms (n>1) that were predicted to not impact S-protein binding are shown in light grey. Residues within the ACE2 PD known to interact with viral S-protein are shown as red vertical lines within the peptidase domain in the ACE2 diagram. d. Multiple sequence alignment of the S-protein interacting ACE2 sequence from indicated species. ACE2 NxT/S glycosylation motif disrupted in dog, rat, palm civet and several bat ACE2 is highlighted in red (darker gray rectangular boxes under ACE2 amino acid residues at position 90 to 92 for example dog, mouse, chicken, zebrafish, frog, etc.). ACE2 residues that mediate contact with NL63-CoV, SARS-CoV and SARS-CoV-2 are shown as blue (top; darker gray), green (middle; light gray) and orange (bottom; black) bars, respectively.

FIG. 2a-b. Genetic variation of human ACE2 gene. (a) Fst index of exonic variants of ACE2, calculated from 57,783 female individuals across eight populations in gnomAD. Canonical transcript of ACE2 (ENST00000427411) and two Pfam domains are shown along with the positions of known SARS-CoV-2 contact residues. Peptidase domain harbor variants with lower variation (Wilcox p=0.0656). (b) ACE2 is highly constrained (pLI=0.9977), with the observed-to-expected ratio of the number of pLoF variants of 0.0968, consistent with the constrained genes (highlighted in cyan). Note clustering of cyan dots between “0.0” Observed/Expected Ratio for “Other Genes” and dash line, inclusive.

FIG. 3a-b. ACE2 sequence comparison. (a). Phylogenetic tree of ACE2 sequences from selected species, (b) Multiple sequence alignment of representative primate ACE2 sequences and ACE2 sequences of putative natural and intermediate reservoirs of coronaviruses. Pink boxes highlight species (small rectangular darker gray boxes under ACE2 amino acid residues at position 90 to 92 for common vampire bat, pale spear-nosed bat, least horseshoe bat and Japanese house bat) where the canonical NxT/S motif is absent or altered.

FIG. 4. A schematic diagram of a full-length human ACE2 protein and the sequence thereof (UniProtKB ID: Q9BYF1-1).

FIG. 5a-e. A schematic diagram of IgG-ACE2 fusion proteins including a human ACE2 full-length extra cellular domain (ecd) or a truncated ecd.

FIG. 6a-c. A schematic diagram of Fc-ACE2 fusion proteins.

FIG. 7a-h. A schematic diagram of hACE2 therapeutic variants and their sequences.

FIG. 8. A schematic diagram of an HHB (helix2-helix1-beta turn), a novel truncated ACE2 therapeutic agent.

FIG. 9. An amino acid sequence of a minHHB, a novel truncated ACE2 therapeutic agent.

FIG. 10. A schematic diagram of an HB (helix1-beta turn), a novel truncated ACE2 therapeutic and a sequence thereof.

FIG. 11. A schematic diagram of an ACE2ecd-Fc-scFv, a bi-specific fusion protein and a sequence thereof.

FIG. 12. Bi-specific knob-hole format ACE2ecd-anti-SARS-CoV2-S antibody.

FIG. 13. hACE2ecd-Fc fusion proteins.

FIG. 14A-B. COVID-19 diagnostic assays utilizing enhanced hACE2-Fc variant in an ELISA format. FIG. 14A: ELISA test for detecting CoV2-virus from the patient samples (e.g., blood/serum/saliva samples). Human ACE2-Fc fusion protein consisting of any one of N33I, A80G and T92I mutations or their combinations are coated to ELISA plate at 1 ug/mL. Alternatively, the human ACE2-Fc fusion protein consisting of any one of S19P, K26R, K26E, T27A, K3 IR, N33I, H34R, E35K, E35D. E37K, D38V, A80G, M82I, Y83H, N90E, N90T, T92I, Q325E, G326E, E329G, D355N and P389H mutations or their combinations are coated to ELISA plate at 1 μg/mL. In a preferred embodiment, the human ACE2-Fc fusion protein comprises mutations selected from the group consisting of S19P-K26R. S19P-N90E, S19P-T92I, K26R-N90E, K26R-T92I, S19P-K26R-N90E and S19P-K26R-N92I is a preferred ACE2 mutants uses for therapeutic or diagnostic purposes. Bound virus or viral-spike protein is detected with biotinylated non-competing anti-spike protein antibody (for example CR3022) and streptavidin-HRP. FIG. 14B: ELISA test for detecting anti-CoV2-virus antibodies (IgG, IgA or IgM) in the patient samples (e.g., blood/serum/saliva samples). S-protein or N-protein are coated to ELISA plate at 1 ug/mL. Bound anti-virus antibodies in the patient blood/serum/saliva are detected using goat anti-human IgG/IgA/IgM-HRP.

FIG. 15. Example of use of a SARS-CoV-2 binding protein of the invention in a lateral flow diagnostic antibody assay to detect SARS-CoV-2 virus or SARS-CoV-2 S-protein.

FIG. 16. A schematic diagram of a rapid method for detection of SARS-CoV-2.

FIG. 17. Amino acid sequences of two bi-specific scFv's designated ACE2ecd(1-615)-(T92I)-H374N-H378N-Fc-(DANG)-3B11scFv and DPP4ecd(39-766)-S630A-Fc-(DANG)-CR3022scFv. Note that N-terminal human ACE2 signal peptide sequence (amino acid residue 1-17 of human ACE2 protein; dark shaded region at the beginning of each sequence) is covalently linked to ACE2ecd variant (amino acid residue 18-615; T92I glycosylation-deficient mutation and I374N-H378N peptidase-deficient mutations; no shading) or DPP4ecd variant (amino acid residues 39-766; S630A mutation; no shading), which is in turn covalently linked to an IgG Fc fragment (lighter shading) with DANG effector (D265A and N297G) mutation (in bold letter A or G in the lighter shaded region), and scFV for either 3B11 scFv or CR3022 scFv at the C-terminus of the fusion protein, respectively. The darker shaded glycine-serine rich sequence are linkers between the Fe fragment and scFv and between the light and heavy variable domains of scFv. CR3022 scFv binds to RBD of SARS-CoV-2 without blocking the binding of RBD of SARS-CoV-2 to ACE2 (PDB: 6W41).

FIG. 18a-b. Polymorphisms identified in human ACE2 mapped to the structure of human ACE2 in complex with the SARS-CoV-2 RBD. Residues in ACE2 showing polymorphic variation in human population were mapped on to the structure of the ACE2/SARS-CoV-2 RBD (PDB: 6VW1) and colored according to their effect on the predicted affinity between human ACE2. Polymorphisms that were predicted to enhance the binding between ACE2 and the S-protein are colored in magenta (enhancing variant indicated by “*” sign). Polymorphisms that are predicted to disrupt the binding between ACE2 and the S-protein are colored in dark blue (disruptive variant indicated by “+” sign). The variable loop in the ridge binding motif consisting of residues V483 and E484 is shown in red. Region in the structure (PDB: 6LZG) zoomed-in to show variants predicted to enhance or disrupt the ACE2-SARS-CoV-2 interaction.

FIG. 19A-C. Binding affinity of SARS-CoV-2 S-RBD, S1 and S-trimer. ELISA assay measuring the affinity of indicate ACE2 WT or variants for SARS-CoV-2 S-RBD (a), S1 subunit (b) and S-trimer (c).

FIG. 20. Binding affinity of SARS-CoV-2 S-RBD. ELISA assay measuring the affinity of human ACE2 WT or variants for SARS-CoV-2 S-RBD.

FIG. 21. Binding affinity of SARS-CoV-2 S1. ELISA assay measuring the affinity of human ACE2 WT or variants for SARS-CoV-2 S1 subunit.

FIG. 22. Binding affinity of SARS-CoV-2 S-trimer. ELISA assay measuring the affinity of human ACE2 WT or variants for SARS-CoV-2 S-trimer.

FIG. 23. Lollipop plot of ACE2 protein showing protein altering polymorphic variants observed across the entire protein. Allele counts for each polymorphism is shown inside or above each circle. Empty circles indicate singletons.

FIG. 24a-c. Genealogical estimation of variant age (GEVA) analysis of variants in a 1 Mb region around the ACE2 gene; colors distinguish non-coding (gray), synonymous (blue), and missense (red) variants, predicted using the Ensembl Variant Effect Predictor (VEP) analysis. (a) Physical location (position on Chromosome X) and estimated age of the variants dated using GEVA; gene tracts (top) indicate the location of the larger genes within the region, highlighting the ACE2 gene (shaded area). (b) Comparison between allele frequency (count of the derived allele in the sample) and estimated age; highlighting variants within (or VEP predicted effects on) the ACE2 gene (black circles). (c) Empirical cumulative distribution of variants by estimated age, comparing variants outside the ACE2 gene region (solid lines) to variants affecting ACE2 (dashed lines).

FIG. 25a-c. Purified recombinant S-protein and ACE2 were resolved on 4-15% SDS-PAGE (Mini-PROTEAN TGX Stain-Free Precast Gel).

FIG. 26. Heatmap showing human ACE2 polymorphism that map to the ACE2-RBD interaction region and the corresponding enrichment/depletion scores from a recent study (Science 2020, 10.1126/science.abc0870).

### DETAILED DESCRIPTION OF THE INVENTION

### Definitions

It is to be understood that this invention is not limited to the particular methodology, protocols, cell lines, animal species or genera, and reagents described, as such may vary. It is also to be understood that the terminology used herein is for the purpose of describing particular embodiments only, and is not intended to limit the scope of the present invention, which will be limited only by the appended claims.

As used herein the singular forms “a”, “and”, and “the” include plural referents unless the context clearly dictates otherwise. Thus, for example, reference to “a cell” includes a plurality of such cells and reference to “the culture” includes reference to one or more cultures and equivalents thereof known to those skilled in the art, and so forth. All technical and scientific terms used herein have the same meaning as commonly understood to one of ordinary skill in the art to which this invention belongs unless clearly indicated otherwise.

As used herein, the terms “purified” and “isolated” when used in the context of a polypeptide that is substantially free of contaminating materials from the material from which it was obtained, e.g. cellular materials, such as but not limited to cell debris, cell wall materials, membranes, organelles, the bulk of the nucleic acids, carbohydrates, proteins, and/or lipids present in cells. Thus, a polypeptide that is isolated includes preparations of a polypeptide having less than about 30%, 20%, 10%, 5%, 2%, or 1% (by dry weight) of cellular materials and/or contaminating materials. As used herein, the terms “purified” and “isolated” when used in the context of a polypeptide that is chemically synthesized refers to a polypeptide which is substantially free of chemical precursors or other chemicals which are involved in the syntheses of the polypeptide.

The term “polypeptide,” “peptide,” “oligopeptide,” and “protein,” are used interchangeably herein, and refer to a polymeric form of amino acids of any length, which can include coded and non-coded amino acids, chemically, or biochemically modified or derivatized amino acids, and polypeptides having modified peptide backbones.

The polypeptides may be isolated and purified in accordance with conventional methods of recombinant synthesis. Exemplary coding sequences are provided, however one of skill in the art can readily design a suitable coding sequence based on the provided amino acid sequences. Methods which are well known to those skilled in the art can be used to construct expression vectors containing coding sequences and appropriate transcriptional/translational control signals. These methods include, for example, in vitro recombinant DNA techniques, synthetic techniques and in vivo recombination/genetic recombination. Alternatively, RNA capable of encoding the polypeptides of interest may be chemically synthesized. One of skill in the art can readily utilize well-known codon usage tables and synthetic methods to provide a suitable coding sequence for any of the polypeptides of the invention. The nucleic acids may be isolated and obtained in substantial purity. Usually, the nucleic acids, either as DNA or RNA, will be obtained substantially free of other naturally-occurring nucleic acid sequences, generally being at least about 50%, usually at least about 90% pure and are typically “recombinant,” e.g., flanked by one or more nucleotides with which it is not normally associated on a naturally occurring chromosome. The nucleic acids of the invention can be provided as a linear molecule or within a circular molecule, and can be provided within autonomously replicating molecules (vectors) or within molecules without replication sequences. Expression of the nucleic acids can be regulated by their own or by other regulatory sequences known in the art. The nucleic acids of the invention can be introduced into suitable host cells using a variety of techniques available in the art.

An “effective amount” or a “sufficient amount” of a substance is that amount sufficient to cause a desired biological effect, such as beneficial results, including clinical results, and, as such, an “effective amount” depends upon the context in which it is being applied. In the context of this invention, an example of an effective amount of a vaccine is an amount sufficient to induce an immune response (e.g., antibody production) in an individual. An effective amount can be administered in one or more administrations.

Folding, as used herein, refers to the process of forming the three-dimensional structure of polypeptides and proteins, where interactions between amino acid residues act to stabilize the structure. Non-covalent interactions are important in determining structure, and the effect of membrane contacts with the protein may be important for the correct structure. For naturally occurring proteins and polypeptides or derivatives and variants thereof, the result of proper folding is typically the arrangement that results in optimal biological activity, and can conveniently be monitored by assays for activity, e.g. ligand binding, enzymatic activity, etc.

In some instances, for example where the desired product is of synthetic origin, assays based on biological activity will be less meaningful. The proper folding of such molecules may be determined on the basis of physical properties, energetic considerations, modeling studies, and the like.

Separation procedures of interest include affinity chromatography. Affinity chromatography makes use of the highly specific binding sites usually present in biological macromolecules, separating molecules on their ability to bind a particular ligand. Covalent bonds attach the ligand to an insoluble, porous support medium in a manner that overtly presents the ligand to the protein sample, thereby using natural biospecific binding of one molecular species to separate and purify a second species from a mixture. Antibodies are commonly used in affinity chromatography. Preferably a microsphere or matrix is used as the support for affinity chromatography. Such supports are known in the art and are commercially available, and include activated supports that can be combined to the linker molecules. For example, Affi-Gel supports, based on agarose or polyacrylamide are low pressure gels suitable for most laboratory-scale purifications with a peristaltic pump or gravity flow elution. Affi-Prep supports, based on a pressure-stable macroporous polymer, are suitable for preparative and process scale applications.

Proteins may also be separated by ion exchange chromatography, and/or concentrated, filtered, dialyzed, etc., using methods known in the art. The methods of the present invention provide for proteins containing unnatural amino acids that have biological activity comparable to the native protein. One may determine the specific activity of a protein in a composition by determining the level of activity in a functional assay, quantitating the amount of protein present in a non-functional assay, e.g. immunostaining, ELISA, quantitation on coomassie or silver stained gel, etc., and determining the ratio of biologically active protein to total protein. Generally, the specific activity as thus defined will be at least about 5% that of the native protein, usually at least about 10% that of the native protein, and may be about 25%, about 50%, about 90% or greater.

### Compositions of the Invention

The invention provides SARS-CoV-2 binding protein complexes comprising ACE2 receptor variations and variants which may predict resistance and sensitivity to a SARS coronavirus, COVID-19. Human ACE2 receptor variations and variants are preferred. The ACE2 receptor variants may be used for diagnosis and treatment of COVID-19.

### Isolated SARS-CoV-2 Binding Protein Complexes

The invention also provides isolated SARS-CoV-2 binding protein complexes. As used herein, examples of a complex includes conjugates and fusion proteins. In one embodiment, the SARS-CoV-2 binding protein complex comprises an extracellular domain or fragment thereof of an angiotensin converting enzyme 2 (ACE2) protein or its variant joined to a non-ACE2 molecule or compound.

In accordance with the practice of the invention, the non-ACE2 compound may be a biological entity. Examples of suitable biological entities include, but are not limited to, proteins, polypeptide, peptides and albumin. The proteins may be serum proteins. The serum proteins may comprises any of antibody, serum albumin, beta-1-B-glycoprotein or Hemopexin (Hpx).

The protein may be an immunoglobulin molecule or antibody molecule or variant or fragment thereof. The antibody fragment may be a Fc. Examples of suitable antibody fragment include, but are not limited to, Fab, Fab′, F(ab)′, scFv, and F(ab)′₂. In a preferred embodiment, the antibody recognizes and binds a SARS-CoV-2. SARS-CoV-2 antibodies are known (ter Meulen J, van den Brink E N, Poon L L M, Marissen W E, Leung C S W, et al. (2006) Human monoclonal antibody combination against SARS coronavirus: Synergy and coverage of escape mutants. PLoS Med 3(7): e237. DOI: 10.1371/jourmal.pmed.0030237; Meng Yuan et al., Science 3 Apr. 2020: eabb7269, DOI: 10.1126/science.abb7269; Author links open overlay panel; ShiboJiang et al., Trends in Immunology, Volume 41, Issue 5, May 2020, Pages 355-359; Catalan-Dibene, J. Human antibodies can neutralize SARS-CoV-2. Nat Rev Immunol (2020). https://doi.org/10.1038/s41577-020-0313-6; Bin Ju, et al. Potent human neutralizing antibodies elicited by SARS-CoV-2 infection, bioRxiv 2020.03.21.990770; doi: https://doi.org/10.1101/2020.03.21.990770).

In another embodiment of the invention the non-ACE2 compound may be a chemical entity. Examples of suitable chemical entity include, but are not limited to, poly(ethylene glycol) (“PEG”). The PEG may be linear or branched. In one embodiment, the PEG has a molecular weight of from about 5,000 Daltons (5 kDa) to about 100,000 Daltons (100 kDa). In another embodiment, the PEG has a molecular weight of from about 10 kDa to about 60 kDa.

In one embodiment of the isolated SARS-CoV-2 binding protein complex, the ACE2 protein is derived from a mammal. Examples of mammals include, but are not limited to, mouse, rat, dog, cat, civet, pangolin, bat, pig, guinea pig, goat, sheep, donkey, horse, camel, chimpanzee, monkey, gorilla, cattle, and human. In a preferred embodiment of the invention, the mammal is human.

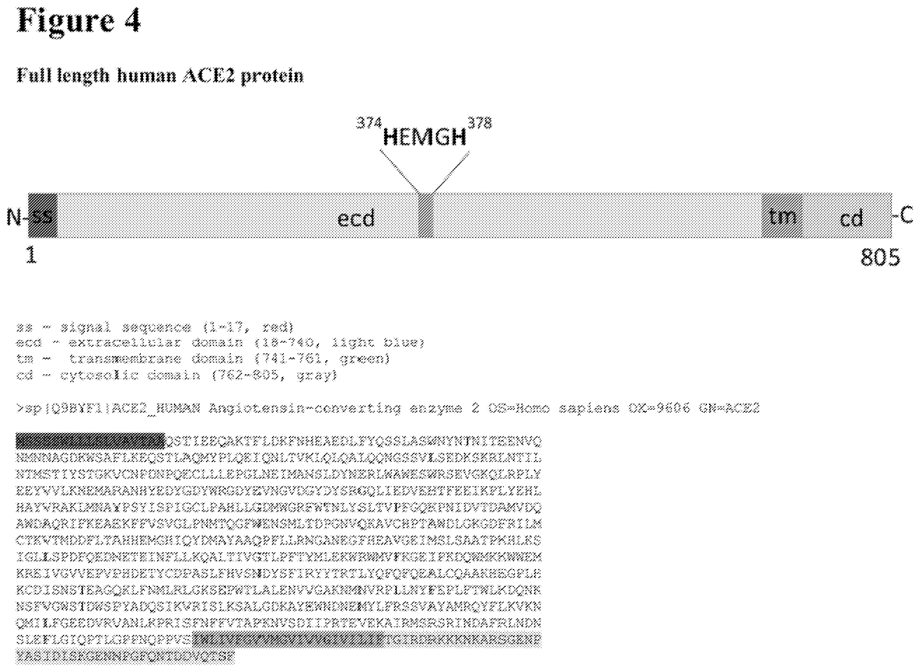

In one embodiment, the ACE2 protein may be a full length human ACE2 protein as shown in FIG. 4 or SEQ ID NO: 1 (UniProtKB ID: Q9BYF1-1):

(SEQ ID NO: 1)
MSSSSWLLLSLVAVTAAQSTIEEQAKTFLDKFNHEAEDLFYQSSLASWNY
NTNITEENVQNMNNAGDKWSAFLKEQSTLAQMYPLQEIQNLTVKLQLQAL
QQNGSSVLSEDKSKRINTILNTMSTIYSTGKVCNPDNPQECLLLEPGLNE
IMANSLDYNERLWAWESWRSEVGKQLRPLYEEYVVLKNEMARANHYEDYG
DYWRGDYEVNGVDGYDYSRGQLLEDVERTFEEIKPLYEHLHAYVRAKIMN
AYPSYISPIGCLPAALLGDMWGREWTNLYSLTVPFGQKPNIDVTDAMVDQ
AWDAQRIFKEAEKFFVSVGLPNMTQGFWENSMLTDPGNVQKAVCHPTAWD
LGKGDFRILMCTKVTMDDFLTAHHEMGHIQYDMAYAAQPFLLRNGANEGF
REAVGEIMSLSAATPKHLKSIGLLSPDFQEDNETEINFLLKQALTIVGTL
PFTYMLEKWRWMVFKGEIPKDQWMKKWWEMKREIVGVVEPVPHDETYCDP
ASLFHVSNDYSFIRYYTRTLYQFQFQEALCQAAKHEGPLHKCDISNSTEA
GQKLFNMLRLGKSEPWTLALENVVGAKNMNVRPLLNYFEPLFTWLKDQNK
NSFVGWSTDWSPYADQSIKVRISLKSALGDKAYEWNDNEMYLFRSSVAYA
MRQYFLKVKNQMILFGEENVRVANLKPRTSFNFTVTAPKNVSDIIPRTEV
EKAIRMSRSRINDAFRLNDNSLEFLGIQPTLGPPNQPPVSIWLIVFGVVM
GVIVVGIVILIFTGIRDRKKKNKARSGENPYASIDISKGENNPGFQNTDD
VQTSF.

In one embodiment, the extracellular domain of the ACE2 protein comprises or consists of the amino acid sequences between a signal sequence and a transmembrane domain of the ACE2 protein but lacks a signal sequence, transmembrane domain and cytosolic domain.

In one embodiment, the extracellular domain of the ACE2 protein consists of or comprises a peptidase domain and collectrin domain. In a further embodiment, the extracellular domain encompasses amino acid residues 18 to 740 of sequence provided in FIG. 4 or SEQ ID NO: 1 (UniProtKB ID: Q9BYF1-1) as shown below or a variant thereof.

(SEQ ID NO: 2)
QSTIEEQAKTFLDKFNHEAEDLFYQSSLASWNYNTNITEENVQ
NMNNAGDKWSAFLKEQSTLAQMYPLQEIQNLTVKLQLQALQQNGSSVLS
EDKSKRINTILNTMSTIYSTGKVCNPDNPQECLLLEPGLNEIMANSLDY
NERLWAWESWRSEVGKQLRPLYEEYVVLKNEMARANHYEDYGDYWRGDY
EVNGVDGYDYSRGQLLEDVERTFEEIKPLYEHLHAYVRAKIMNAYPSYI
SPIGCLPAALLGDMWGREWTNLYSLTVPFGQKPNIDVTDAMVDQAWDAQ
RIFKEAEKFFVSVGLPNMTQGFWENSMLTDPGNVQKAVCHPTAWDLGKG
DFRITACTKVTMDDFLTAHHEMGHIQYDMAYAAQPFLLRNGANEGFREA
VGEIMSLSAATPKHLRSIGLLSPDFQEDNETEINFLLKQALTIVGTLPF
TYMLEKWRWMVFKGEIPKDQWMKKWWERKREIVGVVEPVPHDETYCDPA
SLFHVSNDYSFIRYYTRTLYQFQFQEALCQAAKHEGPLRKCDISNSTEA
GQKLFNMLRLGKSEPWTLALENVVGAKNMNVRPLLNYFEPLFTWLKDQN
KNSFVGWSTDWSPYADQSIKVRISLKSALGDKAYEWNDNEMYLFRSSVA
YAMRQYFLKVKNQMILFGEENVRVANLKPRTSFNFTVTAPKNVSDIIPB
TEVEKAIRMSRSRINDAFRLNDNSLEFLGIQPTLGPPNQPPVS

In another embodiment, the extracellular domain is about 723 amino acids in length.

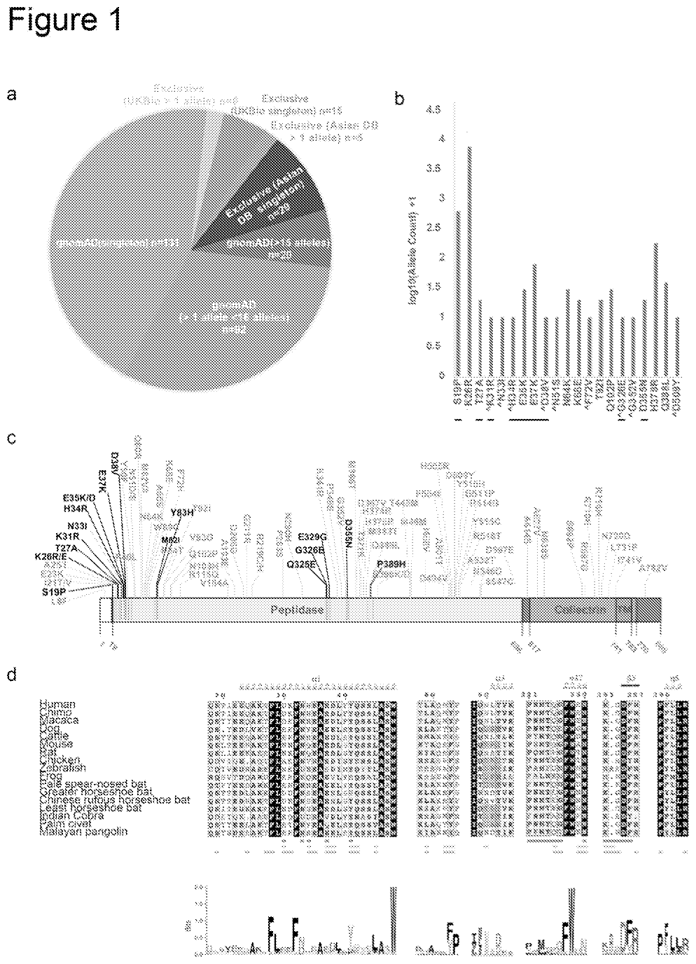

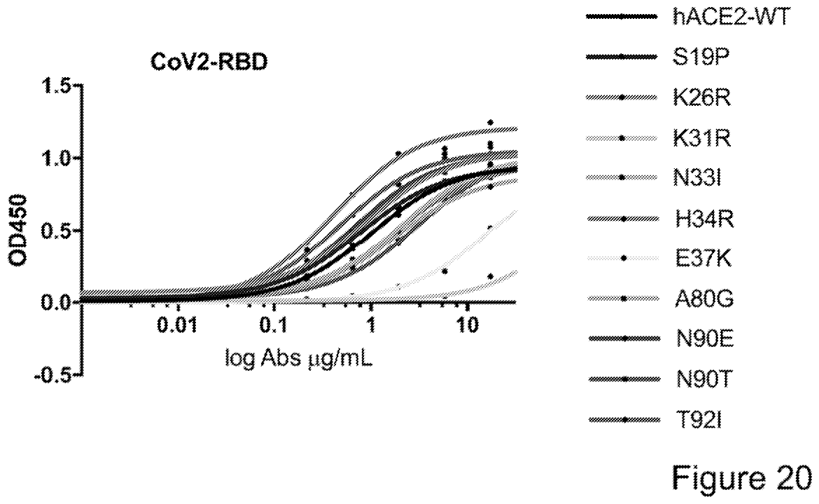

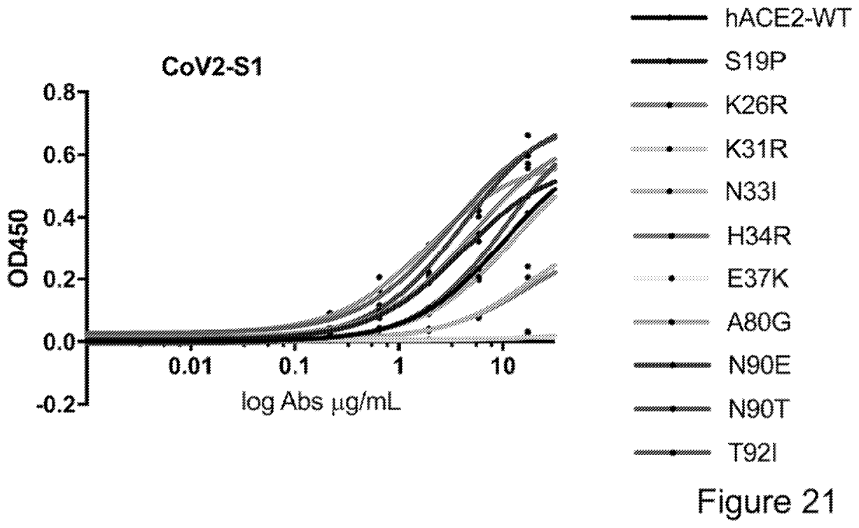

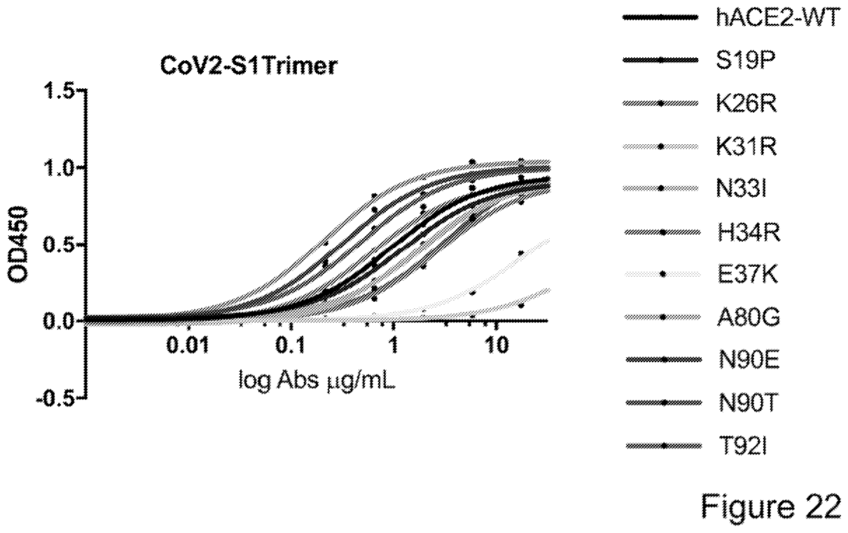

In accordance with the practice of the invention, in one embodiment, the ACE2 variant has at least one amino acid change from a reference full length ACE2 protein as provided in FIG. 4 (SEQ ID NO: 1). The amino acid change may be one or more amino acid substitution. In another embodiment, the amino acid change is a single amino acid substitution. The amino acid change may be an internal deletion or insertion of one or more amino acids. Alternatively, the amino acid change may be an allelic variant change or a combination of allelic variant changes. In one embodiment, the variant is an allelic variant having an amino acid sequence as provided in FIG. 4 and Table 1. In one embodiment, the amino acid change is not an allelic variant change or a combination of allelic variant changes. In another embodiment, the amino acid change is a combination of at least one allelic variant change and at least non-allelic variant change. Examples of the ACE2 variants of the invention include those found in the figures. Examples of ACE2 variants can be found in FIGS. 1, 7, 11, 13, 17-22 and 26. ACE2 variants include allelic variants as well as non-allelic variants. For example, ACE2 non-allelic variants can be synthetic.

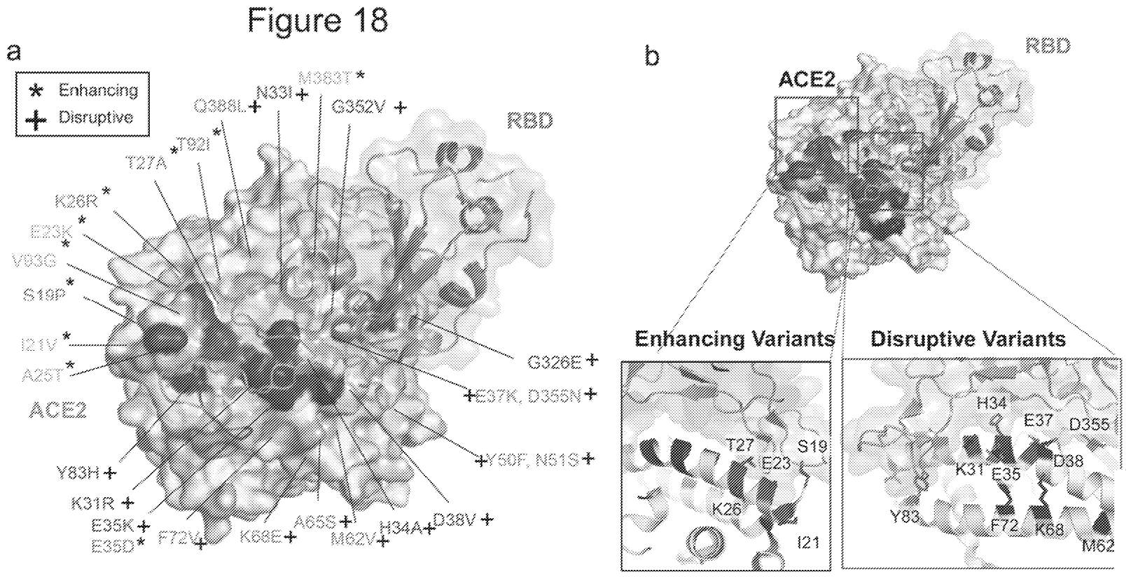

In one embodiment, the amino acid change increases binding or binding affinity of the extracellular domain or fragment thereof for a SARS-CoV-2 virus or a SARS-CoV-2 spike glycoprotein (S-protein) as shown in FIGS. 18, 19-22 and 26 and Table 3. In a further embodiment, the amino acid change is at any of S19, 121, E23, K26, K26, T27, N33, F40, N64, A80, N90, T92, Q102, H378, M383 and T445 and a combination thereof. In another embodiment, the amino acid change is any of S19P, I21V, E23K, K26E, K26R, T27A, N33I, F40L, N64K, A80G, N90I, N90T, T92I, Q102P, H378R, M383T and T445M or a combination thereof.

In one embodiment, the amino acid change prevents glycosylation at amino acid N90. In a further embodiment, the amino acid change which prevents glycosylation at amino acid N90 is substituting asparagine at amino acid residue 90 with another amino acid. In another embodiment, another amino acid is substituted for asparagine. Examples of the amino acid being substituted include, but are not limited to, alanine, arginine, aspartic acid, cysteine, glutamine, glutamic acid, glycine, histidine, isoleucine, leucine, lysine, methionine, phenylalanine, proline, serine, threonine, tryptophan, tyrosine and valine.

In one embodiment, the amino acid change which prevents glycosylation is a change at amino acid residue 91. The leucine at position 91 is substituted with a proline (L91P) or a change at amino acid residue 92, wherein threonine is substituted with another amino acid other than a serine. Examples of the amino acid being substituted for threonine include, but are not limited to, alanine, arginine, asparagine, aspartic acid, cysteine, glutamine, glutamic acid, glycine, histidine, isoleucine, leucine, lysine, methionine, phenylalanine, proline, threonine, tryptophan, tyrosine and valine.

In another embodiment of the isolated SARS-CoV-2 binding protein complex of the invention, the isolated SARS-CoV-2 binding protein complex further comprises a signal sequence located at an amino terminus of the protein.

Examples of the signal sequence include, but are not limited to, SEQ ID NO: 2A-2L as shown below.

(SEQ ID NO: 2A)
MSSSSWLLLSLVAVTAA;
(SEQ ID NO: 2B)
MDWTWRFLFVVAAATGVQS;
(SEQ ID NO: 2C)
MEFGLSWVFLVALFRGVQS;
(SEQ ID NO: 2D)
MELGLSWIFLLAILKGVQC;
(SEQ ID NO: 2E)
MELGLRWVFLVAILEGVQC;
(SEQ ID NO: 2F)
MKHLWFFLLLVAAPRWVLS;
(SEQ ID NO: 2G)
MDWTWRILFLVAAATGAHS;
(SEQ ID NO: 2H)
MEFGLSWLFLVAILKGVQC;
(SEQ ID NO: 2I)
MEFGLSWVFLVALFRGVQC;
(SEQ ID NO: 2J)
MDLLHKNMKHLWFFLLLVAAPRWVLS;
(SEQ ID NO: 2K)
MDMRVPAQLLGLLLLWLSGARC;
and
(SEQ ID NO: 2L)
MKYLLPTAAAGLLLLAAQPAMA.

In one embodiment, the extracellular domain of the ACE2 protein is a variant or allelic variant of amino acid 18-740 of SEQ ID NO: 1 (UniProtKB ID: Q9BYF1-1). In another embodiment, the extracellular domain or fragment thereof comprises a functional peptidase. The functional peptidase may be a carboxypeptidase. The carboxypeptidase may be a metallocarboxypeptidase.

In another embodiment, the extracellular domain or fragment thereof of the ACE2 protein variant comprises a HEXXH zinc-binding motif at amino acids 374 to 378 of FIG. 4 or SEQ ID NO: 1 (UniProtKB ID: Q9BYF1-1). In one embodiment, the HEXXH zinc-binding motif at amino acids 374 to 378 is HEMGH. In a further embodiment, the HEMGH binds a zinc ion, Zn²⁺. In another embodiment, the presence of HEMGH maintains peptidase activity. In yet another embodiment, the peptidase activity is a carboxypeptidase activity.

In another embodiment, the ACE2 extracellular domain or fragment thereof lacks a functional peptidase activity. For example, the functional peptidase activity so lacking may be a carboxypeptidase activity.

In one embodiment, the extracellular domain or fragment thereof of ACE2 protein or ACE2 protein variant comprises an alteration at HEXXH zinc-binding motif corresponding to amino acids 374 to 378 of FIG. 4 or SEQ ID NO: 1 (UniProtKB ID: Q9BYF1-1). In a further embodiment, the alteration in HEXXH zinc-binding motif results in loss of carboxypeptidase catalytic activity and loss of zinc ion binding. In another embodiment, the alteration in HEXXH zinc-binding motif is an amino acid change at histidine 374 and/or histidine 378 in the sequence HEMGH. In one embodiment, the amino acid change is to an amino acid other than a cysteine. In another embodiment, the amino acid change is one or more of alanine, arginine, asparagine, aspartic acid, glutamine, glutamic acid, glycine, isoleucine, leucine, lysine, methionine, phenylalanine, proline, serine, threonine, tryptophan, tyrosine or valine. Examples of the alteration to HEMGH include, but are not limited to, HEMGN, NEMGH, NEMGN, HEMGR, REMGH, NEMGR, REMGN and REMGR. In one embodiment, the alteration to HEMGH is NEMGN. In another embodiment, the alteration to HEMGH is NEMGR.

In another embodiment, the variant comprises an amino acid change at any of S19, E22, E23, Q24, A25, K26, T27, L29, D30, K31, N33, H34, E35, L39, F40, Y41, Q42, A65, W69, F72, E75, Q76, L79, A80, M82, Q89, N90, L91, T92, V93, T324, Q325, N330, L351, G352, D382, A386, P389, R393, S511 and R518 or a combination thereof. Examples of the amino acid change include, but are not limited to, S19V, S19W, S19Y, S19F, S19P, E22T, E23M, E23T, E23Q, E23F, E23C, Q24T, A251, A25V, A25T, A25F, K26I, K26V, K26A, K26D, K26R, T27M, T27L, T27A, T27D, T27K, T27H, 127W, T27Y, T27F, T27C, L29F, D301, D30V, D30E, K31W, K31Y, N33D, N33C, N33I, H34V, H34A, H34S, H34P, E35M, E35V, E35D, E35C, L391, L39V, L39K, L39R, Y41R, Q42M, Q42L, Q42T, Q42V, Q42K, Q42H, Q42C, A65W, W69L, W691, W69V, W69T, W69K, W69C, F72W, F72Y, E75A, E75S, E75T, E75Q, E75K, E75R, E75H, E75W, E75G, Q76M, Q76I, Q76V, Q76T, Q76R, Q76Y, L791, L79V, L79T, L79W, L79Y, L79F, L79P, A80G, M82C, Q89I, Q89D, Q89P, N90M, N90L, N90I, N90V, N90A, N90S, N90T, N90Q, N90D, N90E, N90K, N90R, N90H, N90W, N90Y, N90F, N90P, N900, N90C, L91P, T92M, T92L, T92I, T92V. T92A, T92N, T92Q, T92D, T92E, T92K, T92R, T92H, T92W, T92Y, T92F, T92P, T92G, T92C, V93P, T324A, T324E, T324P, Q325P, N330L, N330H, N330W, N330Y, N330F, L351F, A386L, A386I, P389D, R393K, S511D and R518G or a combination thereof.

In yet another embodiment, the variant comprises an amino acid change at any of S19, E23, A25, K26, T27, D30, K31, N33, H34, L39, Y41, Q42, W69, F72, E75, Q76, L79, A80, Q89, N90, L91, T92, T324, N330, A386 and R393 or a combination thereof. Examples of the amino acid change include, but are not limited to, S19P, E23F, A25V, K26I, K26D, T27M, T27L, T27A, T27D, T27H, T27W, T27Y, T27F, T27C, D30E, K31W, N33D, N33I, H34V, H34A, H34P, L39K, L39R, Y41R, Q42M, Q42L, Q42C, W691, W69V, W69T, W69K, F72Y, E75K, E75R, Q76I, Q76V, Q76T, 1,791, L79V, L79T, L79W, L79Y, L79F, A80G, Q89P, N90M, N90L, N90I, N90V, N90A, N90S, N90T, N90Q, N90D, N90E, N90K, N90R, N90H, N90W, N90Y, N90F, N90P, N90G, N90C, L91P, T92M, T92L, T92I, T92V, T92A, T92N, T92Q, T92D, T92E, T92K, T92R, T92H, T92W, T92Y, T92F, T92P, T92G, T92C, T324E, T324P, N330L, N330H, N330W, N330Y, N330F, A386L and R393K or a combination thereof.

In another embodiment, the variant comprises an amino acid change at any of S19, E22, E23, Q24, A25, K26. T27, L29, D30, K31, N33, H34, E35, L39, Q42, A65, W69, F72, F75, Q76, L79, A80, M82, Q89, T92, V93, T324, Q325, L351, A386, P389, S511 and R518 or a combination thereof. Examples of the amino acid change include, but are not limited to, S19V, S19W, S19Y, S19F, E22T, E23M, E23T, E23Q, E23C, Q24T, A251, A25T, A25F, K26V, K26A, K26R, T27K, L29F, D301, D30V, K31Y, N33C, N33I, H34S, E35M, E35V, E35D, E35C, L391, L39V, Q421, Q42V, Q42K, Q42H, A65W, W69L, W69C, F72W, E75A, E75S, E75T, E75Q, E75H, E75W, E75G, Q76M, Q76R, Q76Y, L79P, A80G, M82C, Q89I, Q89D, T92I, V93P, T324A, Q325P, L351F, A386I, P389D, S511D and R518G or a combination thereof.

In another embodiment, the variant comprises an amino acid change at any of S19, I21, E23, K26, T27, N33, F40, Q60, N64, A80, N90, T92, Q102, H378, M383, T445 and Y510 or a combination thereof. Examples of the amino acid change include, but are not limited to, S19P, I21V, E23K, K26E, K26R, T27A, F40L, Q60R, N64K, N90I, N90T, T92I, Q102P, H378R, M383T, T445M and Y510H or a combination thereof.

In yet another embodiment, the allelic variant comprises an amino acid change at any of 519, 121, E23, K26, T27, N33, F40, Q60, N64, A80, T92, Q102, H378, M383, T445 and Y510 or a combination thereof. Examples of the amino acid change include, but are not limited to, S19P, 21V, E23K, K26E, K26R, T27A, N33I, F40L, Q60R, N64K, A80G, T92I, Q102P, H378R, M383T, T445M and Y510H or a combination thereof.

In another embodiment, the allelic variant comprises an amino acid change at any of S19, T27, N33I, A80G and T92 or a combination thereof. Examples of the amino acid change include, but are not limited to, S19P, T27A, N33I, A80G and T92I and a combination thereof.

In another embodiment, the allelic variant comprises an amino acid change at any of I21, K26, N64, Q102 and H378 or a combination thereof. Examples of the amino acid change include, but are not limited to, I21V, K26R, N64K, Q102P and H378R or a combination thereof.

In another embodiment, the variant comprises an amino acid change at any of E23, K26, F40, Q60, M383, T445 and Y510 or a combination thereof. Examples of the amino acid change include, but are not limited to, E23K, K26E, F40L, Q60R, M383T, T445M and Y510H or a combination thereof.

In yet another embodiment, the variant comprises an amino acid change at any of S19, 121, E23, K26, T27, F40, N64, N90, T92, Q102, H378, M383 and T445 or a combination thereof. Examples of the amino acid change include, but are not limited to, S19P, I21V, E23K, K26E, K26R, T27A, F40L, N64K, N90I, N90T, T92I, Q102P, H378R, M383T and T445M or a combination thereof.

In another embodiment, the variant comprises amino acid changes at amino acid S19, 121, E23, K26, T27, F40, N64, N90, T92, Q102, H378, M383 and T445. In a further embodiment, the variant comprises amino acid changes S19P, 21V, E23K, K26E, T27A, F40L, N64K, N90I, N90T, T92I, Q102P, H378R, M383T and T445M. In another embodiment, the variant comprises amino acid changes S19P, I21V, E23K, K26R, T27A, F40L, N64K, N90L N90T, T92I, Q102P, H378R, M383T and T445M.

In one embodiment, the fragment of ACE2 extracellular domain consists of peptidase or carboxypeptidase domain.

In another embodiment, the fragment of ACE2 extracellular domain lacks a signal peptide or sequence, collectrin domain, transmembrane domain and cytosolic domain. In a further embodiment, the peptidase or carboxypeptidase domain consists of or comprises amino acid residues 18-615 as provided in FIG. 4 or SEQ ID NO: 1 (UniProtKB ID: Q9BYF1-1) and as shown below:

(SEQ ID NO: 3)
QSTIEEQAKTFLDKFNHEAEDLFYQSSLASWNYNTNITEENVQ
NMNNAGDKWSAFLKEQSTLAQMYPLQEIQNLTVKLQLQALQQNGSSVLSE
DKSKRLNTILNTMSTIYSTGKVCNPDNPQECLLLEPGLNEIMANSLDYNE
RLWAWESWRSEVGKQLRPLYEEYVVLKNEMARANHYEDYGDYWRGDYEVN
GVDGYDYSRGQLIEDVEHTFEEIKPLYEALHAYVRAKLMNAYPSYISPIG
CLPAHLLGDMWGRFWTNLYSLTVPFGQKPNIDVTDAMVDQAWDAQRIFKE
AEKFFVSVGLPNMTQGFWENSMLTDPGNVQKAVCHPTAWDLGKGDFRILM
CTKVTMDDFLTAHHEMGHIQYDMAYAAQPFLLRNGANEGFHEAVGEIMSL
SAATPKHLKSIGLLSPDFQEDNETEINFLLKQALTIVGTLPFTYMLEKWR
WMVFKGEIPKDQWMKKWWEMKREIVGVVEPVPHDETYCDPASLFHVSNDY
SFIRYYTRTLYQFQFQEALCQAAKHEGPLHKCDISNSTEAGQKLFNMLRL
GKSEPWTLALENVVGAKNMNVRPLLNYFEPLFTWLKDQNKNSFVGWSTDW
SPYAD

or a variant thereof.

In one embodiment, the fragment of ACE2 extracellular domain consists of or comprises about 598 amino acids. In another embodiment, the fragment of ACE2 extracellular domain is greater than about 5 amino acids. In another embodiment, the fragment of ACE2 extracellular domain is less than about 723 amino acids. In yet another embodiment, the fragment of ACE2 extracellular domain consists or comprises between about 10 and 723 amino acids. In another embodiment, the fragment of ACE2 extracellular domain consists or comprises between about 601 and 700 amino acids. In another embodiment, the fragment of ACE2 extracellular domain consists or comprises between about 501 and 600 amino acids. In another embodiment, the fragment of ACE2 extracellular domain consists or comprises between about 401 and 500 amino acids. In another embodiment, the fragment of ACE2 extracellular domain consists or comprises between about 301 and 400 amino acids. In another embodiment, the fragment of ACE2 extracellular domain consists or comprises between about 201 and 300 amino acids. In another embodiment, the fragment of ACE2 extracellular domain consists or comprises between about 101 and 200 amino acids. In another embodiment, the fragment of ACE2 extracellular domain consists or comprises between about 50 and 100 amino acids. In another embodiment, the fragment of ACE2 extracellular domain consists or comprises between about 25 and 65 amino acids. In another embodiment, the fragment of ACE2 extracellular domain consists or comprises between about 9 and 35 amino acids.

In one embodiment, the extracellular domain fragment consists of amino acid residues 18-393 as provided in FIG. 4 or SEQ ID NO: 1 (UniProtKB ID: Q9BYF1-1) and as shown below:

(SEQ ID NO: 4)
QSTIEEQAKTFLDKFNHEAEDLFYQSSLASWNYNTNITEENVQ
NMNNAGDKWSAFLKEQSTLAQMYPLQEIQNLTVKLQLQALQQNGSSVLSE
DKSKRLNTILNTMSTIYSTGKVCNPDNPQECLLLEPGLNEIMANSLDYNE
RLWAWESWRSEVGKQLRPLYEEYVVLKNEMARANHYEDYGDYWRGDYEVN
GVDGYDYSRGQLIEDVEHTFEEIKPLYEALHAYVRAKLMNAYPSYISPIG
CLPAHLLGDMWGRFWTNLYSLTVPFGQKPNIDVTDAMVDQAWDAQRIFKE
AEKFFVSVGLPNMTQGFWENSMLTDPGNVQKAVCHPTAWDLGKGDFRILN
CTKVTMDDFLTAHHEMGHIQYDMAYAAQPFLLR

or a variant thereof or a portion thereof, wherein the portion is 35 or more amino acids.

In one embodiment, the fragment of ACE2 extracellular domain consists of or comprises about 376 amino acids.

In another embodiment, the extracellular domain fragment consists of or comprises amino terminus of ACE2 extracellular domain. In another embodiment, the amino terminus of ACE2 extracellular domain consists or comprises amino acid residues 18-48 as provided in FIG. 4 or SEQ ID NO: 1 (UniProtKB ID: Q9BYF1-1) and as shown below:

(SEQ ID NO: 5)
QSTIEEQAKTFLDKFNHEAEDLFYQSSLASW

or a variant thereof.

In another embodiment, the fragment of ACE2 extracellular domain consists of or comprises about 31 amino acids.

In one embodiment of the isolated SARS-CoV-2 binding protein complex of the invention, the complex further comprises at least one additional extracellular domain fragment such that two or more extracellular domain fragments are functionally linked so as to permit binding to SARS-CoV-2 virus or SARS-CoV-2 spike glycoprotein (S-protein), wherein each extracellular domain fragment consists of or comprises a polypeptide secondary structural element. In a further embodiment, a polypeptide secondary structural element is any of helix, alpha helix, 3₁₀ helix, π helix, β-turn, hydrogen bonded turn, extended strand in parallel and/or antiparallel β-sheet conformation, residue in isolated β-bridge, bend and coil.

Examples of the extracellular domain fragment include, but are not limited to, a helix forming peptide, TEENVQNMNNAGDKWSAFLKEQSTLAQMY (SEQ ID NO: 6), corresponding to amino acid residue 55-83 as provided in FIG. 4 or SEQ ID NO: 1 (UniProtKB ID: Q9BYF1-1) or a variant thereof or a fragment thereof; a helix forming peptide, EEQAKTFLDKFNIIEAEDLFYQSSLASWNYNT (SEQ ID NO: 7), corresponding to amino acid residue 22-52 as provided in FIG. 4 or SEQ ID NO: 1 (UniProtKB ID: Q9BYF1-1) or a variant thereof or a fragment thereof; and, a β-turn peptide, AWDLGKGDFR (SEQ ID NO: 8), corresponding to amino acid residue 348-357 as provided in FIG. 4 or SEQ ID NO: 1 (UniProtKB ID: Q9BYF1-1) or a variant thereof or a fragment thereof.

In one embodiment, the fragment of a helix forming peptide of SEQ ID NO: 6 is: AGDKWSAFLKEQSTLAQMY (SEQ ID NO: 9), corresponding to amino acid residue 65-83 as provided in FIG. 4 or SEQ ID NO: 1 (UniProtKB ID: Q9BYF1-1) or a variant thereof.

In another embodiment, the fragment of a helix forming peptide of SEQ ID NO: 7 is: EEQAKTFLDKFNHEAEDLFYQSS (SEQ ID NO: 10), corresponding to amino acid residue 22-44 as provided in FIG. 4 or SEQ ID NO: 1 (UniProtKB ID: Q9BYF1-1) or a variant thereof.

In another embodiment, the fragment of a β-turn peptide of SEQ ID NO: 8 is: DLGKGDFR (SEQ ID NO: 11), corresponding to amino acid residue 350-357 as provided in FIG. 4 or SEQ ID NO: 1 (UniProtKB ID: Q9BYF1-1) or a variant thereof.

In another embodiment, two or more extracellular domain fragments are ordered and covalently linked to form a polypeptide chain. In another embodiment, the extracellular domain fragments are in the same order or form overlapping fragments having an order as present in the primary amino acid sequence of ACE2 protein. In a further embodiment, the order from amino-to-carboxyl direction is: [helix forming peptide with SEQ ID NO: 7] or [helix forming peptide with SEQ ID NO: 10] followed by [helix forming peptide with SEQ ID NO: 6] or [helix forming peptide with SEQ ID NO: 9] and lastly followed by [β-turn peptide of SEQ ID NO: 8] or [β-turn peptide of SEQ ID NO: 11].

In another embodiment, the order from amino-to-carboxyl direction is: [helix forming peptide with SEQ ID NO: 7] or [helix forming peptide with SEQ ID NO: 10] followed by [helix forming peptide with SEQ ID NO: 6] or [helix forming peptide with SEQ ID NO: 9].

In another embodiment, the order from amino-to-carboxyl direction is: [helix forming peptide with SEQ ID NO: 6] or [helix forming peptide with SEQ ID NO: 9] and lastly followed by [β-turn peptide of SEQ ID NO: 8] or [β-turn peptide of SEQ ID NO: 11].

In yet another embodiment, the order from amino-to-carboxyl direction is: [helix forming peptide with SEQ ID NO: 7] or [helix forming peptide with SEQ ID NO: 10] followed by [β-turn peptide of SEQ ID NO: 8] or [β-turn peptide of SEQ ID NO: 11].

In another embodiment, the order from amino-to-carboxyl direction is: [helix forming peptide with SEQ ID NO: 7] followed by [helix forming peptide with SEQ ID NO: 6] and lastly followed by [β-turn peptide of SEQ ID NO: 8].

In an additional embodiment, the order from amino-to-carboxyl direction is: [helix forming peptide with SEQ ID NO: 10] followed by [helix forming peptide with SEQ ID NO: 9] and lastly followed by [0-turn peptide of SEQ ID NO: 11].

In yet another embodiment, the order from amino-to-carboxyl direction is: [helix forming peptide with SEQ ID NO: 7] followed by [β-turn peptide of SEQ ID NO: 11].

Further still, in one embodiment, the order from amino-to-carboxyl direction is: [helix forming peptide with SEQ ID NO: 10] followed by [β-turn peptide of SEQ ID NO: 8].

In one embodiment, the extracellular domain fragments are ordered such that at least one fragment is not in the same order as present in the primary amino acid sequence of ACE2 protein. In a further embodiment, the at least one fragment that is not in the same order has the following from amino-to-carboxyl direction: [helix forming peptide with SEQ ID NO: 6] or [helix forming peptide with SEQ ID NO: 9] followed by [helix forming peptide with SEQ ID NO: 7] or: [helix forming peptide with SEQ ID NO: 10] and lastly by [β-turn peptide of SEQ ID NO: 8] or [β-turn peptide of SEQ ID NO: 11]. In another embodiment, the at least one fragment that is not in the same order has the following from amino-to-carboxyl direction: [helix forming peptide with SEQ ID NO: 6]-[helix forming peptide with SEQ ID NO: 7]-[β-turn peptide of SEQ ID NO: 8]. In yet another embodiment, the at least one fragment that is not in the same order has the following from amino-to-carboxyl direction: [helix forming peptide with SEQ ID NO: 9]-[helix forming peptide with SEQ ID NO: 10]-[β-turn peptide of SEQ ID NO: 11].

In one embodiment of the invention, the fragments are separated by a peptide linker. The peptide linker may be between one to ten amino acids. The peptide linker may be glycine and/or serine rich. Examples of the peptide linker include, but are not limited to, G, GG, and GGGGSGG.

In one embodiment of the invention, the variant may be variant, allelic variant or combination of variants and/or allelic variants. In a further embodiment, the variant, allelic variant or combination of variants and/or allelic variants comprise one or more amino acid substitution relative to reference ACE2 protein sequence (FIG. 4) and occur at amino acid residue and substitution as described in figures or mutations described herein. In another embodiment, the one or more amino acid substitution increases binding or binding affinity of ACE2 variant fragment for SARS-CoV-2 virus or SARS-CoV-2 S-protein.

In one embodiment of the invention, the antibody is an immunoglobulin. The immunoglobulin may comprise an immunoglobulin heavy chain. The immunoglobulin may comprise an immunoglobulin light chain. The immunoglobulin may comprise an immunoglobulin heavy chain and an immunoglobulin light chain. Examples of the immunoglobulin include, but are not limited to, IgM, IgG, IgA, IgD and IgE. In a preferred embodiment of the invention, the immunoglobulin is IgG. Examples of the IgG include, but are not limited to, IgG1, IgG2, IgG3 and IgG4.

In one embodiment, the immunoglobulin binds an antigen on SARS-CoV-2 virus or SARS-CoV-2 spike glycoprotein (S-protein). In another embodiment, the immunoglobulin is derived from a hybridoma. In yet another embodiment, the immunoglobulin is produced by recombinant DNA method or molecular biology method. In another embodiment, the immunoglobulin is derived from a Fab library. In another embodiment, the immunoglobulin is derived from a single chain variable antibody fragment (scFv) phage display library.

In one embodiment, the Fab library or scFv phage display library comprises a binding protein for SARS-CoV-2 virus or SARS-CoV-2 protein, wherein the binding protein does not compete with ACE2 binding of SARS-CoV-2 virus or SARS-CoV-2 protein. In a further embodiment, the binding protein is CR3022 scFv which binds SARS-CoV-2 virus and SARS-CoV-2 S-protein (ter Meulen J, van den Brink E N, Poon L L M, Marissen W E, Leung C S W, et al. (2006) Human monoclonal antibody combination against SARS coronavirus: Synergy and coverage of escape mutants. PLoS Med 3(7): e237. DOI: 10.1371/journal.pmed.0030237).

In one embodiment, the immunoglobulin is obtained after converting CR3022 scFv to an immunoglobulin format. In another embodiment, the immunoglobulin is a recombinant protein. In another embodiment, the immunoglobulin is from a mammal or classified as being from a mammal. Examples of the mammal include, but are not limited to, mouse, rat, dog, cat, civet, pangolin, bat, pig, guinea pig, goat, sheep, donkey, horse, camel, chimpanzee, monkey, gorilla, cattle, and human. In a preferred embodiment, the mammal is human. In another embodiment, the immunoglobulin is from a chicken or classified as being from a chicken. In another embodiment, the immunoglobulin is a full-length immunoglobulin. In another embodiment, the full-length immunoglobulin is derived from converting a Fab or scFv to a full-length immunoglobulin. In a further embodiment, the Fab or scFv binds SARS-CoV-2 virus or SARS-CoV-2 S-protein but does not compete with ACE2 binding to SARS-CoV-2 virus or SARS-CoV-2 protein. In another embodiment, the scFv that binds SARS-CoV-2 virus or SARS-CoV-2 S-protein is CR3022 scFv. In yet another embodiment, the scFv that binds SARS-CoV-2 virus or SARS-CoV-2 S-protein is a variant of CR3022 scFv, wherein one or more amino acid change in complement-determining regions (CDRs) increases binding affinity of the variant to SARS-CoV-2 virus or SARS-CoV-2 S-protein without competing with ACE2 binding to SARS-CoV-2 virus or SARS-CoV-2 protein.

3) In one embodiment of the invention, the antibody fragment is a fragment or portion of an immunoglobulin. Examples of the fragment or portion of an immunoglobulin include, but are not limited to, Fab, Fab′, F(ab′)₂, F_{c}, single chain variable fragment (scFv), diabody and recombinantly produced immunoglobulin fragment and a combination thereof. In another embodiment, the antibody fragment is a scFv, which does not compete with ACE2 binding to SARS-CoV-2 virus or SARS-CoV-2 protein. Further, in another embodiment, the scFv is CR3022 scFv.

In one embodiment, the scFv is a variant of CR3022 scFv, wherein one or more amino acid change in CDRs increases binding affinity of the variant to SARS-CoV-2 virus or SARS-CoV-2 S-protein without competing with ACE2 binding to SARS-CoV-2 virus or SARS-CoV-2 protein. In another embodiment, the antibody fragment is not a scFv but is derived from a scFv and wherein the antibody fragment does not compete with ACE2 binding to SARS-CoV-2 virus or SARS-CoV-2 protein. In a further embodiment, the scFv is CR3022 scFv. In another embodiment wherein the scFv is a variant of CR3022 scFv, one or more amino acid change in CDRs increases binding affinity of the variant to SARS-CoV-2 virus or SARS-CoV-2 S-protein without competing with ACE2 binding to SARS-CoV-2 virus or SARS-CoV-2 protein.

In one embodiment of the invention, the antibody fragment is a Fab. In another embodiment, the antibody fragment is a Fab′. In yet another embodiment, the antibody fragment is a F(ab′)2. In another embodiment, the antibody fragment is a diabody or a scFv.

In one embodiment of the invention, the antibody fragment binds SARS-CoV-2 virus or SARS-CoV-2 S-protein without competing with ACE2 binding to SARS-CoV-2 virus or SARS-CoV-2 protein. In another embodiment, the antibody fragment is derived from CR3022 scFv. In another embodiment, the antibody fragment is derived from a variant of CR3022 scFv, wherein one or more amino acid change in CDRs increases binding affinity of the variant to SARS-CoV-2 virus or SARS-CoV-2 S-protein without competing with ACE2 binding to SARS-CoV-2 virus or SARS-CoV-2 protein. In another embodiment, the antibody fragment is a Fe. In another embodiment, the antibody fragment is recombinantly produced immunoglobulin fragment obtained by recombinant DNA method or molecular biology method.

In one embodiment of the invention, the antibody or antibody fragment comprises a Fc with functional Fc effector functions. In another embodiment, the antibody or antibody fragment comprises a Fc mutated so as to reduce or abolish Fc effector function. In another embodiment, the Fc effector function is to support binding of Fc receptor and/or complement protein 1q (C1q). In another embodiment, the Fc effector function is antibody-dependent cellular cytotoxicity (ADCC), antibody-dependent cellular phagocytosis (ADCP), complement-dependent cytotoxicity (CDC) or a combination thereof. In another embodiment, the mutated Fc has one or more amino acid change. In a further embodiment, the amino acid change decreases or abolishes binding of the Fc receptor or complement protein 1q (C1q) to the antibody or antibody fragment. In another embodiment, the amino acid change decreases or abolishes binding of the Fcγ receptor or complement protein 1q (C1q) to IgG or IgG fragment. In another embodiment, the Fcγ receptor is any of Fcγ receptor 1, Fcγ receptor II and Fcγ receptor III and a combination thereof.

In one embodiment, the amino acid change decreases or abolishes antibody-dependent cellular cytotoxicity (ADCC), antibody-dependent cellular phagocytosis (ADCP), complement-dependent cytotoxicity (CDC) or a combination thereof. In another embodiment, the amino acid change is at aspartic acid 265, asparagine 297 or both for IgG or equivalent, wherein equivalent is one or more amino acid change at other amino acid position of IgG reducing or abolishing Fe effector function or at a corresponding position or other position for IgM, IgD, IgA or IgE. In another embodiment, the amino acid change is D265A or N297G or both. In yet another embodiment, the amino acid change is D265A and N297G.

In one embodiment, the combination comprises two or more antibody fragments. In another embodiment, the combination comprises a Fc and a diabody or scFv. In a further embodiment, the Fc and the diabody or scFv are covalently linked. In another embodiment, the Fc and the diabody or scFv are covalently linked through a linker. In yet another embodiment, the linker is a peptide linker. In another embodiment, the Fc is linked to the amino terminus of the diabody or scFv.

In one embodiment of the invention, the isolated SARS-CoV-2 binding protein complex is a bi-specific protein. In another embodiment, the bispecific protein binds two different determinants on SARS-CoV-2 virus or SARS-CoV-2 S-protein. In another embodiment, one specificity is conferred by an antigen-binding determinant of an immunoglobulin component and other specificity is conferred by an ACE2 component, wherein antigen binding site and ACE2 binding site of SARS-CoV-2 virus or SARS-CoV-2 S-protein do not overlap and both sites can be occupied at the same time by the antigen-binding determinant of an immunoglobulin and ACE2.

In one embodiment of the invention, the antigen-binding determinant of an immunoglobulin component consists of or comprises a light chain and a heavy chain of an immunoglobulin. In another embodiment, the light chain consists of or comprises a variable domain, V_{L}, and a constant domain, C_{L}. In another embodiment, the heavy chain consists of or comprises a variable domain, V_{H}, and three constant domains, C_{H}1, C_{H}2 and C_{H}3. In another embodiment, the heavy chain further comprises a hinge region. In another embodiment, the heavy chain further comprises am additional constant domain, C_{H}4.

In one embodiment of the invention, the antigen-binding determinant of an immunoglobulin does not compete with ACE2 binding at SARS-CoV-2 virus or SARS-CoV-2 S-protein. In another embodiment, the antigen-binding determinant is that of CR3022 scFv or comprises CDRs of CR3022 scFv. In yet another embodiment, the CDRs of CR3022 scFv are defined by Kabat method or IMGT method.

In one embodiment, the antibody, antibody fragment, immunoglobulin, diabody, scFv or Fc is human or humanized. In another embodiment, the ACE2 component consists of or comprises ACE2 extracellular domain, its variant or fragment thereof and an immunoglobulin heavy chain of a F_{c} fragment. In another embodiment, the ACE2 extracellular domain, its variant or fragment thereof is linked at its C-terminus to the immunoglobulin heavy chain of a Fe fragment. In another embodiment, the ACE2 extracellular domain or fragment thereof has a sequence as described in any of the figures or SEQ ID NO: 2-11. In a preferred embodiment, the ACE2 extracellular domain fragment is SEQ ID NO: 3. In another embodiment, the variant may be a variant, allelic variant or combination of variants and/or allelic variants. In another embodiment, the variant, allelic variant or combination of variants and/or allelic variants comprise one or more amino acid substitutions relative to reference ACE2 protein sequence (FIG. 4) and occur at amino acid residue and substitution as described in FIG. 1c or an amino acid substitution of reference an ACE2 (or variant or fragment thereof) of FIG. 4 may be at any of S19,121, E23, K26, K26, T27, N33, F40, N64, A80, N90, T92, Q102, H378, M383 and T445, S19P, I21V, E23K, K26E, K26R, T27A, N33I, F40L, N64K, A80G, N90I, N90T, T92I, Q102P, H378R, M383T and T445M or a combination thereof. In a specific embodiment, the amino acid change may prevent glycosylation at amino acid N90 of reference ACE2 of FIG. 4. For example, the amino acid change which may prevent glycosylation at amino acid N90 may be a change which involves substituting asparagine at amino acid residue 90 with another amino acid which may include, any of alanine, arginine, aspartic acid, cysteine, glutamine, glutamic acid, glycine, histidine, isoleucine, leucine, lysine, methionine, phenylalanine, proline, serine, threonine, tryptophan, tyrosine and valine.

In one embodiment, these amino acid substitutions may comprise an alteration at an HEXXH zinc-binding motif corresponding to amino acids 374 to 378 of FIG. 4 or SEQ ID NO: 1 (UniProtKB ID: Q9BYF1-1). In accordance with the practice of the invention, the alteration in the HEXXH zinc-binding motif may result in a loss of carboxypeptidase catalytic activity and/or a loss of zinc ion binding. For example, the alteration in the HEXXH zinc-binding motif may be an amino acid change at histidine 374 and/or histidine 378 in the sequence HEMGH. The amino acid change may be to an amino acid other than a cysteine. For example, histidine 374 and/or histidine 378 in the sequence HEMGH may be changed to any of an alanine, arginine, asparagine, aspartic acid, glutamine, glutamic acid, glycine, isoleucine, leucine, lysine, methionine, phenylalanine, proline, serine, threonine, tryptophan, tyrosine or valine. In specific examples, the HEMGH so altered may become any of HEMGN, NEMGH, NEMGN, HEMGR, REMGH, NEMGR, REMGN and REMGR. In one embodiment, the alteration to HEMGH results in NEMGN. In another embodiment, HEMGH is altered to become NEMGR.

The amino acid substitutions at reference ACE2 protein may be at any of S19, 121, E22, E23, Q24, A25, K26, T27, L29, D30, K31, N33, H34, E35, L39, F40, Y41, Q42, Q60, N64, A65, W69, F72, E75, Q76, L79, A80, M82, Q89, N90, L91, T92, V93, Q102, T324, Q325, N330, L351, H378, M383, A386, P389, R393, T445, Y510, S511, R518, S19P, S19V, S19W, S19Y, S19F, I21V, E22T, E23F, E23K, E23M, E23T, E23Q, E23C, Q24T, A25L, A25T, A25F, A25V, K26V, K26A, K26D, K26E, K26R, K26I, K26R, K31 W, T27K, T27M, T27L, 127A. T27D, T27H, 127W, T27Y, T27F, T27C, L29F, D30E, D301, D30V, K31Y, N33C, N33D, N33I, H34S, 1134V, H34A, 134P, E35C, E35D, E35M, E35V, L391, L39V, L39K, L39R, F40L, Y41R, Q42V, Q42K, Q42H, Q42M, Q42L, Q42C, Q421, Q60R, N64K, A65W, W69L, W69C, W691, W69V, W69T, W69K, F72W, F72Y, E75A, E75K, E75R, E75S, E75T, E75Q, E75H, E75W, E75G, Q76M, Q76R, Q76Y, Q76I, Q76V, Q76T, L791, L79P, L79V, L79T, L79W, L79Y, L79F, A80G, M82C, Q89L, Q89D, Q89P, N90M, N90L, N90I, N90V, N90A, N90S, N90T, N90Q, N90D, N90E, N90K, N90R, N90H, N90W, N90Y, N90F, N90P, N90G, N90C, L91P, T92M, T92L, T92I, T92V, T92A, T92N, T92Q, T92D, T92E, T92K, T92R, T92H, T92W, T92Y, T92F, T92P, T92G, T92C, V93P, Q102P, T324A, T324E, T324P, Q325P. N330L, N330H, N330W, N330Y, N330F, L351F, H378R, M383T, A386I, A386L, P389D, R393K, T445M, Y510H, S511D and R518G, or a combination thereof.

In a specific embodiment, the allelic variant of a reference ACE2 protein comprises an amino acid change at any of S19, T27, N33, A80 and T92 or a combination thereof. For example, the amino acid change may include, but are not limited to any of S19P, T27A, A33, A80G and T92I and a combination thereof. In another embodiment, the allelic variant comprises an amino acid change at any of I21, K26, N64, Q102 and H378 or a combination thereof. For example, the amino acid change may include, but are not limited to any of I21V, K26R, N64K, Q102P and H378R or a combination thereof. In yet another embodiment, the variant comprises an amino acid change at any of E23, K26, F40, Q60, M383, T445 and Y510 or a combination thereof. For example, the amino acid change may include, but are not limited to any of E23K, K26E, F40L, Q60R, M383T, T445M and Y510H or a combination thereof.

In a specific embodiment, the variant of a reference ACE2 protein comprises an amino acid change at any of S19, 121, E23, K26, T27, N33, F40, N64, A80, N90, T92, Q102, H378, M383 and T445 or a combination thereof. For example, the amino acid change may include, but are not limited to, any of S19P, I21V, E23K, K26E, K26R, T27A, N33I, F40L, N64K, A80G, N90I, N90T, T92I, Q102P, H378R, M383T and T445M or a combination thereof. In another embodiment, the variant comprises amino acid changes at amino acid S19, 121, E23, K26, 127, N33, F40, N64, A80, N90, T92, Q102, 1378, M383 and T445. For example, the variant may comprise amino acid changes S19P, I21V, E23K, K26E, T27A, N33I, F40L, N64K, A80G, N90I, N90T, T92I, Q102P, H378R, M383T and T445M or, the variant may comprise amino acid changes S19P, I21V, E23K, K26R, T27A, F40L, N64K, N90I, N90T, T92I, Q102P, H378R, M383T and T445M.

In another embodiment, the ACE2 variant lacks peptidase or carboxypeptidase activity. In another embodiment, the variant comprises H374N, H378N or both. In another embodiment, the variant comprises H374N and H378N.

In one embodiment, the ACE2 extracellular domain, its variant or fragment thereof is directly linked to the immunoglobulin heavy chain of a Fe fragment without a linker to produce a single polypeptide chain. In another embodiment, a linker is used link to the immunoglobulin heavy chain. In a further embodiment, the linker is a peptide linker. In another embodiment, the peptide linker is between one to twenty amino acids. In another embodiment, the peptide linker is glycine and/or serine rich. Examples of the peptide linker include, but are not limited to G, GG, and GGGGSGG.

In one embodiment, the immunoglobulin heavy chain of a F_{c} fragment comprises C_{H}2 and C_{H}3 constant domains. In another embodiment, the immunoglobulin heavy chain of a F_{c} fragment further comprises a hinge region. In another embodiment, the immunoglobulin heavy chain of a Fₑ fragment further comprises C_{H}4 constant domain. In another embodiment, the immunoglobulin heavy chain of a F_{c} fragment comprises C_{H}2 and C_{H}3 constant domains and a hinge region.

In one embodiment, the bispecific protein consists or comprises an immunoglobulin heavy chain comprising a variable domain, V_{H}, three constant domains, C_{H}1, C_{H}2 and C_{H}3, and a hinge region and an immunoglobulin light chain comprising a variable domain, V_{L}, and a constant domain, C_{L}, to form an antigen-binding determinant which binds to SARS-CoV-2 virus or SARS-CoV-2 S-protein but does not compete with ACE2 binding; and a third polypeptide comprising an ACE2 extracellular domain fragment comprising one or more amino acid change reducing or abolishing peptidase or carboxypeptidase activity linked to an immunoglobulin heavy chain, constant region fragment, an Fc fragment, comprising a hinge region and C_{H}2 and C_{H}3 constant domains.

Examples of the ACE2 extracellular domain fragment include, but are not limited to, a polypeptide from amino acid residue 1-740 of SEQ ID NO: 1, a polypeptide from amino acid residue 1-615 of SEQ ID NO: 1, a polypeptide from amino acid residue 1-393 of SEQ ID NO: 1, a polypeptide with SEQ ID NO: 2, a polypeptide with SEQ ID NO: 3, and a polypeptide with SEQ ID NO: 4 and variant thereof and wherein the polypeptide comprises one or more amino acid change that reduces or abolishes peptidase or carboxypeptidase activity.

In one embodiment, the ACE2 extracellular domain fragment is a polypeptide from amino acid residue 1-615 of SEQ ID NO: 1, a polypeptide with SEQ ID NO: 2 or variant thereof and wherein the polypeptide comprises one or more amino acid change that reduces or abolishes peptidase or carboxypeptidase activity.

In another embodiment, the ACE2 extracellular domain fragment comprises a polypeptide with SEQ ID NO: 2 or variant thereof and wherein the polypeptide comprises one or more amino acid change that reduces or abolishes peptidase or carboxypeptidase activity. In another embodiment, the ACE2 extracellular domain fragment is a polypeptide from amino acid residue 1-615 of SEQ ID NO: 1 and wherein the polypeptide comprises one or more amino acid change that reduces or abolishes peptidase or carboxypeptidase activity. Examples of the one or more amino acid change that reduces or abolishes peptidase or carboxypeptidase activity may include, but are not limited to, H374N, H378N, H378R, both H1374N and H378N, and both H374N and H378R.

In one embodiment, the variant comprises one or more amino acid substitution which increases binding or binding affinity of the ACE2 fragment for SARS-CoV-2 virus or SARS-CoV-2 S-protein. In another embodiment, the immunoglobulin heavy chain constant domains additionally comprise one or more amino acid changes based on a “knob-in-hole” protein design principle, wherein the changes favor heterodimer formation between the immunoglobulin heavy chain comprising a heavy chain variable domain and the fragment of an immunoglobulin heavy chain linked to ACE2. In a further embodiment, the amino acid changes are in C_{H}3 constant domain. In a further embodiment, the C_{H}3 constant domain of a first heavy chain comprises at least one amino acid change to introduce a “knob” or “hole” and the C_{H}3 constant domain of a second heavy chain comprises a complementary “hole” or “knob,” respectively, so as to permit fitting of a “knob” into a “hole,” thereby, favoring heterodimerization over homodimerization of a mixture of two different immunoglobulin heavy chains. In another embodiment, the complex additionally comprises at least one amino acid change in the C_{H}3 constant domain of the second heavy chain so as to form the complementary “hole” or “knob.”

In one embodiment, the immunoglobulin component and ACE2 component, the immunoglobulin or the immunoglobulin heavy chain of the Fe fragment comprises a Fc heterodimer with functional Fc effector functions. In another embodiment, the bispecific protein complex, the immunoglobulin component and ACE2 component, the immunoglobulin or the immunoglobulin heavy chain of the F_{c} fragment comprises a Fc heterodimer mutated so as to reduce or abolish Fc effector function.

In one embodiment, the Fc effector function is to support binding of Fc receptor and/or complement protein 1q (C1q). In another embodiment, the Fc effector function is antibody-dependent cellular cytotoxicity (ADCC), antibody-dependent cellular phagocytosis (ADCP), complement-dependent cytotoxicity (CDC) or a combination thereof. In another embodiment, the mutated Fc has one or more amino acid change. In a further embodiment, the amino acid change decreases or abolishes binding of the Fc receptor or complement protein 1q (C1q) to an immunoglobulin or immunoglobulin fragment. In another embodiment, the amino acid change decreases or abolishes binding of the Fcγ receptor or complement protein 1q (C1q) to IgG or IgG fragment. In another embodiment, the Fcγ receptor is any of Fcγ receptor 1, Fcγ receptor II and Fcγ receptor III and a combination thereof. In another embodiment, the amino acid change decreases or abolishes antibody-dependent cellular cytotoxicity (ADCC), antibody-dependent cellular phagocytosis (ADCP), complement-dependent cytotoxicity (CDC) or a combination thereof. In another embodiment, the amino acid change is at aspartic acid 265, asparagine 297 or both for IgG or equivalent, wherein equivalent is one or more amino acid change at other amino acid position of IgG reducing or abolishing Fc effector function or at a corresponding position or other position for IgM, IgD, IgA or IgE. In another embodiment, the amino acid change is any of D265A, N297G and both. In another embodiment, the amino acid change is D265A and N297G. In another embodiment, the bispecific protein further lacks or has reduced Fc effector function. In another embodiment, the bispecific protein further comprises D265A and N297G amino acid substitutions in heavy chain constant region. In another embodiment, the bi-specific protein comprises a homodimer of a polypeptide comprising an ACE2 extracellular domain fragment or its variants, a Fc immunoglobulin fragment, and a diabody or scFv. In another embodiment, the polypeptide comprises from the amino-to-carboxyl terminus: the ACE2 extracellular domain fragment or its variants, the Fc immunoglobulin fragment, and a diabody or scFv.

In one embodiment, the ACE2 extracellular domain fragment consists of or comprises amino acid residues 1-614 of SEQ ID NO: 1 or a polypeptide of SEQ ID NO: 3. In another embodiment, the ACE2 extracellular domain fragment additionally has reduced or lacks peptidase or carboxypeptidase activity. In another embodiment, the ACE2 extracellular domain fragment additionally comprises H374N and H378N amino acid substitutions, or alternatively, H374N and H378R amino acid substitutions. In another embodiment, the ACE2 variant increases binding affinity or binding to SARS-CoV-2 virus or SARS-CoV-2 S-protein. In another embodiment, the immunoglobulin fragment, Fc, comprises a hinge region and C_{H}2 and C_{H}3 constant domains of a heavy chain immunoglobulin. In another embodiment, the Fc additionally has reduced or lacks Fc effector function. In another embodiment, the Fe additionally comprises D265A and N297G amino acid substitution.

In another embodiment, the diabody or scFv binds SARS-CoV-2 virus or SARS-CoV-2 S-protein at an antigenic site other than a site bound by ACE2 extracellular domain fragment and does not compete with ACE2 binding. In another embodiment, the diabody or scFv is derived from CR3022 scFv or comprises the CDRs of CR3022 scFv. In another embodiment, one or more peptide linkers may be used to link the ACE2 extracellular domain fragment or its variants, the Fc immunoglobulin fragment, and the diabody or scFv. In another embodiment, the protein is an antibody comprising two identical immunoglobulin heavy chains stabilized by intermolecular disulfide bonds at the hinge region, two identical immunoglobulin light chains with each light chain associated with a heavy chain so as to form a functional antigen-binding determinant and an ACE2 extracellular domain or its fragment, wherein the ACE2 extracellular domain or its fragment, optionally with a signal sequence, is linked to the amino terminus of each heavy chain.

In one embodiment, the protein is an antibody comprising two identical immunoglobulin heavy chains stabilized by intermolecular disulfide bonds at the hinge region, two identical immunoglobulin light chains with each light chain associated with a heavy chain so as to form a functional antigen-binding determinant and an ACE2 extracellular domain or its fragment, wherein the ACE2 extracellular domain or its fragment, optionally with a signal sequence, is linked to the carboxy terminus of each heavy chain. In another embodiment, the protein is an antibody comprising two identical immunoglobulin heavy chains stabilized by intermolecular disulfide bonds at the hinge region, two identical immunoglobulin light chains with each light chain associated with a heavy chain so as to form a functional antigen-binding determinant and an ACE2 extracellular domain or its fragment, wherein the ACE2 extracellular domain or its fragment, optionally with a signal sequence, is linked to the amino terminus of each light chain. In another embodiment, the protein is or comprises a homodimer of an immunoglobulin heavy chain fragment from a Fc immunoglobulin fragment (Fc heavy chain fragment) comprising a hinge region and two constant domains, C_{H}2 and C_{H}3, and an ACE2 extracellular domain or its fragment linked to amino terminus of the Fc heavy chain fragment, and further comprising an immunoglobulin heavy chain fragment from a Fab fragment (Fab heavy chain fragment), a scFv, a diabody or a target protein binding domain linked to carboxyl terminus of the Fc heavy chain fragment, wherein the homodimer comprises two Fc heavy chain fragments held together by disulfide bonds at the hinge region. In another embodiment, the protein is or comprises a homodimer of a polypeptide comprising a first component comprising an immunoglobulin heavy chain fragment from a Fc immunoglobulin fragment (Fc heavy chain fragment) comprising a hinge region and two constant domains, C_{H}2 and C_{H}3, a second component comprising an immunoglobulin heavy chain fragment from a Fab fragment (Fab heavy chain fragment), a scFv, a diabody or a target protein binding domain and a third component an ACE2 extracellular domain or its fragment, wherein the polypeptide comprises from amino-to-carboxyl terminus direction the second component, the first component and the third component, and wherein the homodimer is stabilized by disulfide bonds at the hinge region contained in the Fc heavy chain fragment of the first component.

In one embodiment, the protein is a bispecific protein with two binding specificities formed by a heterodimer comprising or consisting of a first polypeptide comprising an first immunoglobulin heavy chain fragment from a Fc immunoglobulin fragment (first Fe heavy chain fragment) comprising a hinge region and two constant domains, C_{H}2 and C_{H}3, and an immunoglobulin heavy chain fragment from a Fab fragment (Fab heavy chain fragment), a scFv, a diabody or a target protein binding domain linked to amino terminus of the first Fc heavy chain fragment, and a second polypeptide comprising a second immunoglobulin heavy chain fragment from a Fc immunoglobulin fragment (second Fc heavy chain fragment) comprising a hinge region and two constant domains, C_{H}2 and C_{H}3, and an ACE2 extracellular domain or its fragment linked to amino terminus of the second Fc heavy chain fragment, and further wherein heterodimer formation is favored between the first Fc heavy chain fragment and the second Fc heavy chain fragment by the introduction of complementary “knobs” and “holes” in the C_{H}3 constant domain of the two different heavy chain fragments and wherein the heterodimer is stabilized by presence of disulfide bonds between the two hinge regions.

In a further embodiment, the bispecific protein comprises the Fab heavy chain fragment additionally comprises an immunoglobulin light chain, wherein the light chain associates with the first polypeptide so as to form a functional antigenic binding determinant. In another embodiment, the antigenic binding determinant is directed to SARS-CoV-2 virus or SARS-CoV-2 S-protein.

In one embodiment, the protein consists of or comprises an ACE2 signal sequence and extracellular domain fragment from amino acid residue 1 to 740 of SEQ ID NO: 1 linked to amino terminus of a Fc immunoglobulin heavy chain fragment, wherein the Fc immunoglobulin heavy chain fragment comprises a hinge region, and C_{H}2 and C_{H}3 constant domains. In another embodiment, the protein consists of or comprises an ACE2 signal sequence and extracellular domain fragment from amino acid residue 1 to 740 of SEQ ID NO: 1 linked to amino terminus of a Fc immunoglobulin heavy chain fragment, wherein the Fe immunoglobulin heavy chain fragment comprises a hinge region, and C_{H}2 and C_{H}3 constant domains and wherein the Fc further comprises D265A and N297G to reduce or abolish antibody effector function. In another embodiment, the protein has the following amino acid sequence:

(SEQ ID NO: 12)
MSSSSWLLLSLVAVTAAQSTIEEQAKTFLDKFNHEAEDLFYQSSLASWNY
NTNITEENVQNMNNAGDKWSAFLKEQSTLAQMYPLQEIQNLTVKLQLQAL
QQNGSSVLSEDKSKRLNTILNTMSTIYSTGKVCNPDNPQECLLLEPGLNE
IMANSLDYNERLWAWESWRSEVGKQLRPLYEEYVVLKNEMARANHYEDYG
DYWRGDYEVNGVDGYDYSRGQLIEDVEHTFEEIKPLYEHLHAYVRAKLMN
AYPSYISPIGCLPAHLLGDMWGRFWTNLYSLTVPFGQKPNIDVTDAMVDQ
AWDAQRIFKEAEKFFVSVGLPNMTQGFWENSMLTDPGNVQKAVCHPTAWD
LGKGDFRILMCTKVTMDDFLTAHHEMGHIQYDMAYAAQPFLLRNGANEGF
HEAVGEIMSLSAATPKHLKSIGLLSPDFQEDNETEINFLLKQALTIVGTL
PFTYMLEKWRWMVFKGEIPKDQWMKKWWEMKREIVGVVEPVPHDETYCDP
ASLFHVSNDYSFIRYYTRTLYQFQFQEALCQAAKHEGPLHKCDISNSTEA
GQKLFNMLRLGKSEPWTLALENVVGAKNMNVRPLLSYFEPLFTWLKDQNK
NSFVGWSTDWSPYADQSIKVRISLKSALGDKAYEWNDNEMYLFRSSVAYA
MRQYFLKVKNQMILFGEEDVRVANLKPRISFNFFVTAPKNVSDIIPRTEV
EKAIRMSRSRINDAFRLNDNSLEFLGIQPTLGPPNQPPVSDKTHTCPPCP
APELLGGPSVFLFPPKPKDTLMISRTPEVTCVVVAVSHEDPEVKFNWYVD
GVEVHNAKTKPREEQYGSTYRVVSVLTVLHQDWLNGKEYKCKVSNKALPA
PTERTISKAKGQPREPQVYTLPPSREEMTKNQVSLTCLVKGFYPSDIAVE
WESNGQPENNYKTTPPVLDSDGSFFLYSKLTVDKSRWQQGNVFSCSVMHE
ALHNHYTQKSSLSPGK.

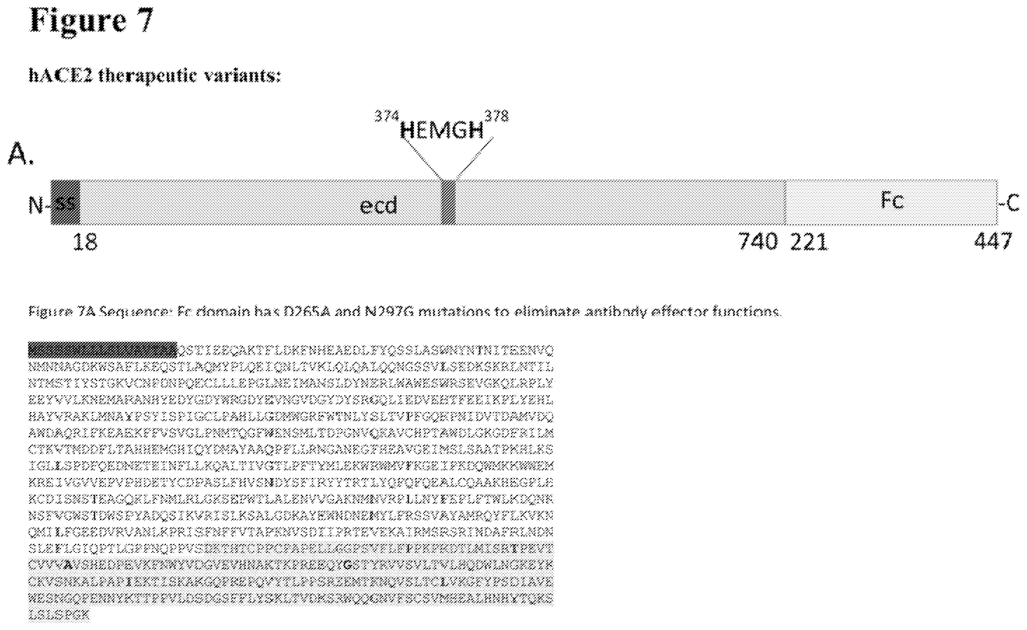

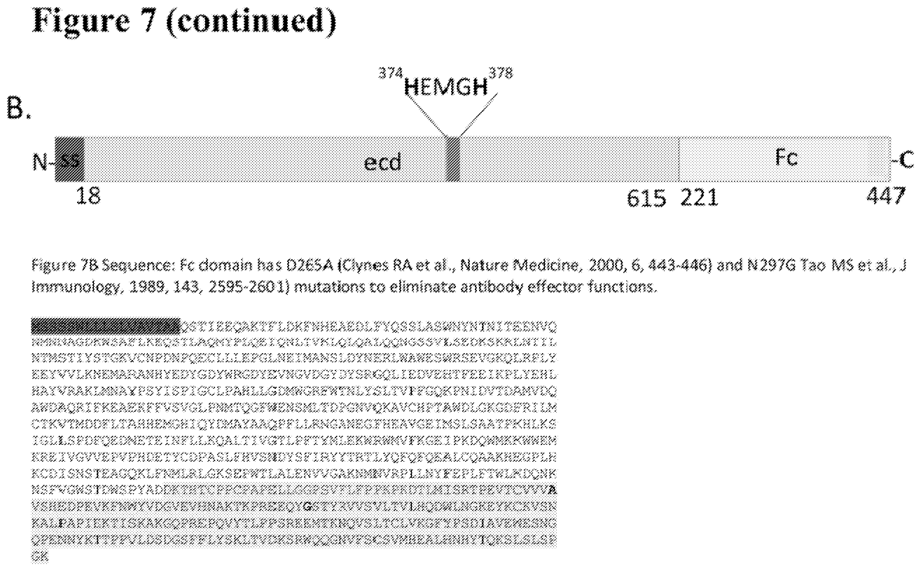

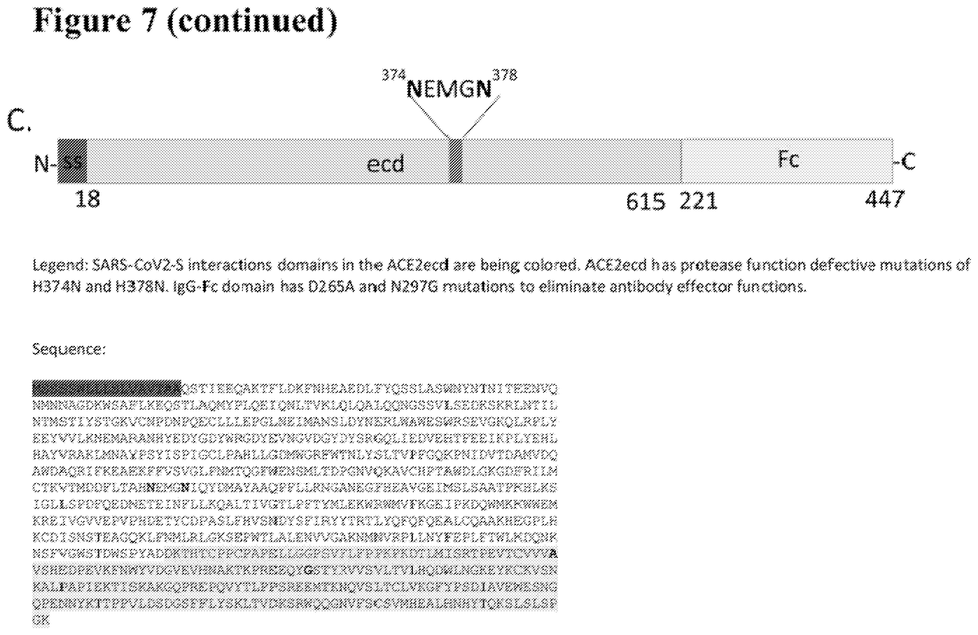

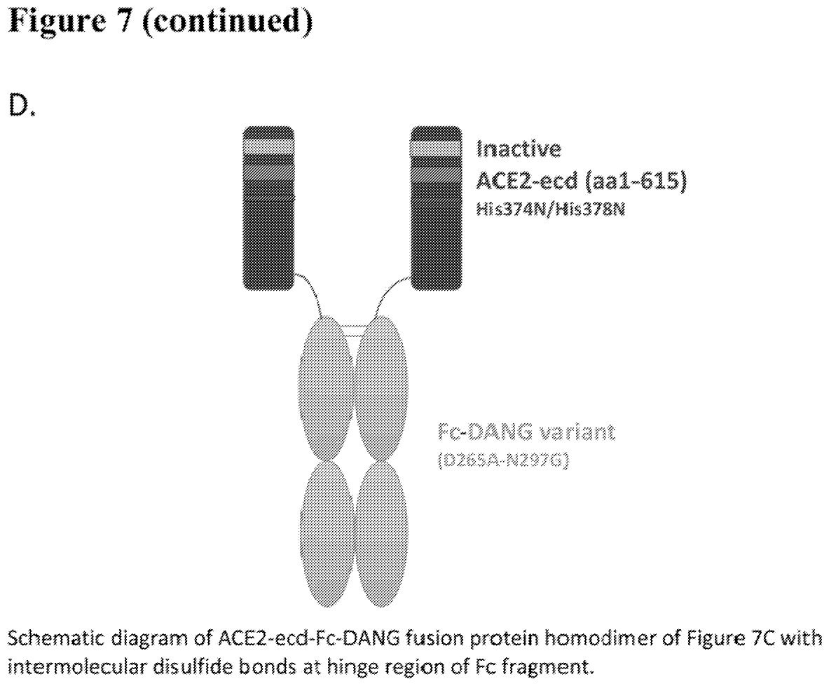

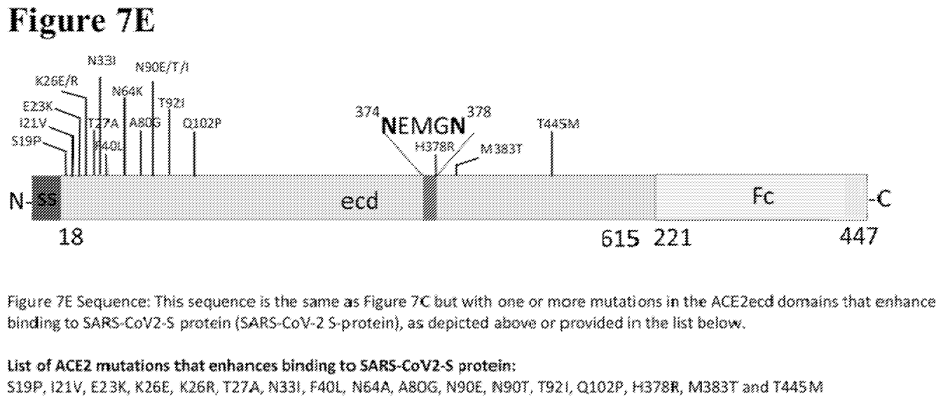

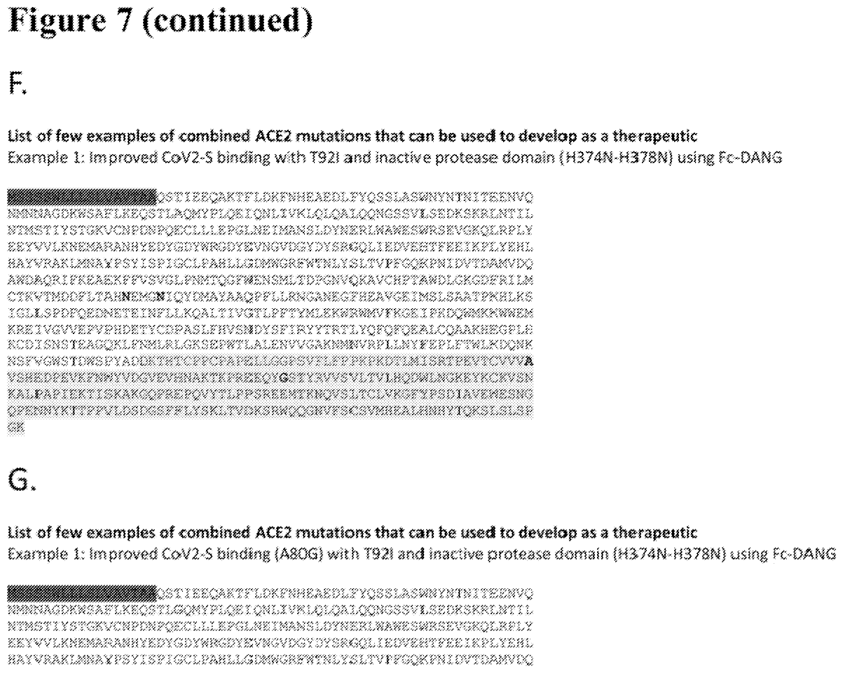

In another embodiment, the protein may include the amino acid sequence as shown in any of FIGS. 7A-C.

In one embodiment, the protein consists of or comprises an ACE2 signal sequence and extracellular domain fragment from amino acid residue 1 to 615 of SEQ ID NO: 1 linked to amino terminus of a Fc immunoglobulin heavy chain fragment, wherein the Fe immunoglobulin heavy chain fragment comprises a hinge region, and C_{H}2 and C_{H}3 constant domains. In another embodiment, the protein consists of or comprises an ACE2 signal sequence and extracellular domain fragment from amino acid residue 1 to 615 of SEQ ID NO: 1 linked to amino terminus of a Fc immunoglobulin heavy chain fragment, wherein the Fc immunoglobulin heavy chain fragment comprises a hinge region, and C_{H}2 and C_{H}3 constant domains and wherein the Fc further comprises D265A and N297G to reduce or abolish antibody effector function. In another embodiment, the protein has the following amino acid sequence:

(SEQ ID NO: 13)
MSSSSWLLLSLVAVTAAQSTIEEQAKTFLDKFNHEAEDLFYQSSLASWNY
NTNITEENVQNMNNAGDKWSAFLKEQSTLAQMYPLQEIQNLTVKLQLQAL
QQNGSSVLSEDKSKRINTILNTMSTIYSTGKVCNPDNPQECLLLEPGLNE
IMANSLDYNERLWAWESWRSEVGKQLRPLYEEYVVLKNEMARANHYEDYG
DYWRGDYEVNGVDGYDYSRGQLLEDVERTFEEIKPLYEHLHAYVRAKIMN
AYPSYISPIGCLPAALLGDMWGREWTNLYSLTVPFGQKPNIDVTDAMVDQ
AWDAQRIFKEAEKFFVSVGLPNMTQGFWENSMLTDPGNVQKAVCHPTAWD
LGKGDFRILMCTKVTMDDFLTAHHEMGHIQYDMAYAAQPFLLRNGANEGF
REAVGEIMSLSAATPKHLKSIGLLSPDFQEDNETEINFLLKQALTIVGTL
PFTYMLEKWRWMVFKGEIPKDQWMKKWWEMKREIVGVVEPVPHDETYCDP
ASLFHVSNDYSFIRYYTRTLYQFQFQEALCQAAKHEGPLHKCDISNSTEA
GQKLFNMLRLGKSEPWTLALENVVGAKNMNVRPLLNYFEPLFTWLKDQNK
NSFVGWSTDWSPYADDKTHTCPPCPAPELLGGPSVFLFPPKPKDTLMISR
TPEVTCVVVAVSHEDPEVKFNWYVDGVEVHNAKTKPREEQYGSTYRVVSV
LTVLHQDWLNGKEYKCKVSNKALPAPIEKTISKAKGQPREPQVYTLPPSR
EEMTKNQVSLTCLVKGFYPSDIAVEWESNGQPENNYKTTPPVLDSDGSFF
LYSKLTVDKSRWQQGNVFSCSVMGEALHNHYTQKSLSLSPGK

In another embodiment, the protein may include the amino acid sequence as shown in any of FIGS. 7A-C.

45) In one embodiment, the ACE2 extracellular domain fragment additional comprises one or more amino acid changes which increases binding or binding affinity of the ACE2 fragment for SARS-CoV-2 virus or SARS-CoV-2 S-protein. In another embodiment, the amino acid changes are at any of S19, 121. E23, K26, K26, T27, N33, F40, N64, A80, N90, T92, Q102, H378, M383 and T445 and a combination thereof. In another embodiment, the amino acid change is any of S19P, I21V, E23K, K26E, K26R, T27A, N33I, F40L, N64K, A800, N90I, N90T, T92I, Q102P, H378R, M383T and T445M and a combination thereof. In another embodiment, the ACE2 extracellular domain fragment additional comprises amino acid changes at S19, K26, T27, N90 and H378. In another embodiment, the amino acid changes are S19P, K26R, T27A, N90I and H378R. In another embodiment, the amino acid changes are S19P, K26R, T27A, N90T and H378R. In another embodiment, the ACE2 extracellular domain fragment additional comprises amino acid changes at S19, K26, T27, T92 and H378. In another embodiment, the amino acid changes are S19P, K26R, T27A, N92I and H378R. In another embodiment, the ACE2 extracellular domain fragment additional comprises amino acid changes at S19, T27 and N90. In another embodiment, the amino acid changes are S19P, T27A and N90I. In another embodiment, the amino acid changes are S19P, T27A and N90T. In another embodiment, the amino acid changes increase binding or binding affinity of the ACE2 fragment for SARS-CoV-2 virus or SARS-CoV-2 S-protein.

In one embodiment, the ACE2 extracellular domain fragment additional comprises amino acid changes to reduce or abolish peptidase or carboxypeptidase activity. In another embodiment, the ACE2 extracellular domain fragment additional comprises amino acid change at H374, H378 or both. Examples of the amino acid change include, but are not limited to, H374N, H378N, H378R, both H374N and H378N, and both H374N and H378R.

In another embodiment, the ACE2 extracellular domain fragment additional comprises either both H374N and H378N or both H374N and H378R amino acid substitution. In another embodiment, the protein consists of or comprises an ACE2 signal sequence and extracellular domain fragment from amino acid residue 1 to 615 of SEQ ID NO: 1 linked to amino terminus of a Fc immunoglobulin heavy chain fragment, wherein the Fc immunoglobulin heavy chain fragment comprises a hinge region, and C_{H}2 and C_{H}3 constant domains, wherein the Fc fragment additionally comprises D265A and N297G amino acid substitutions, and wherein the ACE2 fragment additionally comprises H374N and H378N amino acid substitutions.

In one embodiment, the protein has the following amino acid sequence:

(SEQ ID NO: 14)
MSSSSWLLLSLVAVTAAQSTIEEQAKTFLDKFNHEAEDLFYQSSLASWNY
NTNITEENVQNMNNAGDKWSAFLKEQSTLAQMYPLQEIQNLTVKLQLQAL
QQNGSSVLSEDKSKRINTILNTMSTIYSTGKVCNPDNPQECLLLEPGLNE
IMANSLDYNERLWAWESWRSEVGKQLRPLYEEYVVLKNEMARANHYEDYG
DYWRGDYEVNGVDGYDYSRGQLLEDVERTFEEIKPLYEHLHAYVRAKIMN
AYPSYISPIGCLPAALLGDMWGREWTNLYSLTVPFGQKPNIDVTDAMVDQ
AWDAQRIFKEAEKFFVSVGLPNMTQGFWENSMLTDPGNVQKAVCHPTAWD
LGKGDFRILMCTKVTMDDFLTAHHEMGHIQYDMAYAAQPFLLRNGANEGF
REAVGEIMSLSAATPKHLKSIGLLSPDFQEDNETEINFLLKQALTIVGTL
PFTYMLEKWRWMVFKGEIPKDQWMKKWWEMKREIVGVVEPVPHDETYCDP
ASLFHVSNDYSFIRYYTRTLYQFQFQEALCQAAKHEGPLHKCDISNSTEA
GQKLFNMLRLGKSEPWTLALENVVGAKNMNVRPLLNYFEPLFTWLKDQNK
NSFVGWSTDWSPYADDKTHTCPPCPAPELLGGPSVFLFPPKPKDTLMISR
TPEVTCVVVAVSHEDPEVKFNWYVDGVEVHNAKTKPREEQYGSTYRVVSV
LTVLHQDWLNGKEYKCKVSNKALPAPIEKTISKAKGQPREPQVYTLPPSR
EEMTKNQVSLTCLVKGFYPSDIAVEWESNGQPENNYKTTPPVLDSDGSFF
LYSKLTVDKSRWQQGNVFSCSVMGEALHNHYTQKSLSLSPGK

In another embodiment, the protein may include the amino acid sequence as shown in any of FIGS. 7A-C, and FIGS. 7F-H.

In one embodiment, the protein consists of or comprises an ACE2 signal sequence and extracellular domain fragment from amino acid residue 1 to 615 of SEQ ID NO: 1 linked to amino terminus of a Fc immunoglobulin heavy chain fragment, wherein the Fc immunoglobulin heavy chain fragment comprises a hinge region, and C_{H}2 and C_{H}3 constant domains and wherein the ACE2 fragment additionally comprises one or more amino acid changes selected from the group consisting of S19P, I21V, E23K, K26E, K26R, T27A, N33I, F40L, N64K, A80G, N90I, N90T, T92I, Q102P, H378R, M383T and T445M and a combination thereof. In another embodiment, the protein consists of or comprises an ACE2 signal sequence and extracellular domain fragment from amino acid residue 1 to 615 of SEQ ID NO: 1 linked to amino terminus of a Fc immunoglobulin heavy chain fragment, wherein the Fc immunoglobulin heavy chain fragment comprises a hinge region, and C_{H}2 and C_{H}3 constant domains, wherein the Fe fragment additionally comprises D265A and N297G amino acid substitutions, and wherein the ACE2 fragment additionally comprises one or more amino acid changes selected from the group consisting of S19P, I21V, E23K, K26E, K26R, T27A, N33I, F40L, N64K, A80G, N90I, N90T, T92I, Q102P, H378R, M383T and T445M and a combination thereof. In another embodiment, the protein consists of or comprises an ACE2 signal sequence and extracellular domain fragment from amino acid residue 1 to 615 of SEQ ID NO: 1 linked to amino terminus of a Fc immunoglobulin heavy chain fragment, wherein the Fe immunoglobulin heavy chain fragment comprises a hinge region, and C_{H}2 and C_{H}3 constant domains, wherein the ACE2 fragment additionally comprises one or more amino acid changes selected from the group consisting of S19P, I21V, E23K, K26E, K26R, T27A, N33I, F40L, N64K, A80G, N90I, N90T, T92L, Q102P, H378R, M383T and T445M and a combination thereof, and wherein the ACE2 fragment additionally comprises H374N and either H378N or N378R amino acid substitutions.

In another embodiment, the protein consists of or comprises an ACE2 signal sequence and extracellular domain fragment from amino acid residue 1 to 615 of SEQ ID NO: 1 linked to amino terminus of a Fc immunoglobulin heavy chain fragment, wherein the Fc immunoglobulin heavy chain fragment comprises a hinge region, and C_{H}2 and C_{H}3 constant domains, wherein the Fc fragment additionally comprises D265A and N297G amino acid substitutions, wherein the ACE2 fragment additionally comprises one or more amino acid changes selected from the group consisting of S19P, I21V, E23K, K26E, K26R, T27A, N33I, F40L, N64K, A80G, N90I, N90T, T92L, Q102P, H378R, M383T and T445M and a combination thereof, and wherein the ACE2 fragment additionally comprises H374N and either H378N or H378R amino acid substitutions.

In another embodiment, the protein consists of or comprises an ACE2 signal sequence and extracellular domain fragment from amino acid residue 1 to 615 of SEQ ID NO: 1 linked to amino terminus of a Fc immunoglobulin heavy chain fragment, wherein the Fe immunoglobulin heavy chain fragment comprises a hinge region, and C_{H}2 and C_{H}3 constant domains, and wherein the ACE2 fragment additionally comprises S19P, K26R, T27A, N33I, A80G, N90I, T92I and H378R amino acid substitutions. In another embodiment, the protein consists of or comprises an ACE2 signal sequence and extracellular domain fragment from amino acid residue 1 to 615 of SEQ ID NO: 1 linked to amino terminus of a Fc immunoglobulin heavy chain fragment, wherein the Fc immunoglobulin heavy chain fragment comprises a hinge region, and C_{H}2 and C_{H}3 constant domains, wherein the Fc fragment additionally comprises D265A and N297G amino acid substitutions, and wherein the ACE2 fragment additionally comprises S19P, K26R, T27A, N33I, A80G, N90I, T92I and H378R amino acid substitutions. In another embodiment, the protein consists of or comprises an ACE2 signal sequence and extracellular domain fragment from amino acid residue 1 to 615 of SEQ ID NO: 1 linked to amino terminus of a Fc immunoglobulin heavy chain fragment, wherein the Fc immunoglobulin heavy chain fragment comprises a hinge region, and C_{H}2 and C_{H}3 constant domains, wherein the ACE2 fragment additionally comprises S19P, K26R, T27A, N33I, A80G, N90I, T92I and 1-1378R amino acid substitutions, and wherein the ACE2 fragment additionally comprises H374N amino acid substitutions. In another embodiment, the protein consists of or comprises an ACE2 signal sequence and extracellular domain fragment from amino acid residue 1 to 615 of SEQ ID NO: 1 linked to amino terminus of a Fc immunoglobulin heavy chain fragment, wherein the Fe immunoglobulin heavy chain fragment comprises a hinge region, and C_{H}2 and C_{H}3 constant domains, wherein the Fc fragment additionally comprises D265A and N297G amino acid substitutions, wherein the ACE2 fragment additionally comprises S19P, K26R, T27A, N33I, A80G, N90I, T92I and H378R amino acid substitutions, and wherein the ACE2 fragment additionally comprises H374N amino acid substitutions.

In another embodiment, the protein has the following amino acid sequence:

(SEQ ID NO: 15)
MSSSSWLLSLVAVTAAQPTIEEQARAFLDKFNHEAEDLFYQSSLASWNYN
TNITEENVQNMNNAGDKWSAFLKEQSTLAQMYPLQEIQILTVKLQLQALQ
QNGSSVLSEDKSKRLNTILNTMSTIYSTGKVCNPDNPQECLLLEPGLNEI
MANSLDYNERLWAWESWRSEVGKQLRPLYEEYVVLKNEMAPANHYEDYGD
YWRGDYEVNGVDGYDYSRGQLIEDVEHTFEEIKPLYEHLHAYVRAKLMNA
YPSYISPIGCLPAHLLGDMWGRFWTNLYSLTVPFGQKPNIDVTDAMVDQA
WDAQRIFKEAEKFFVSVGLPNMTQGFWENSMLTDPGNVQKAVCHPTAWDL
GKGDFRILMCTKVTMDDFLTAHNEMGRIQYDMAYAAQPFLLRNGANEGFH
EAVGEIMSLSAATPKHLKSIGLLSPDFQEDNETEINFLLKQALTIVGTLP
FTYMLEKWRWMVFKGEIPKDQWMKKWWEMKREIVGVVEPVPHDETYCDPA
SLFHVSNDYSFIRYYTRTLYQFQFQEALCQAAKHEGPLHKCDISNSTEAG
QKLFNMLRLGKSEPWTLALENVVGAKNMNVRPLLNYFEPLFTWLKDQNKN
SFVGWSTDWSPYADDKTHTCPPCPAPELLGGPSVFLFPPKPKDTLMISRT
PEVTCVVVAVSHEDPEVKFNWYVDGVEVHNAKTKPREEQYGSTYRVVSVL
TVLHQDWLNGKEYKCKVSNKALPAPIEKTISKAKGQPREPQVYTLPPSRE
EMTKNQVSLTCLVKGFYPSDIAVEWESNGQPENNYKTTPPVLDSDGSFFL
YSKLTVDKSRWQQGNVFSCSVMHEALHNHYTQKSLSLSPGK.

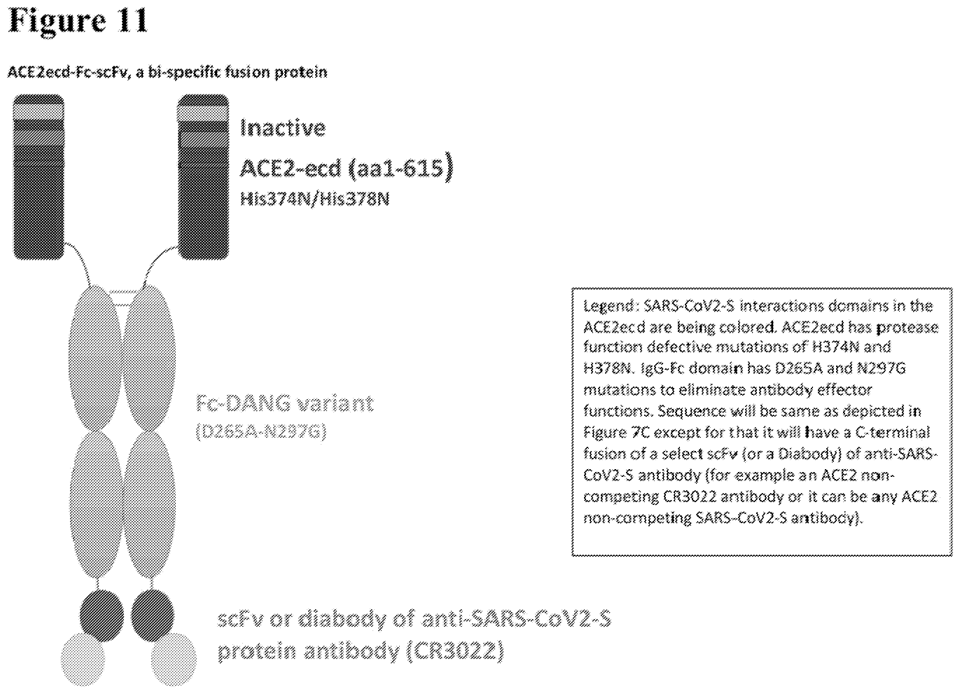

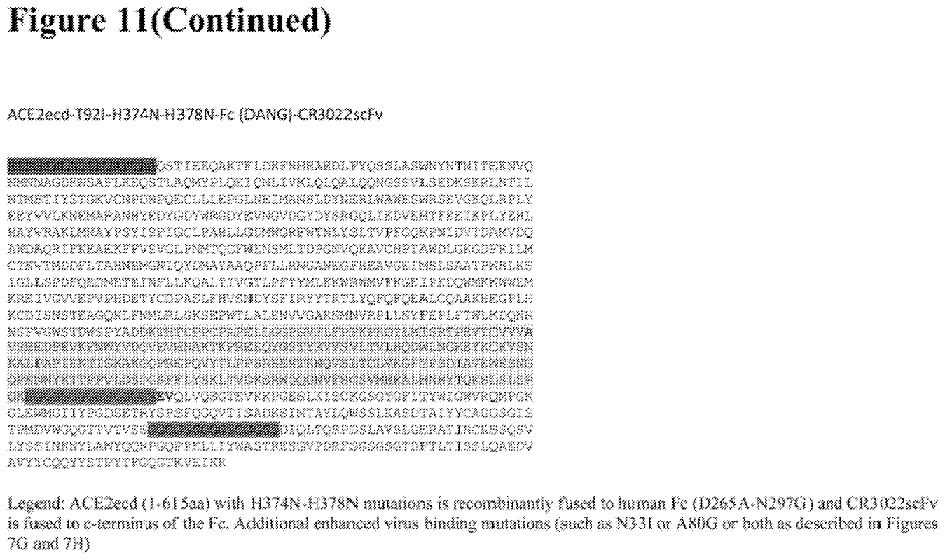

In another embodiment, the protein may include the amino acid sequence as shown in any of FIGS. 7F-H, and FIG. 11.

In another embodiment, the protein consists of or comprises an ACE2 signal sequence and extracellular domain fragment from amino acid residue 1 to 615 of SEQ ID NO: 1 linked to amino terminus of a Fc immunoglobulin heavy chain fragment, wherein the Fc immunoglobulin heavy chain fragment comprises a hinge region, and C_{H}2 and C_{H}3 constant domains, and wherein the ACE2 fragment additionally comprises S19P, K26R, T27A, N33I, A80G, N90I, T92I and H378R amino acid substitutions. In another embodiment, the protein consists of or comprises an ACE2 signal sequence and extracellular domain fragment from amino acid residue 1 to 615 of SEQ ID NO: 1 linked to amino terminus of a Fe immunoglobulin heavy chain fragment, wherein the Fc immunoglobulin heavy chain fragment comprises a hinge region, and C_{H}2 and C_{H}3 constant domains, wherein the Fc fragment additionally comprises D265A and N297G amino acid substitutions, and wherein the ACE2 fragment additionally comprises S19P, K26R, T27A, N33L, A80G, N90I, T92I and H378R amino acid substitutions. In another embodiment, the protein consists of or comprises an ACE2 signal sequence and extracellular domain fragment from amino acid residue 1 to 615 of SEQ ID NO: 1 linked to amino terminus of a Fc immunoglobulin heavy chain fragment, wherein the Fc immunoglobulin heavy chain fragment comprises a hinge region, and C_{H}2 and C_{H}3 constant domains, wherein the ACE2 fragment additionally comprises S19P, K26R, T27A, N33I, A800, N90I, T92I and H378R amino acid substitutions, and wherein the ACE2 fragment additionally comprises H374N amino acid substitutions. In another embodiment, the protein consists of or comprises an ACE2 signal sequence and extracellular domain fragment from amino acid residue 1 to 615 of SEQ ID NO: 1 linked to amino terminus of a Fe immunoglobulin heavy chain fragment, wherein the Fc immunoglobulin heavy chain fragment comprises a hinge region, and C_{H}2 and C_{H}3 constant domains, wherein the Fc fragment additionally comprises D265A and N297G amino acid substitutions, wherein the ACE2 fragment additionally comprises S19P, K26R, T27A, N33I, A80G, N90I, T92I and H378R amino acid substitutions, and wherein the ACE2 fragment additionally comprises H374N amino acid substitutions.

In one embodiment, the protein has the following amino acid sequence:

(SEQ ID NO: 16)
MSSSSWLLLSLVAVTAAQPTIEEQARAFLDKFNHEAEDLFYQSSLASWNY
NTNITEENVQNMNNAGDKWSAFLKEQSTLAQMYPLQEIQTLTVKLQLQAL
QQNGSSVLSEDKSKRLNTILNTMSTIYSTGKVCNPDNPQECLLLEPGLNE
IMANSLDYNERLWAWESWRSEVGKQLRPLYEEYVVLKNEMARANHYEDYG
DYWPGDYEVNGVDGYDYSRGQLIEDVEHTFEEIKPLYEHLHAYVRAKLMN
AYPSYISPIGCLPAHLLGDMWGRFWTNLYSLTVPFGQKPNIDVTDAMVDQ
AWDAQRIFKEAEKFFVSVGLPNMTQGFWENSMLTDPGNVQKAVCHPTAWD
LGKGDFRILMCTKVTMDDFLTAHNEMGRIQYDMAYAAQPFLLRNGANEGF
HEAVGEIMSLSAATPKHLKSIGLLSPDFQEDNETEINFLLKQALTIVGTL
PFTYMLEKWRWMVFKGEIPKDQWMKKWWEMKREIVGVVEPVPHDETYCDP
ASLFHVSNDYSFIRYYTRTLYQFQFQEALCQAAKHEGPLHKCDISNSTEA
GQKLFNMLRLGKSEPWTLALENVVGAKNMNVRPLLNYFEPLFTWLKDQNK
NSFVGWSTDWSPYADDKTHTCPPCPAPELLGGPSVFLFPPKPKDTLMISR
TPEVTCVVVAVSHEDPEVKFNWYVDGVEVHNAKTKPREEQYGSTYRVVSV
LTVLHQDWLNGKEYKCKVSNKALPAPIEKTISKAKGQPREPQVYTLPPSR
EEMTKNQVSLTCLVKGFYPSDIAVEWESNGQPENNYKTTPPVLDSDGSFF
LYSKLTVDKSRWQQGNVFSCSVMHEALHNHYTQKSLSLSPGK.

In another embodiment, the protein may include the amino acid sequence as shown in any of FIGS. 7A-C, FIGS. 7F-H, and FIG. 11.

In one embodiment, the protein consists of or comprises an ACE2 signal sequence and extracellular domain fragment from amino acid residue 1 to 615 of SEQ ID NO: 1 linked to amino terminus of a Fc immunoglobulin heavy chain fragment, wherein the Fc immunoglobulin heavy chain fragment comprises a hinge region, and C_{H}2 and C_{H}3 constant domains, and wherein the ACE2 fragment additionally comprises S19P, K26R, T27A, N33I, A80G, N90I, T92I and H378R amino acid substitutions. In another embodiment, the protein consists of or comprises an ACE2 signal sequence and extracellular domain fragment from amino acid residue 1 to 615 of SEQ ID NO: 1 linked to amino terminus of a Fc immunoglobulin heavy chain fragment, wherein the Fc immunoglobulin heavy chain fragment comprises a hinge region, and C_{H}2 and C_{H}3 constant domains, wherein the Fc fragment additionally comprises D265A and N297G amino acid substitutions, and wherein the ACE2 fragment additionally comprises S19P, K26R, T27A, N33I, A80G, N90I, T92I and H378R amino acid substitutions. In another embodiment, the protein consists of or comprises an ACE2 signal sequence and extracellular domain fragment from amino acid residue 1 to 615 of SEQ ID NO: 1 linked to amino terminus of a Fc immunoglobulin heavy chain fragment, wherein the Fc immunoglobulin heavy chain fragment comprises a hinge region, and C_{H}2 and C_{H}3 constant domains, wherein the ACE2 fragment additionally comprises S19P, K26R, T27A, N33I, A80G, N90I, T92I and H378R amino acid substitutions, and wherein the ACE2 fragment additionally comprises H374N amino acid substitutions.

In another embodiment, the protein consists of or comprises an ACE2 signal sequence and extracellular domain fragment from amino acid residue 1 to 615 of SEQ ID NO: 1 linked to amino terminus of a Fc immunoglobulin heavy chain fragment, wherein the Fe immunoglobulin heavy chain fragment comprises a hinge region, and C_{H}12 and C_{H}3 constant domains, wherein the Fc fragment additionally comprises D265A and N297G amino acid substitutions, wherein the ACE2 fragment additionally comprises S19P, K26R, T27A, N33I, A80G, N90I, T92I and H378R amino acid substitutions, and wherein the ACE2 fragment additionally comprises H374N amino acid substitutions.

In another embodiment, the protein consists of or comprises an ACE2 signal sequence and extracellular domain fragment from amino acid residue 1 to 615 of SEQ ID NO: 1 linked to amino terminus of a Fc immunoglobulin heavy chain fragment, wherein the Fc immunoglobulin heavy chain fragment comprises a hinge region, and C_{H}2 and C_{H}3 constant domains, wherein the Fc fragment additionally comprises D265A and N297G amino acid substitutions, wherein the ACE2 fragment additionally optionally comprises S19P, K26R, T27A, N33I, or N33I, A80G, and T92I and I378R amino acid substitutions, and wherein the ACE2 fragment additionally comprises H374N amino acid substitutions.

In another embodiment, the protein has the following amino acid sequence:

(SEQ ID NO: 17)
MSSSSWLLLSLVAVTAAQPTIEEQARAFLDKFNHEAEDLFYQSSLASWNY
NTNITEENVQNMNNAGDKWSAFLKEQSTLAQMYPLQEIQTLIVKLQLQAL
QQNGSSVLSEDKSKRLNTILNTMSTIYSTGKVCNPDNPQECLLLEPGLNE
IMANSLDYNERLWAWESWRSEVGKQLRPLYEEYVVLKNEMARANHYEDYG
DYWPGDYEVNGVDGYDYSRGQLIEDVEHTFEEIKPLYEHLHAYVRAKLMN
AYPSYISPIGCLPAHLLGDMWGRFWTNLYSLTVPFGQKPNIDVTDAMVDQ
AWDAQRIFKEAEKFFVSVGLPNMTQGFWENSMLTDPGNVQKAVCHPTAWD
LGKGDFRILMCTKVTMDDFLTAHNEMGRIQYDMAYAAQPFLLRNGANEGF
HEAVGEIMSLSAATPKHLKSIGLLSPDFQEDNETEINFLLKQALTIVGTL
PFTYMLEKWRWMVFKGEIPKDQWMKKWWEMKREIVGVVEPVPHDETYCDP
ASLFHVSNDYSFIRYYTRTLYQFQFQEALCQAAKHEGPLHKCDISNSTEA
GQKLFNMLRLGKSEPWTLALENVVGAKNMNVRPLLNYFEPLFTWLKDQNK
NSFVGWSTDWSPYADDKTHTCPPCPAPELLGGPSVFLFPPKPKDTLMISR
TPEVTCVVVAVSHEDPEVKFNWYVDGVEVHNAKTKPREEQYGSTYRVVSV
LTVLHQDWLNGKEYKCKVSNKALPAPIEKTISKAKGQPREPQVYTLPPSR
EEMTKNQVSLTCLVKGFYPSDIAVEWESNGQPENNYKTTPPVLDSDGSFF
LYSKLTVDKSRWQQGNVFSCSVMHEALHNHYTQKSLSLSPGK.

In another embodiment, the protein may include the amino acid sequence as shown in any of FIGS. 7A-C, FIGS. 7F-H, and FIG. 11.

In another embodiment, the protein consists of or comprises an ACE2 signal sequence and extracellular domain fragment from amino acid residue 1 to 615 of SEQ ID NO: 1 linked to amino terminus of a Fc immunoglobulin heavy chain fragment, wherein the Fc immunoglobulin heavy chain fragment comprises a hinge region, and C_{H}2 and C_{H}3 constant domains, and wherein the ACE2 fragment additionally comprises S19P, T27A and N90I amino acid substitutions. In another embodiment, the protein consists of or comprises an ACE2 signal sequence and extracellular domain fragment from amino acid residue 1 to 615 of SEQ ID NO: 1 linked to amino terminus of a Fe immunoglobulin heavy chain fragment, wherein the Fc immunoglobulin heavy chain fragment comprises a hinge region, and C_{H}2 and C_{H}3 constant domains, wherein the Fc fragment additionally comprises D265A and N297G amino acid substitutions, and wherein the ACE2 fragment additionally comprises S19P, T27A and N90I amino acid substitutions. In another embodiment, the protein consists of or comprises an ACE2 signal sequence and extracellular domain fragment from amino acid residue 1 to 615 of SEQ ID NO: 1 linked to amino terminus of a Fe immunoglobulin heavy chain fragment, wherein the Fc immunoglobulin heavy chain fragment comprises a hinge region, and C_{H}2 and C_{H}3 constant domains, wherein the ACE2 fragment additionally comprises S19P, T27A and N90I amino acid substitutions, and wherein the ACE2 fragment additionally comprises H374N and H378N amino acid substitutions. In another embodiment, the protein consists of or comprises an ACE2 signal sequence and extracellular domain fragment from amino acid residue 1 to 615 of SEQ ID NO: 1 linked to amino terminus of a Fc immunoglobulin heavy chain fragment, wherein the Fc immunoglobulin heavy chain fragment comprises a hinge region, and C_{H}2 and C_{H}3 constant domains, wherein the Fc fragment additionally comprises D265A and N297G amino acid substitutions, wherein the ACE2 fragment additionally comprises S19P, T27A and N90I amino acid substitutions, and wherein the ACE2 fragment additionally comprises H374N and H378N amino acid substitutions.

In one embodiment the protein has the following amino acid sequence:

(SEQ ID NO: 18)
MSSSSWLLLSLVAVTAAQPTIEEQAKAFLDKFNHEAEDLFYQSSLASWNY
NTNITEENVQNMNNAGDKWSAFLKEQSTLAQMYPLQEIQILTVKLQLQAL
QQNGSSVLSEDKSKRLNTILNTMSTIYSTGKVCNPDNPQECLLLEPGLNE
IMANSLDYNERLWAWESWRSEVGKQLRPLYEEYVVLKNEMARANHYEDYG
DYWRGDYEVNGVDGYDYSRGQLIEDVEHTFEEIKPLYEHLHAYVRAKLMN
AYPSYISPIGCLPAHLLGDMWGRFWTNLYSLTVPFGQKPNIDVTDAMVDQ
AWDAQRIFKEAEKFFVSVGLPNMTQGFWENSMLTDPGNVQKAVCHPTAWD
LGKGDFRILMCTKVTMDDFLTAHNEMGNIQYDMAYAAQPFLLRNGANEGF
HEAVGEIMSLSAATPKHLKSIGLLSPDFQEDNETEINFLLKQALTIVGTL
PFTYMLEKWRWMVFKGEIPKDQWMKKWWEMKREIVGVVEPVPHDETYCDP
ASLFHVSNDYSFIRYYTRTLYQFQFQEALCQAAKHEGPLHKCDISNSTEA
GQKLFNMLRLGKSEPWTLALENVVGAKNMNVRPLLNYFEPLFTWLKDQNK
NSFVGWSTDWSPYADDKTHTCPPCPAPELLGGPSVFLFPPKPKDTLMISR
TPEVTCVVVAVSHEDPEVKFNWYVDGVEVHNAKTKPREEQYGSTYRVVSV
LTVLHQDWLNGKEYKCKVSNKALPAPIEKTISKAKGQPREPQVYTLPPSR
EEMTKNQVSLTCLVKGFYPSDIAVEWESNGQPENNYKTTPPVLDSDGSFF
LYSKLTVDKSRWQQGNVPSCSVMHEALHNHYTQKSLSLSPGK.

In another embodiment, the protein may include the amino acid sequence as shown in any of FIGS. 7F-H, and FIG. 11.

In one embodiment, the protein consists of or comprises an ACE2 signal sequence and extracellular domain fragment from amino acid residue 1 to 615 of SEQ ID NO: 1 linked to amino terminus of a Fc immunoglobulin heavy chain fragment, wherein the Fc immunoglobulin heavy chain fragment comprises a hinge region, and C_{H}2 and C_{H}3 constant domains, and wherein the ACE2 fragment additionally comprises S19P, T27A and N90T amino acid substitutions. In another embodiment, the protein consists of or comprises an ACE2 signal sequence and extracellular domain fragment from amino acid residue 1 to 615 of SEQ ID NO: 1 linked to amino terminus of a Fe immunoglobulin heavy chain fragment, wherein the Fc immunoglobulin heavy chain fragment comprises a hinge region, and C_{H}2 and C_{H}3 constant domains, wherein the Fc fragment additionally comprises D265A and N297G amino acid substitutions, and wherein the ACE2 fragment additionally comprises S19P, T27A and N90T amino acid substitutions. In another embodiment, the protein consists of or comprises an ACE2 signal sequence and extracellular domain fragment from amino acid residue 1 to 615 of SEQ ID NO: 1 linked to amino terminus of a Fe immunoglobulin heavy chain fragment, wherein the Fc immunoglobulin heavy chain fragment comprises a hinge region, and C_{H}2 and C_{H}3 constant domains, wherein the ACE2 fragment additionally comprises S19P, T27A and N90T amino acid substitutions, and wherein the ACE2 fragment additionally comprises H374N and H378N amino acid substitutions. In another embodiment, the protein consists of or comprises an ACE2 signal sequence and extracellular domain fragment from amino acid residue 1 to 615 of SEQ ID NO: 1 linked to amino terminus of a Fc immunoglobulin heavy chain fragment, wherein the Fc immunoglobulin heavy chain fragment comprises a hinge region, and C_{H}2 and C_{H}3 constant domains, wherein the Fc fragment additionally comprises D265A and N297G amino acid substitutions, wherein the ACE2 fragment additionally comprises S19P, T27A and N90T amino acid substitutions, and wherein the ACE2 fragment additionally comprises H374N and H378N amino acid substitutions.

In another embodiment, the protein has the following amino acid sequence:

(SEQ ID NO: 19)
MSSSSWLLLSLVAVTAAQPTIEEQAKAFLDKFNHEAEDLFYQSSLASWNY
NTNITEENVQNMNNAGDKWSAFLKEQSTLAQMYPLQEIQTLTVKLQLQAL
QQNGSSVLSEDKSKRLNTILNTMSTIYSTGKVCNPDNPQECLLLEPGLNE
IMANSLDYNERLWAWESWRSEVGKQLRPLYEEYVVLKNEMARANHYEDYG
DYWRGDYEVNGVDGYDYSRGQLIEDVEHTFEEIKPLYEHLHAYVRAKLMN
AYPSYISPIGCLPAHLLGDMWGRFWTNLYSLTVPFGQKPNIDVTDAMVDQ
AWDAQRIFKEAEKFFVSVGLPNMTQGFWENSMLTDPGNVQKAVCHPTAWD
LGKGDFRILMCTKVTMDDFLTAHNEMGNIQYDMAYAAQPFLLRNGANEGF
HEAVGEIMSLSAATPKHLKSIGLLSPDFQEDNETEINFLLKQALTIVGTL
PFTYMLEKWRWMVFKGEIPKDQWMKKWWEMKREIVGVVEPVPHDETYCDP
ASLFHVSNDYSFIRYYTRTLYQFQFQEALCQAAKHEGPLHKCDISNSTEA
GQKLFNMLRLGKSEPWTLALENVVGAKNMNVRPLLNYFEPLFTWLKDQNK
NSFVGWSTDWSPYADDKTHTCPPCPAPELLGGPSVFLFPPKPKDTLMISR
TPEVTCVVVAVSHEDPEVKFNWYVDGVEVHNAKTKPREEQYGSTYRVVSV
LTVLHQDWLNGKEYKCKVSNKALPAPIEKTISKAKGQPREPQVYTLPPSR
EEMTKNQVSLTCLVKGFYPSDIAVEWESNGQPENNYKTTPPVLDSDGSFF
LYSKLTVDKSRWQQGNVPSCSVMHEALHNHYTQKSLSLSPGK.

In another embodiment, the protein may include the amino acid sequence as shown in any of FIGS. 7F-H, and FIG. 11.

In another embodiment, the immunoglobulin is human or humanized. In another embodiment, the ACE2 fragment is a fragment of human ACE2 protein. In another embodiment, the protein is a homodimer comprising intermolecular disulfide bonds at the hinge region of two polypeptide chains derived from the Fc immunoglobulin heavy chain fragment. In another embodiment, the homodimer is mono-specific. In another embodiment, the homodimer is bivalent. In another embodiment, the protein comprises a synthetic binding domain comprising a combination of segmented ACE2 protein secondary structural motifs and a Fc immunoglobulin fragment, wherein the segmented ACE2 protein secondary structural motifs are ACE2 helix 2 peptide as provided in SEQ ID NO: 6, ACE2 helix 1 peptide as provided in SEQ ID NO: 7 and ACE2 beta turn peptide as provided in SEQ ID NO: 8, wherein the structural motifs are linked in the order from amino-to-carboxyl direction, ACE2 helix 2 peptide-ACE2 helix 1 peptide-ACE2 beta turn peptide and linked by glycine containing linkers to form helix 2-helix 1-beta turn structure (HHB), wherein the Fc fragment comprises an immunoglobulin heavy chain constant region fragment comprising a hinge region and C_{H}2 and C_{H}3 constant domains, and wherein the HHB synthetic binding domain is linked to amino terminus of the Fc fragment to form HHB-Fc hybrid protein. In a further embodiment, the HHB synthetic binding domain binds SARS-CoV-2 virus or SARS-CoV-2 S-protein. In another embodiment, the HHB-Fc hybrid protein forms a homodimer stabilized by intermolecular disulfide bonds at the hinge region of two polypeptide chains. In another embodiment, the homodimer is mono-specific but bivalent. In another embodiment, the protein comprises a synthetic binding domain comprising a combination of segmented ACE2 protein secondary structural motifs and a Fc immunoglobulin fragment, wherein the segmented ACE2 protein secondary structural motifs are ACE2 helix 2 peptide as provided in SEQ ID NO: 6, ACE2 helix 1 peptide as provided in SEQ ID NO: 7 and ACE2 beta turn peptide as provided in SEQ ID NO: 8, wherein the structural motifs are linked in the order from amino-to-carboxyl direction, ACE2 helix 2 peptide-ACE2 helix 1 peptide-ACE2 beta turn peptide and linked by glycine containing linkers to form helix 2-helix 1-beta turn structure (HHB), wherein the Fc fragment comprises an immunoglobulin heavy chain constant region fragment comprising a hinge region and C_{H}2 and C_{H}3 constant domains, wherein the Fc fragment further comprises D265A and N297G amino acid substitutions reducing or abolishing Fc effector function, and wherein the H4B synthetic binding domain is linked to amino terminus of the Fc fragment to form HHB-Fc DANG hybrid protein.

In another embodiment, the HHB-Fc DANG hybrid protein consists of or comprises an amino acid sequence as shown:

(SEQ ID NO: 20)
GTEENVQNMNNAGDKWSAFTKEQSTLAQMYGGEEQAKTFLDKFNHEAEDL
FYQSSLASWNYNTGGGGSGGAWDLGKGDFR DKTHTCPPCPAPELLGGPS
VFLFPPKPKDTLMISRTPEVTCVVVAVSHEDPEVKFNWYVDGVEVHNAKT
KPREEQYGSTYRVVSVLTVLHQDWLNGKEYKCKVSNKALPAPIEKTISKA
KGQPREPQVYTLPPSREEMTKNQVSLTCLVKGFYPSDIAVEWESNGQPEN
NYKTTPPVLDSDGSFFLYSKLTVDKSRWQQGNVFSCSVMHEAHNHYTQKS
LSLSPGK.

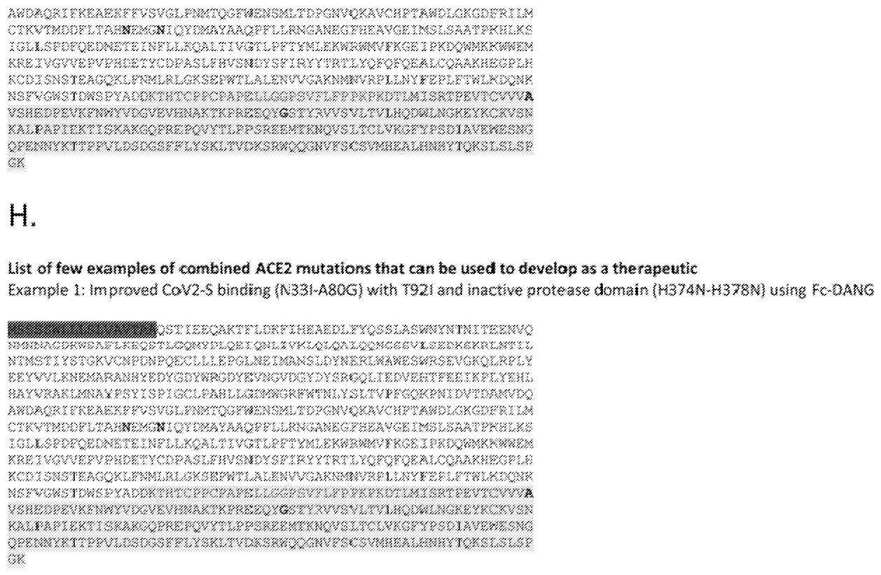

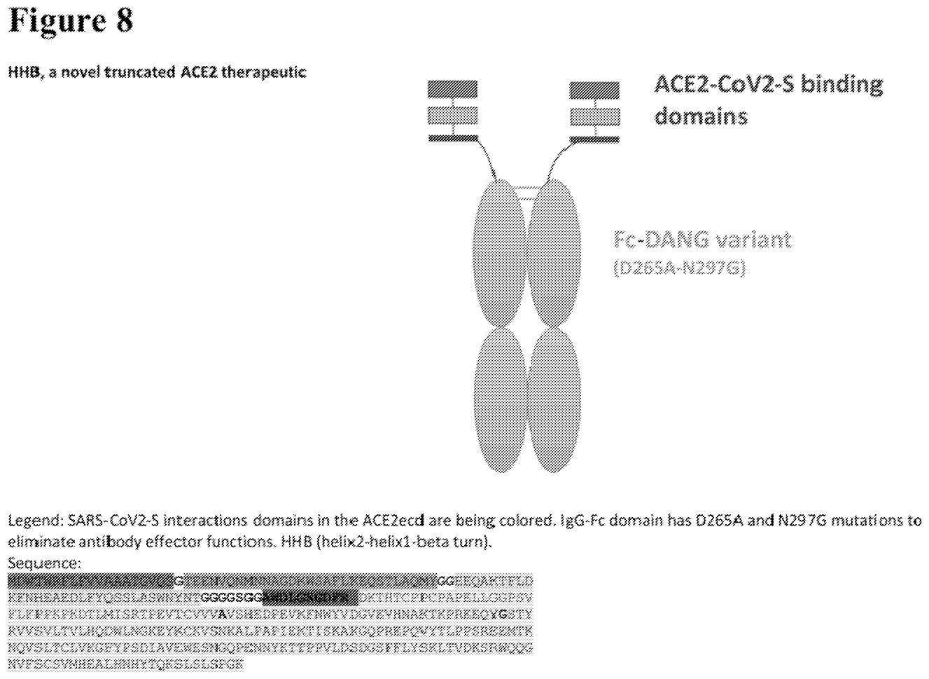

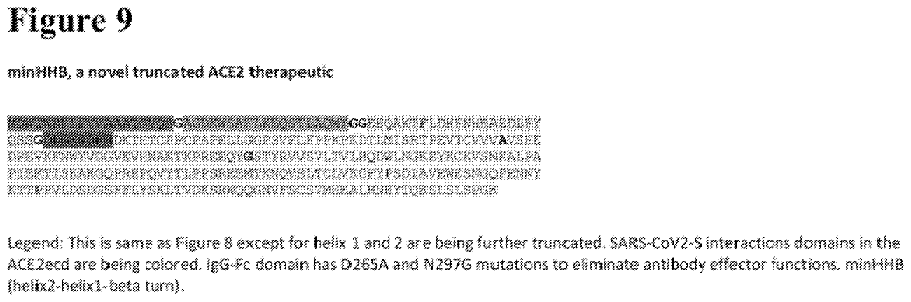

In another embodiment, the protein may include the amino acid sequence as shown in any of FIGS. 8, 9, and 17.

In another embodiment, the HHB-Fc DANG hybrid protein forms a homodimer stabilized by intermolecular disulfide bonds at the hinge region of two polypeptide chains. In a further embodiment, the homodimer is mono-specific but bivalent.

In another embodiment, the protein comprises a synthetic binding domain comprising a combination of segmented ACE2 protein secondary structural motifs, a Fc immunoglobulin fragment and a signal sequence (SS), wherein the segmented ACE2 protein secondary structural motifs are ACE2 helix 2 peptide as provided in SEQ ID NO: 6, ACE2 helix 1 peptide as provided in SEQ ID NO: 7 and ACE2 beta turn peptide as provided in SEQ ID NO: 8, wherein the structural motifs are linked in the order from amino-to-carboxyl direction, ACE2 helix 2 peptide-ACE2 helix 1 peptide-ACE2 beta turn peptide and linked by glycine containing linkers to form helix 2-helix 1-beta turn structure (HHB), wherein the Fc fragment comprises an immunoglobulin heavy chain constant region fragment comprising a hinge region and C_{H}2 and C_{H}3 constant domains, wherein the Fc fragment further comprises D265A and N297G amino acid substitutions reducing or abolishing Fc effector function, and wherein the signal sequence is found at the amino terminus of HHB synthetic binding domain which is linked at its carboxyl terminus to amino terminus of the Fc fragment to form SS-HHB-Fc DANG hybrid protein.

In another embodiment, the SS-HHB-Fc DANG hybrid protein consists of or comprises an amino acid sequence as shown:

(SEQ ID NO: 21)
MDWTWRFLFVVAAATGVQSGTEENVQNMNNAGDRWSAFLKEQSTLAQMYG
GEEQAKTFLDKFNHEAEDLFYQSSLASWNYNTGGGGSGGAWDLGKGDFR
DKTHTCPPCPAPELLGGPSVFLFPPKPKDTLMTSRTPEVTCVVVAVSHED
PEVKFNWYVDGVEVHNAKTKPREEQYGSTYRVVSVLTVLHQDWLNGKEYK
CKVSNKALPAPIEKTISKAKGQPREPQVYTLPPSREEMTKNQVSLTCLVK
GFYPSDIAVEWESNGQPENNYKTTPPVLDSDGSFFLYSKLTVDKSRWQQG
NVFSCSVMHEALHNHYTQKSLSLSPGK.

In another embodiment, the protein may include the amino acid sequence as shown in any of FIGS. 8, 9, and 17.

In another embodiment, the SS-HHB-Fc DANG hybrid protein forms a homodimer stabilized by intermolecular disulfide bonds at the hinge region of two polypeptide chains. In another embodiment, the homodimer is mono-specific but bivalent.

In one embodiment, the protein comprises a synthetic binding domain comprising a combination of segmented ACE2 protein secondary structural motifs and a Fe immunoglobulin fragment, wherein the segmented ACE2 protein secondary structural motifs are minimal ACE2 helix 2 peptide as provided in SEQ ID NO: 9, minimal ACE2 helix 1 peptide as provided in SEQ ID NO: 10 and minimal ACE2 beta turn peptide as provided in SEQ ID NO: 11, wherein the structural motifs are linked in the order from amino-to-carboxyl direction, minimal ACE2 helix 2 peptide-minimal ACE2 helix 1 peptide-minimal ACE2 beta turn peptide and linked by glycine containing linkers to form minimal helix 2-helix 1-beta turn structure (minHHB), wherein the Fc fragment comprises an immunoglobulin heavy chain constant region fragment comprising a hinge region and C_{H}2 and C_{H}3 constant domains, and wherein the minHHB synthetic binding domain is linked to amino terminus of the Fc fragment to form minHHB-Fc hybrid protein. In another embodiment, the minHHB synthetic binding domain binds SARS-CoV-2 virus or SARS-CoV-2 S-protein. In another embodiment, the minHHB-Fc hybrid protein forms a homodimer stabilized by intermolecular disulfide bonds at the hinge region of two polypeptide chains. In yet another embodiment, the homodimer is mono-specific but bivalent.

In one embodiment, the protein comprises a synthetic binding domain comprising a combination of segmented ACE2 protein secondary structural motifs and a Fc immunoglobulin fragment, wherein the segmented ACE2 protein secondary structural motifs are minimal ACE2 helix 2 peptide as provided in SEQ ID NO: 9, minimal ACE2 helix 1 peptide as provided in SEQ ID NO: 10 and minimal ACE2 beta turn peptide as provided in SEQ ID NO: 11, wherein the structural motifs are linked in the order from amino-to-carboxyl direction, minimal ACE2 helix 2 peptide-minimal ACE2 helix 1 peptide-minimal ACE2 beta turn peptide and linked by glycine containing linkers to form minimal helix 2-helix 1-beta turn structure (minHHB), wherein the Fc fragment comprises an immunoglobulin heavy chain constant region fragment comprising a hinge region and C_{H}2 and C_{H}3 constant domains, wherein the Fc fragment further comprises D265A and N297G amino acid substitutions reducing or abolishing Fc effector function, and wherein the minHHB synthetic binding domain is linked to amino terminus of the Fc fragment to form minHHB-Fc DANG hybrid protein. In another embodiment, the minHHB-Fc DANG hybrid protein consists of or comprises an amino acid sequence as shown:

(SEQ ID NO: 22)
GAGDKWSAFLKEQSTLAQMYGGEEQAKTFLDKFNHEAEDLFYQSSGDLGK
GDFRDKTRTCPPCPAPELLGGPSVFLFPPKPKDTLMISRTPEVTCVVVAV
SHEDPEVKFNWYVDGVEVHNAKTKPREEQYGSTYRVVSVLTVLHQDWLNG
KEYKCKVSNKALPAPIEKTTSKAKGQPREPQVYTLPPSREEMTKNQVSLT
CLVKGFYPSDIAVEWESNGQPENNYKTTPPVLDSDGSFFLYSKLTVDKSR
WQQGNVFSCSVMHEALHNHYTQKSLSLSPGK.

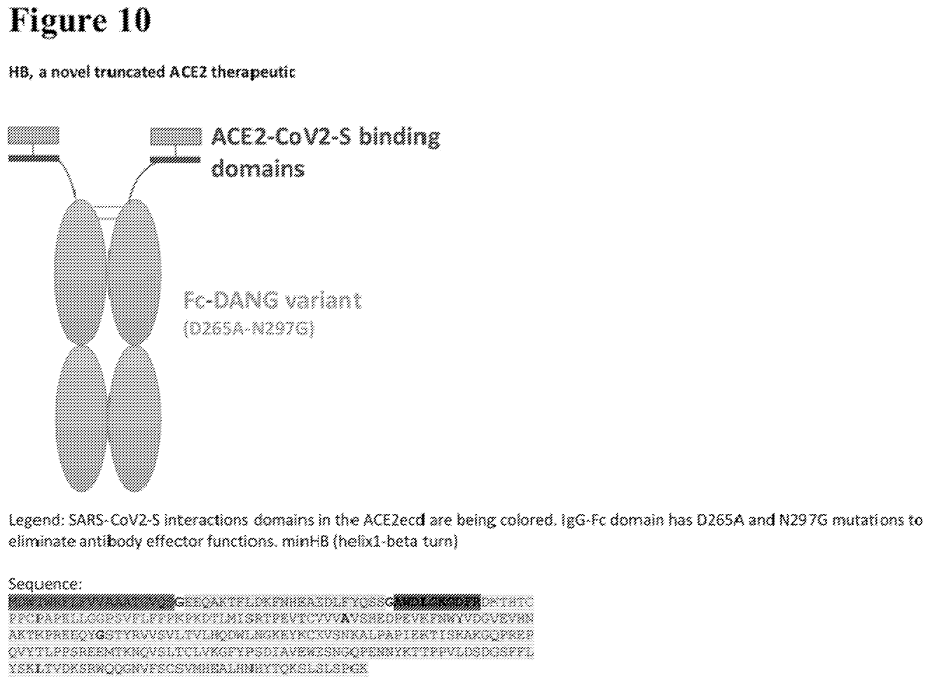

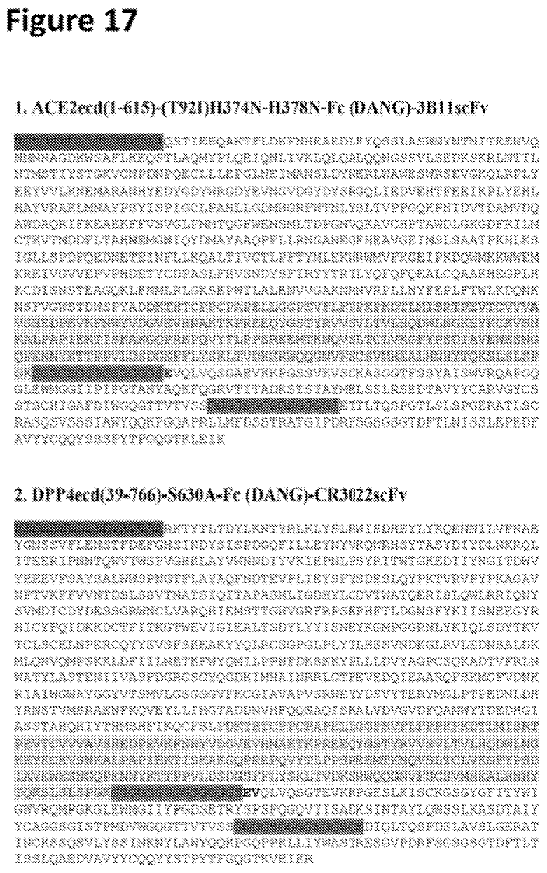

In another embodiment, the protein may include the amino acid sequence as shown in any of FIGS. 10, and 17.

In one embodiment, the minHHB-Fc DANG hybrid protein forms a homodimer stabilized by intermolecular disulfide bonds at the hinge region of two polypeptide chains. In another embodiment, the homodimer is mono-specific but bivalent.

72) In another embodiment, the protein comprises a synthetic binding domain comprising a combination of segmented ACE2 protein secondary structural motifs, a Fe immunoglobulin fragment and a signal sequence (SS), wherein the segmented ACE2 protein secondary structural motifs are minimal ACE2 helix 2 peptide as provided in SEQ ID NO: 9, minimal ACE2 helix 1 peptide as provided in SEQ ID NO: 10 and minimal ACE2 beta turn peptide as provided in SEQ ID NO: 11, wherein the structural motifs are linked in the order from amino-to-carboxyl direction, minimal ACE2 helix 2 peptide-minimal ACE2 helix 1 peptide-minimal ACE2 beta turn peptide and linked by glycine containing linkers to form minimal helix 2-helix 1-beta turn structure (minHHB), wherein the Fc fragment comprises an immunoglobulin heavy chain constant region fragment comprising a hinge region and C_{H}2 and C_{H}3 constant domains, wherein the Fc fragment further comprises D265A and N297G amino acid substitutions reducing or abolishing Fc effector function, and wherein the signal sequence is found at the amino terminus of minHHB synthetic binding domain which is linked at its carboxyl terminus to amino terminus of the Fe fragment to form SS-minHHB-Fc DANG hybrid protein.

In another embodiment, the SS-minHHB-Fc DANG hybrid protein consists of or comprises an amino acid sequence as shown:

(SEQ ID NO: 23)
MDWTWRFLFVVAAATGVQSGACDKWSAFLKEQSTLAQMYGGEEQAKTFLD
KFNHEAEDLFYQSSGDLGKGDFRDKTHTCPPCPAPELLGGPSVFLFPPKP
KDTLMISRTPEVTCVVVAVSHEDPEVKFNWYVDGVEVHNAKTKPREEQYG
STYRVVSVLTVLHQDWLNGKEYKCKVSNKALPAPIEKTISKAKGQPREPQ
VYTLPPSREEMTKNQVSLTCLVKGFYPSDIAVEWESNGQPENNYKTTPPV
LDSDGSFFLYSKLTVDKSRWQQGNVFSCSVMHEALHNAYTQKSLSLSPG
K.

In another embodiment, the protein may include the amino acid sequence as shown in any of FIGS. 10 and 17.

In another embodiment, the protein comprises a synthetic binding domain comprising a combination of segmented ACE2 protein secondary structural motifs and a Fc immunoglobulin fragment, wherein the segmented ACE2 protein secondary structural motifs are minimal ACE2 helix 1 peptide as provided in SEQ ID NO: 10 and ACE2 beta turn peptide as provided in SEQ ID NO: 8, wherein the structural motifs are linked in the order from amino-to-carboxyl direction, minimal ACE2 helix 1 peptide-ACE2 beta turn peptide and linked by glycine containing linkers to form minimal helix 1-beta turn structure (minHB), wherein the Fc fragment comprises an immunoglobulin heavy chain constant region fragment comprising a hinge region and C_{H}2 and C_{H}3 constant domains, and wherein the minHB synthetic binding domain is linked to amino terminus of the Fc fragment to form minHB-Fc hybrid protein. In another embodiment, the minHB synthetic binding domain binds SARS-CoV-2 virus or SARS-CoV-2 S-protein. In another embodiment, the minHB-Fc hybrid protein forms a homodimer stabilized by intermolecular disulfide bonds at the hinge region of two polypeptide chains. In another embodiment, the homodimer is mono-specific but bivalent.

In another embodiment, the protein comprises a synthetic binding domain comprising a combination of segmented ACE2 protein secondary structural motifs and a Fc immunoglobulin fragment, wherein the segmented ACE2 protein secondary structural motifs are minimal ACE2 helix 1 peptide as provided in SEQ ID NO: 10 and ACE2 beta turn peptide as provided in SEQ ID NO: 8, wherein the structural motifs are linked in the order from amino-to-carboxyl direction, minimal ACE2 helix 1 peptide-ACE2 beta turn peptide and linked by glycine containing linkers to form minimal helix 1-beta turn structure (minHB), wherein the Fc fragment comprises an immunoglobulin heavy chain constant region fragment comprising a hinge region and C_{H}2 and C_{H}3 constant domains, wherein the Fc fragment further comprises D265A and N297G amino acid substitutions reducing or abolishing Fc effector function, and wherein the minHB synthetic binding domain is linked to amino terminus of the Fc fragment to form minHB-Fc DANG hybrid protein.

In another embodiment, the minHB-Fc DANG hybrid protein consists of or comprises an amino acid sequence as shown:

(SEQ ID NO: 24)
GEEQAKTFLDKFNHEAEDLEYQSSGAWDLGKGDFRDKTHTCPPCPAPELL
GGPSVFLFPPKPKDTLMISRTPEVTCVVVAVSHEDPEVKFNWYVDGVEVH
NAKTKPREEQYGSTYRVVSVLTVLHQDWLNGKEYKCKVSNKALPAPIEKT
ISKAKGQPREPQVYTLPPSREEMTKNQVSLTCLVKGFYPSDIAVEWESNG
QPENNYKTTPPVLDSDGSFFLYSKLTVDKSRWQQGNVFSCSVMHEALHNH
YTQKSLSLSPGK.

In another embodiment, the protein may include the amino acid sequence as shown in any of FIGS. 10 and 17.

In another embodiment, the minHB-Fc hybrid protein forms a homodimer stabilized by intermolecular disulfide bonds at the hinge region of two polypeptide chains. In another embodiment, the homodimer is mono-specific but bivalent.

In one embodiment, the protein comprises a synthetic binding domain comprising a combination of segmented ACE2 protein secondary structural motifs, a Fc immunoglobulin fragment and a signal sequence (SS), wherein the segmented ACE2 protein secondary structural motifs are minimal ACE2 helix 1 peptide as provided in SEQ ID NO: 10 and ACE2 beta turn peptide as provided in SEQ ID NO: 8, wherein the structural motifs are linked in the order from amino-to-carboxyl direction, minimal ACE2 helix 1 peptide-ACE2 beta turn peptide and linked by glycine containing linkers to form minimal helix 1-beta turn structure (minHB), wherein the Fc fragment comprises an immunoglobulin heavy chain constant region fragment comprising a hinge region and CH2 and CH13 constant domains, wherein the Fc fragment further comprises D265A and N297G amino acid substitutions reducing or abolishing Fc effector function, and wherein the signal sequence is found at the amino terminus of minHB synthetic binding domain which is linked at its carboxyl terminus to amino terminus of the Fc fragment to form SS-minHB-Fc DANG hybrid protein. In another embodiment, the minHB-Fc DANG hybrid protein consists of or comprises an amino acid sequence as shown:

(SEQ ID NO: 25)
MDWTWRFLFVVAAATGVQSGEEQAKTFLDKFNHEAEDLFYQSSGAWDLGR
GDFRDKTHTCPPCPAPELLGGPSVFLFPPKPKDTLMISRTPEVTCVVVAV
SHEDPEVKFNWYVDGVEVHNAKTKPREEQYGSTYRVVSVLTVLHQDWLNG
KEYKCKVSNKALPAPIEKTISKAKGQPREPQVYTLPPSREEMTKNQVSLT
CLVKGFYPSDIAVEWESNGQPENNYKTTPPVLDSDGSFFLYSKLTVDKSR
WQQGNVFSCSVMHEALHNHYTQKSLSLSPGK.

In another embodiment, the protein may include the amino acid sequence as shown in any of FIGS. 10 and 17.

In another embodiment, the minHB-Fc hybrid protein forms a homodimer stabilized by intermolecular disulfide bonds at the hinge region of two polypeptide chains. In another embodiment, the homodimer is mono-specific but bivalent.

80) In one embodiment, the protein comprises a comprises an ACE2 signal sequence and extracellular domain fragment from amino acid residue 1 to 615 of SEQ ID NO: 1 or its variant, a heavy chain constant region fragment corresponding to a Fc portion and a non-ACE2-competing anti-SARS-CoV-2 virus or S-protein diabody or scFv, wherein the ACE2 fragment is linked to amino terminus of the heavy chain constant region fragment, which is in turn linked at its carboxyl terminus to the amino terminus of the diabody or scFv, wherein the ACE2 fragment further comprises H374N and H378N amino acid substitutions, wherein the Fc portion further comprises D265A and N297G amino acid substitutions, and wherein the ACE2 fragment is linked to amino terminus of the heavy chain constant region fragment, which is in turn linked at its carboxyl terminus to the amino terminus of the diabody or scFv to produce a ACE2 extracellular domain fragment-Fc-diabody or scFv fusion protein.

In another embodiment, the protein comprises a homodimer of two identical polypeptides, wherein the polypeptide comprises an ACE2 signal sequence and extracellular domain fragment from amino acid residue 1 to 615 of SEQ ID NO: 1 or its variant, a heavy chain constant region fragment corresponding to a Fc portion and a non-ACE2-competing anti-SARS-CoV-2 virus or S-protein diabody or scFv, wherein the ACE2 fragment is linked to amino terminus of the heavy chain constant region fragment, which is in turn linked at its carboxyl terminus to the amino terminus of the diabody or scFv, wherein the ACE2 fragment further comprises H374N and H378N amino acid substitutions, wherein the Fc portion further comprises D265A and N297G amino acid substitutions, wherein the Fc portion additionally comprises intermolecular disulfide bonds stabilizing the homodimer, and wherein the ACE2 fragment is linked to amino terminus of the heavy chain constant region fragment, which is in turn linked at its carboxyl terminus to the amino terminus of the diabody or scFv to produce a homodimer of ACE2 extracellular domain fragment-Fe-diabody or scFv fusion protein. In another embodiment, the ACE2 extracellular domain fragment-Fe-diabody or scFv fusion protein has reduced or lacks ACE2 peptidase or carboxypeptidase activity. In another embodiment, the ACE2 extracellular domain fragment-Fc-diabody or scFv fusion protein has reduced or lacks Fc effector function. In another embodiment, the variant of ACE2 extracellular domain fragment increases or enhances binding of ACE2 to SARS-CoV-2 virus or SARS-CoV-2 S-protein. In another embodiment, the ACE2 extracellular domain fragment-Fc-diabody or scFv fusion protein is bispecific. In another embodiment, the ACE2 extracellular domain fragment-Fc-diabody or scFv fusion protein is bivalent. In another embodiment, the diabody or scFv is derived from CR3022 scFv or comprises CDRs of CR3022 scFv. In another embodiment, the diabody or scFv and Fc portion is human or humanized.

### Bispecific Antibodies of the Invention

The invention further provides a bispecific knob-hole format ACE2 extracellular domain anti-SARS-Cov-2 S-protein antibody. In one embodiment, the antibody comprises a complex of three polypeptide chains, wherein the first polypeptide comprises a fusion of ACE2 extracellular domain fragment or its variant to amino terminus of an immunoglobulin heavy chain fragment corresponding to Fc portion comprising a hinge region and C_{H}2 and C_{H}3 constant domains, a second polypeptide comprising an immunoglobulin heavy chain comprising a heavy chain variable domain, a hinge region and C_{H}1, C_{H}2 and C_{H}3 constant domains, and a third polypeptide comprising an immunoglobulin light chain comprising a light chain variable domain and a light chain constant region, wherein the C_{H}3 domain of the 1^{st} and 2^{nd} polypeptides are mutated so as to create complementary “knobs” and “holes” based on “knob-in-hole” protein design in order to favor formation of heterodimer between the 1^{st} and 2^{nd} polypeptides, wherein the heterodimer additionally comprises intermolecular disulfide bonds in the hinge region, and wherein the 3^{rd} polypeptide associates with the 2^{nd} polypeptide in order to form an antigen-binding determinant.

In one embodiment, the antigen-binding determinant binds to SARS-CoV-2 virus or SARS-CoV-2 S-protein. In another embodiment, the antigen-binding determinant does not compete with ACE2 binding to SARS-CoV-2 virus or SARS-CoV-2 S-protein. In another embodiment, the antigen-binding determinant is derived from CR3022 scFv or comprises CDRs of CR3022 scFv. In another embodiment, the variable domain of the light chain or heavy chain is derived from CR3022 scFv or comprises one or more CDRs of CR3022 scFv. Examples of the ACE2 extracellular domain fragment include, but are not limited to, a polypeptide from amino acid residue 1-740 of SEQ ID NO: 1, a polypeptide from amino acid residue 1-615 of SEQ ID NO: 1, a polypeptide from amino acid residue 1-393 of SEQ ID NO: 1, a polypeptide with SEQ ID NO: 2, a polypeptide with SEQ ID NO: 3 and a polypeptide with SEQ ID NO: 4.

In one embodiment, the variant of the ACE2 extracellular domain fragment comprises one or more amino acid change in ACE2 fragment which increases binding or binding affinity of the fragment for SARS-CoV-2 virus or SARS-CoV-2 S-protein. In another embodiment, the 1^{st} and 2^{nd} polypeptides additionally comprise D265A and N297G amino acid substitutions in the Fc portion. In another embodiment, the immunoglobulin and ACE2 are human or humanized.

Another embodiments of the invention is an ACE2ecd(1-615)-(T92I)-H374N-H378N-Fc-(DANG)-3B11scFv and DPP4ecd(39-766)-S630A-Fc-(DANG)-CR3022scFv as shown in FIG. 17. These two bi-specific agents can be used to treat three SARS-CoV1, SARS-CoV2, MERS-CoV corona viruses.

### Pharmaceutical Compositions of the Invention

The invention provides a pharmaceutical composition comprising any of the compositions of the invention described herein including isolated SARS-CoV-2 binding protein complexes and bispecific antibodies of the invention above, and one or more pharmaceutically acceptable excipients or carriers.

The invention further provides a pharmaceutical composition comprising the bispecific knob-hole format ACE2 extracellular domain anti-SARS-Cov-2 S-protein antibody of the invention above, and one or more pharmaceutically acceptable excipients or carriers.

In one embodiment, the one or more pharmaceutically acceptable excipients are formulated for delivery as a nasal or oral spray. In another embodiment, the one or more pharmaceutically acceptable excipients or carriers are formulated or carriers are formulated for delivery as a throat lozenge or a cough drop. In another embodiment, the one or more pharmaceutically acceptable excipients or carriers are formulated as a mouth wash. In another embodiment, the one or more pharmaceutically acceptable excipients or carriers are formulated as an injectable drug.

In one embodiment, the one or more pharmaceutically acceptable excipients or carriers are formulated for parenteral administration. Examples of parenteral administration include, but are not limited to, intradermal, subcutaneous, intramuscular, intravenous, intra-arterial, intrathecal, intraperitoneal and intra-articular administration.

In another embodiment, the one or more pharmaceutically acceptable excipients are formulated for oral administration. Examples of forms of oral administration include, but are not limited to, tablet, capsule, soft-gelled capsule, hard-shelled capsule, orally disintegrating tablet, buccal tablet, sublingual table, mini-tablet, effervescent tablet, immediate release tablet, controlled release tablet, immediate-and-controlled release tablet, think film, medicated gum, granule, troche, lozenge, solution, suspension, syrup, emulsion, elixir, and buccal spray.

In another embodiment, the one or more pharmaceutically acceptable excipients are formulated for nasal administration. Examples of forms of nasal administration include, but are not limited to, nasal drop or nasal spray.

In another embodiment, the one or more pharmaceutically acceptable excipients are formulated for inhalation. Examples of forms of inhalation include, but are not limited to, dry powder, lyophilized powder and liquid spray.

In another embodiment, the one or more pharmaceutically acceptable excipients are formulated for ocular administration. Examples of forms of ocular administration include, but are not limited to, solution, emulsion, suspension, ointment, contact lens, implant, insert and intravitreal.

In another embodiment, the one or more pharmaceutically acceptable excipients are formulated for otic administration. Examples of forms of otic administration include, but are not limited to, topical, intratympanic and intracochlear.

In another embodiment, the one or more pharmaceutically acceptable excipients are formulated for topical or transdermal administration. Examples of forms of topical or transdermal administration include, but are not limited to, ointment, cream, lotion, gel, spray and patch.

In another embodiment, the one or more pharmaceutically acceptable excipients are formulated for rectal or vaginal administration. Examples of forms of rectal or vaginal administration include, but are not limited to, suppository, enema, tablet, pessary, gel, cream, foam and sponge

### Nucleic Acids/Vectors/Cells/Host Vector Systems and Methods of Making

The invention further provides a nucleic acid sequence encoding an isolated SARS-CoV-2 binding protein complex of the invention as described herein.

Examples of nucleic acid sequences encoding full length, wild-type human ACE2 protein (SEQ ID NO: 1; UniProtKB ID: Q9BYF1-1) may be accessed under GenBank Accession number: AF291820.1 or AF241254.1. Such coding sequences can be modified to introduce desired mutations as shown in the variants described herein that increases binding or binding affinity for SARS-CoV-2 virus or SARS-CoV-2 S-protein. In addition, the coding sequences provided for full length human ACE2 protein can be truncated using recombinant DNA methods to produce desired ACE2 fragments, so as to practice the full breath of the instant invention. Such fragments may be linked in frame with other coding sequences to produce desired fusion proteins as described herein following introduction to DNA vector, typically providing regulatory signals such as transcriptional promoter/enhancer and terminator, for expression in host systems or in vitro by in vitro transcription-translation system. Further, the nucleic acid sequences which encode amino acid sequences corresponding to polypeptides disclosed in the instant invention can be identified using the GenBank Accession numbers described herein and the gene transcript identifiers. Additionally, based on publicly available codon usage tables, nucleic acid sequence encoding polypeptides of interest can be designed for optimal gene expression for a variety of organisms, including humans (Athey, J. et al. (2017) A new and updated resource for codon usage tables. BMC Bioinformatics. 18 (391): 391; Alexaki, A. et al. (2019) Codon and Codon-Pair Usage Tables (CoCoPUTs): Facilitating Genetic Variation Analyses and Recombinant Gene Design. J. Mol. Biol. 431 (13): 2434-2441).

The invention further provides a nucleic acid encoding a bispecific knob-hole format ACE2 extracellular domain anti-SARS-Cov-2 S-protein antibody of the invention as described herein.

Additionally, the invention provides a vector comprising a nucleic acid of the invention above. The invention also provides a cell comprising a nucleic acid of the invention above. The invention further provides a cell comprising a vector of the invention.

Further, the invention also provides a host vector system, comprising a nucleic acid molecule of the invention above and a host cell. In one embodiment, the host cell is a prokaryote or eukaryote.

The invention also provides methods for making a SARS-CoV-2 binding protein. In one embodiment, the method comprises growing the cells of the invention above under suitable conditions so as to produce the isolated SARS-CoV-2 binding protein.

The invention also provides methods for making a bispecific knob-hole format ACE2 extracellular domain anti-SARS-Cov-2 S-protein antibody. In one embodiment, the method comprises growing the cells of the invention above under suitable conditions so as to produce the isolated SARS-CoV-2 binding protein.

The invention also provides methods for producing a protein comprising growing the host vector systems of the invention in cells above under suitable conditions so as to produce the protein in the host and recovering the protein so produced.

### Formulations and Uses of the Invention

Any of the compositions of the invention described herein including the isolated SARS-CoV-2 complexes, bispecific antibodies and conjugates/fusion proteins containing the ACE2 variants of the invention may be provided in a pharmaceutically acceptable excipient or carrier, and may be in various formulations. As is well known in the art, a pharmaceutically acceptable excipient or carrier is a relatively inert substance that facilitates administration of a pharmacologically effective substance. For example, an excipient can give form or consistency, or act as a diluent. Suitable excipients include but are not limited to stabilizing agents, wetting and emulsifying agents, salts for varying osmolarity, encapsulating agents, buffers, and skin penetration enhancers. Excipients as well as formulations for parenteral and nonparenteral drug delivery are set forth in Remington's Pharmaceutical Sciences 19th Ed. Mack Publishing (1995).

Pharmaceutically acceptable excipients/carriers are generally non-toxic to recipients at the dosages and concentrations employed and are compatible with other ingredients of the formulation. Examples of pharmaceutically acceptable carriers include water, saline, Ringer's solution, dextrose solution, ethanol, polyols, vegetable oils, fats, ethyl oleate, liposomes, waxes polymers, including gel forming and non-gel forming polymers, and suitable mixtures thereof. The carrier may contain minor amounts of additives such as substances that enhance isotonicity and chemical stability. Such materials are non-toxic to recipients at the dosages and concentrations employed, and include buffers such as phosphate, citrate, succinate, acetic acid, and other organic acids or their salts; antioxidants such as ascorbic acid; low molecular weight (less than about ten residues) polypeptides, e.g., polyarginine or tripeptides; proteins, such as serum albumin, gelatin, or immunoglobulin; hydrophilic polymers such as polyvinylpyrrolidone; amino acids, such as glycine, glutamic acid, aspartic acid, or arginine; monosaccharides, disaccharides, and other carbohydrates including cellulose or its derivatives, glucose, mannose, or dextrins; chelating agents such as EDTA; sugar alcohols such as mannitol or sorbitol; counterions such as sodium; and/or nonionic surfactants such as polysorbates, poloxamers, or PEG. Preferably the carrier is a parenteral carrier, more preferably a solution that is isotonic with the blood of the recipient.

Generally, these compositions are formulated for administration by injection or inhalation, e.g., intraperitoneally, intravenously, subcutaneously, intramuscularly, etc. Accordingly, these compositions are preferably combined with pharmaceutically acceptable vehicles such as saline, Ringer's solution, dextrose solution, and the like. The particular dosage regimen, i.e., dose, timing and repetition, will depend on the particular individual and that individual's medical history.

The invention provides a specific formulation comprising an isolated SARS-CoV-2 binding protein complex of the invention mentioned above. In one embodiment, the formulation is a hand or body lotion, cream, emulsion, ointment, gel, spray or patch.

The invention also provides a formulation comprising the bispecific knob-hole format ACE2 extracellular domain anti-SARS-Cov-2 S-protein antibody of the invention mentioned above.

In one embodiment, the formulation may be an eye drop comprising an isolated SARS-CoV-2 binding protein and a stabilizing solution, optionally with a preservative and/or a carrier. In another embodiment, the formulation is a nasal spray or mouth spray. In another embodiment, the formulation is a nasal wash or mouth wash.

### Treatment Methods of the Invention

The invention provides methods for treating a subject infected with SARS-CoV-2 virus with any of the compositions of the invention.

In one embodiment of the invention, the method comprises administering an effective amount of a soluble fragment of angiotensin-converting enzyme 2 (ACE2) so as to inhibit or reduce SARS-CoV-2 virus interaction with ACE2 receptor of the subject so as to limit, inhibit or reduce infection in the subject, thereby treating the subject infected with SARS-CoV-2 virus.

In another embodiment, the method comprises administering an effective amount of an ACE2-Fc fusion protein containing the protease domain 19-617 or deletion of the domain, so as to inhibit or reduce a SARS-CoV-2 virus interaction with ACE2 receptor of the subject so as to limit, inhibit or reduce infection in the subject, thereby treating the subject infected with SARS-CoV-2 virus.

In another embodiment, the method comprises administering an effective amount of an ACE2-Fc with c-terminal anybody fusion (Fab or ScFV) that bind to viral proteins (S-protein, M-protein or N-protein), so as to inhibit or reduce SARS-CoV-2 virus interaction with ACE2 receptor of the subject so as to limit, inhibit or reduce infection in the subject, thereby treating the subject infected with SARS-CoV-2 virus.

The invention also provides methods for inhibiting or reducing SARS-CoV-2 virus infection of a susceptible subject. In one embodiment, the method comprises administering an effective amount of a soluble fragment of angiotensin-converting enzyme 2 (ACE2) so as to inhibit or reduce SARS-CoV-2 virus interaction with ACE2 receptor of the subject, thereby inhibiting or reducing SARS-CoV-2 virus infection of a susceptible subject.

In one embodiment of any of the method above, the amino acid sequence of ACE2 is provided in SEQ ID NO:1 (UniProtKB ID: Q9BYF1-1):

```
                10 20 30 40 50
MSSSSWLLLS LVAVTAAQST IEEQAKTFLD KFNHEAEDLF YQSSLASWNY
                60 70 80 90 100
NTNITEENVQ NMNNAGDKWS AFLKEQSTLA QMYPLQEIQN LTVKLQLQAL
              110 120 130 140 150
QQNGSSVLSE DKSKRLNTIL NTMSTIYSTG KVCNPDNPQE CLLLEPGLNE
              160 170 180 190 200
IMANSLDYNE RLWAWESWRS EVGKQLRPLY EEYVVLKNEM ARANHYEDYG
              210 220 230 240 250
DYWRGDYEVN GVDGYDYSRG QLIEDVEHTF EEIKPLYEHL HAYVRAKLMN
              260 270 280 290 300
AYPSYISPIG CLPAHLLGDM WGRFWTNLYS LTVPFGQKPN IDVTDAMVDQ
              310 320 330 340 350
AWDAQRIFKE AEKFFVSVGL PNMTQGFWEN SMLTDPGNVQ KAVCHPTAWD
              360 370 380 390 400
LGKGDFRILM CTKVTMDDFL TAHHEMGHIQ YDMAYAAQPF LLRNGANEGF
              410 420 430 440 450
HEAVGEIMSL SAATPKHLKS IGLLSPDFQE DNETEINFLL KQALTIVGTL
              460 570 480 490 500
PFTYMLEKWR WMVFKGEIPK DQWMKKWWEM KREIVGVVEP VPHDETYCDP
              510 520 530 540 550
ASLFHVSNDY SFIRYYTRTL YQFQFQEALC QAAKHEGPLH KCDISNSTEA
              560 570 580 590 600
GQKLFNMLPL GKSEPWTLAL ENVVGAKNMN VRPLLNYFEP LFTWLKDQNK
              610 620 630 640 650
NSFVGWSTDW SPYADQSIKV RISLKSALGD KAYEWNDNEM YLFRSSVAYA
              660 670 680 690 700
MRQYFLKVKN QMILFGEEDV RVANLKPRIS FNFFVTAPKN VSDIIPRTEV
              710 720 730 740 750
EKAIRMSRSR INDAFRLNDN SLEFLGIQPT LGPPNQPPVS IWLIVFGVVM
              760 770 780 790 800
GVIVVGIVIL IFTGIRDRKK KNKAPSGENP YASIDISKGE NNPGFQNTDD
VQTSF
```

In one embodiment of any of the method above, the soluble fragment consists of amino acid residues 18-708. In another embodiment of any of the method above, the soluble fragment consists or comprises a protein fragment of at least 35 amino acid residues but less than 805 amino acid residues of ACE2. In yet another embodiment of any of the method above, the soluble fragment consists or comprises a protein fragment of at least 35 amino acid residues but less than 741 amino acid residues of ACE2. In another embodiment of any of the method above, the soluble fragment consists or comprises a protein fragment of at least 35 amino acid residues but less than 617 amino acid residues of ACE2. In another embodiment of any of the method above, the soluble fragment consists or comprises a protein fragment of at least 35 amino acid residues but less than 400 amino acid residues of ACE2. In another embodiment of any of the method above, the soluble fragment consists or comprises a protein fragment of at least 35 amino acid residues but less than 250 amino acid residues of ACE2. In another embodiment of any of the method above, the soluble fragment consists or comprises a protein fragment of at least 35 amino acid residues but less than 150 amino acid residues of ACE2. In another embodiment of any of the method above, the soluble fragment consists or comprises a protein fragment of at least 35 amino acid residues but less than 75 amino acid residues of ACE2. In another embodiment of any of the method above, the soluble fragment consists or comprises a protein fragment of at least 35 amino acid residues but less than 50 amino acid residues of ACE2. In another embodiment of any of the method above, the soluble fragment consists or comprises an ACE2 protein fragment of at least 35 amino acid residues but less than 50 amino acid residues of ACE2.

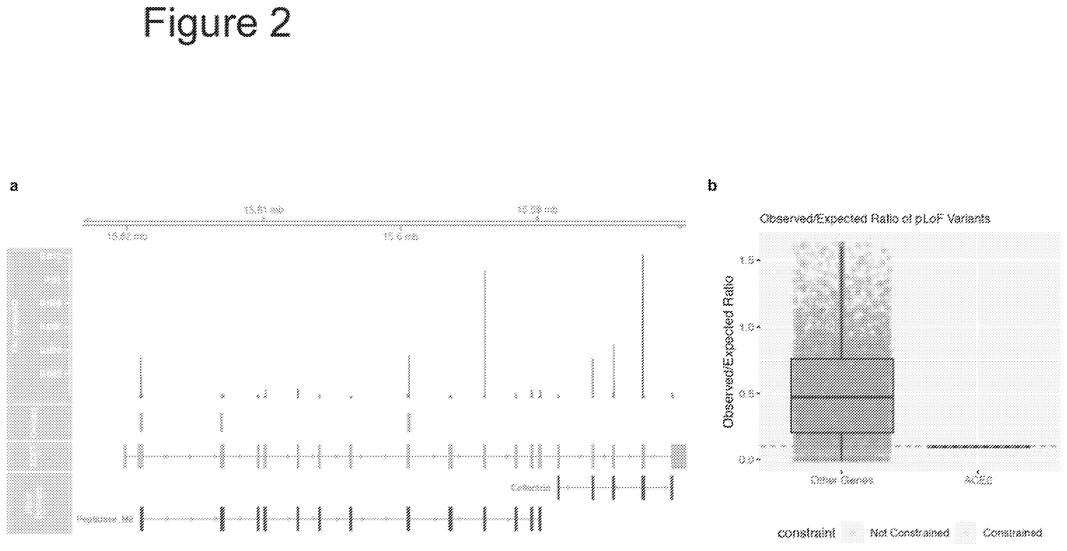

In accordance with the practice of the invention, in one embodiment of any of the method above, the soluble fragment consists or comprises N-terminal domain of ACE2 peptidase domain. In a further embodiment, the peptidase domain consists of amino acid residues 18-606. In another embodiment, the N-terminal domain of ACE2 peptidase domain consists of the SARS-CoV-2 receptor binding site as shown in the SARS-CoV-2 virus RBD footprint of FIG. 2.

In accordance with the practice of the invention, in one embodiment of any of the method above, the soluble fragment has a higher affinity than the same fragment derived from UniProtKB ID: Q9BYF1-1 (SEQ ID NO: 1). In a further embodiment, the soluble fragment having a higher affinity comprises one or more amino acid changes. Examples of the one or more amino acid changes include, but are not limited to, S19P, 121T/V, E23K, A25T, K26E or K26R, T27A, F40L, Q60R, N64K, W69C, T92I, Q102P, Q325R, M366T, D367V, H374R, H378R, M383T, E398D, E398K, T445M, 1446M, and Y510H.

In accordance with the practice of the invention, in one embodiment of any of the method above, the soluble fragment is monomeric. In another embodiment of any of the method above, the soluble fragment is coupled to one or more soluble fragment, so as to produce two or more soluble ACE2 fragments which are linked to each other. In another embodiment of any of the method above, the soluble fragment is coupled to a biologically compatible macromolecule. In another embodiment of any of the method above, the soluble fragment is a chimeric protein. In another embodiment of any of the method above, the soluble fragment is a recombinant protein.

In accordance with the practice of the invention, in one embodiment of any of the method above, the subject is a mammal. In a further embodiment, the mammal is a human. Examples of mammals include, but are not limited to, a human or an animal such as a non-human primate, pig, mouse, rat, dog, cat, horse, monkey, ape, rabbit or cow.

### Monitoring Methods of the Invention

The invention also provides methods for monitoring the course of a SARS-CoV-2 infection in a subject using any of the compositions of the invention. In one embodiment, the method comprises obtaining a sample from the subject, determining amino acid sequence of ACE2 of the subject, comparing identity of amino acid so determined to reference amino acids known to affect SARS-CoV-2 interaction with ACE2, wherein finding an amino acid change favoring interaction with surface spike glycoprotein, S protein, of SARS-CoV-2 are any of S19P, I21T/V, E23K, A25T, K26E or K26R, T27A, F40L, Q60R, N64K, W69C, T92I, Q102P, Q325R, M366T, D367V, H374R, H378R, M383T, E398D, E398K, T445M, I446M, and Y510H, and wherein an amino acid change resulting in less favorable interaction with S protein of SARS-CoV-2 are any of K31R, N33I, H34R, E35K, E37K, D38V, Y50F, N51D or N51S, M62I or M62V, A65S, K68E, F72H, M82I, Y83H, P84T, V93G, N290H, G326E, E329G, P346S, G352V, D355N, T371K, Q388L, P389H, F504I or F504L, H505R, D509Y, S511P, R514G, Y515C and R518T and predicting a subject to have a more severe course of infection for the subject with an amino acid change favoring interaction with S protein of SARS-CoV-2 or a milder course of infection for the subject with an amino acid change resulting in less favorable interaction with S protein of SARS-CoV-2.

The invention additionally provides methods for assessing risk of being infected by SARS-CoV-2 virus in a subject using any of the compositions of the invention. In one embodiment, the method comprises obtaining a sample from the subject, determining amino acid sequence of ACE2 of the subject, comparing identity of amino acid so determined to reference amino acids known to affect SARS-CoV-2 interaction with ACE2, wherein finding an amino acid change resulting in increased risk of being infected are any of S19P, I21T/V, E23K, A25T, K26E or K26R, T27A, F40L, N64K, Q60R, N64K, W69C, T92I, Q102P, Q325R, M366T, D367V, H374R, H378R, M383T, E398D, E398K, T445M, I446M, and Y510H, and wherein an amino acid change resulting in decreased risk of being infect are any of K31R, N33I, H34R, E35K, E37K, D38V, Y50F, N51D or N51S, M62I or M62V, A65S, K68E, F72H, M82I, Y83H, P84T, V93G, N290H, G326E, E329G, P346S, G352V, D355N, T371K, Q388L, P389H, F504I or F504L, H505R, D509Y, S5M1P, R514G, Y515C and R518T and predicting a subject to have an increased or decreased risk based on finding a match falling into the two groups.

### Detection Methods of the Invention

The invention further provides methods for determining presence of SARS-CoV-2 virus or SARS-CoV-2 S-protein in a sample using any of the compositions of the invention. In one embodiment, the method comprises applying a fixed volume of a sample to the lateral flow diagnostic cassette of the invention mentioned above. In another embodiment, the method further comprises adding a fixed volume of the buffer. In another embodiment, the method further comprises waiting for a prescribed amount of time. In another embodiment, the method further comprises examining the cassette for emergence of visible lines. In another embodiment, the method further comprises determining the number and location of one or more lines; wherein presence of one line further away from the sample well indicates absence of or below detection limit for SARS-CoV-2 virus or SARS-CoV-2 S-protein, presence of two lines each line closest to edge of window of the cassette indicate presence of SARS-CoV-2 virus or SARS-CoV-2 S-protein, and presence of three lines or no line indicates a lack of confidence in the test result, thereby determining presence of SARS-CoV-2 virus or SARS-CoV-2 S-protein in a sample.

In one embodiment, the sample is a liquid or liquid-air mixture. Examples of the liquid or liquid-air mixture include, but are not limited to, blood, serum, bodily fluid, saliva, nasal drip, respiratory droplet, aerosol, sputum, phlegm, mucus, secretion, urine, fecal material, tissue culture media, spent media, biological extract, known SARS-CoV-2-containing fluid, and suspect SARS-CoV-2 containing fluid. In a preferred embodiment, the sample is human blood, serum, or a bodily fluid.

In another embodiment of the method for determining presence of SARS-CoV-2 virus or SARS-CoV-2 S-protein in a subject, the method comprises attaching a nose cone of the lateral flow diagnostic kit of the invention for directing nasal spray to the sample well or a mask of the invention.

In another embodiment, the method for determining presence of SARS-CoV-2 virus or SARS-CoV-2 S-protein in a subject further comprises placing the sample well of the lateral flow diagnostic cassette of the lateral flow diagnostic kit of the invention directly under the second opening. In another embodiment, the method further comprises forcefully expelling air through a nostril attached to the nose cone or coughing through the mouth covered with the mask. In another embodiment, the method further comprises repeating the expelling step mentioned above if required or desired. In another embodiment, the method further comprises adding a fixed volume of the buffer of the invention mentioned above. In another embodiment, the method further comprises waiting for a prescribed amount of time. In another embodiment, the method further comprises examining the cassette for emergence of visible lines. In another embodiment, the method further comprises determining the number and location of one or more lines; wherein presence of one line further away from the sample well indicates absence of or below detection limit for SARS-CoV-2 virus or SARS-CoV-2 S-protein, presence of two lines each line closest to edge of window of the cassette indicate presence of SARS-CoV-2 virus or SARS-CoV-2 S-protein, and presence of three lines or no line indicates a lack of confidence in the test result, thereby determining presence of SARS-CoV-2 virus or SARS-CoV-2 S-protein in a sample.

In another embodiment of the method, the method comprises immobilizing the isolated SARS-CoV-2 binding protein complex of the invention mentioned above or a fragment thereof lacking a signal sequence on a surface of a solid support. In another embodiment, the method further comprises contacting the isolated SARS-CoV-2 binding protein of the immobilization step above with the sample. In another embodiment, the method further comprises washing unbound sample off the immobilizing surface. In another embodiment, the method further comprises contacting the immobilizing surface with a biotinylated CR3022 antibody in a full-length immunoglobulin format wherein biotin is conjugated to Fc portion of the immunoglobulin. In another embodiment, the method further comprises washing unbound biotinylated CR3022 antibody off the immobilizing surface. In another embodiment, the method further comprises contacting the immobilizing surface with streptavidin conjugate horse radish peroxidase. In another embodiment, the method further comprises washing unbound streptavidin conjugate horse radish peroxidase off the immobilizing surface. In another embodiment, the method further comprises contacting the immobilizing surface with a chromogenic or fluorogenic substrate for horse radish peroxidase for a fixed length of time. In another embodiment, the method further comprises determining presence of a colored or fluorescent product; wherein presence of a colored or fluorescent product above negative control background indicates presence of SARS-CoV-2 virus or SARS-CoV-2 S-protein in the sample.

The invention further provides methods for quantifying amount of SARS-CoV-2 virus or SARS-CoV-2 S-protein in a sample. In one embodiment, the method comprises immobilizing the isolated SARS-CoV-2 binding protein complex of the invention mentioned above or a fragment thereof lacking a signal sequence on a surface of a solid support. In another embodiment, the method further comprises contacting the isolated SARS-CoV-2 binding protein of the immobilization step above with the sample or a reference SARS-CoV-2 virus or SARS-CoV-2 S-protein serially diluted. In another embodiment, the method further comprises washing unbound sample off the immobilizing surface. In another embodiment, the method further comprises contacting the immobilizing surface with a biotinylated CR3022 antibody in a full-length immunoglobulin format wherein biotin is conjugated to Fc portion of the immunoglobulin. In another embodiment, the method further comprises washing unbound biotinylated CR3022 antibody off the immobilizing surface. In another embodiment, the method further comprises contacting the immobilizing surface with streptavidin conjugate horse radish peroxidase. In another embodiment, the method further comprises washing unbound streptavidin conjugate horse radish peroxidase off the immobilizing surface. In another embodiment, the method further comprises contacting the immobilizing surface with a chromogenic or fluorogenic substrate for horse radish peroxidase for a fixed length of time. In another embodiment, the method further comprises detecting and quantifying amount of colored or fluorescent product produced by the sample and the serially diluted reference. In another embodiment, the method further comprises estimating the amount of SARS-CoV-2 virus or SARS-CoV-2 S-protein in the sample by comparing amount of colored or fluorescent product for the sample with that quantified for the serially diluted reference, thereby, quantifying the amount of SARS-CoV-2 virus or SARS-CoV-2 S-protein in a sample.

In one embodiment of the method, the sample is human blood, serum, or a bodily fluid. In another embodiment, the sample is a liquid or liquid-air mixture. Examples of the liquid or liquid-air mixture include, but are not limited to, blood, serum, bodily fluid, saliva, nasal drip, respiratory droplet, aerosol, sputum, phlegm, mucus, secretion, urine, fecal material, tissue culture media, spent media, biological extract, known SARS-CoV-2-containing fluid, and suspect SARS-CoV-2 containing fluid.

### Kits of the Invention

The present invention provides kits (i.e., a packaged combination of reagents with instructions) containing the active agents of the invention (i.e., any of the compositions of the invention described herein) useful for detecting, diagnosing, monitoring or treating COVID-19 diseases and/or conditions.

The kit can contain a pharmaceutical composition that includes one or more agents of the invention effective for detecting, diagnosing, monitoring or treating COVID-19 and an acceptable carrier or adjuvant, e.g., pharmaceutically acceptable buffer, such as phosphate-buffered saline, Ringer's solution or dextrose solution. It may further include other materials desirable from a commercial and user standpoint, including other buffers, diluents, filters, needles, syringes, and package inserts with instructions for use.

The agents may be provided as dry powders, usually lyophilized, including excipients that upon dissolving will provide a reagent solution having the appropriate concentration.

The kit comprises one or more containers with a label and/or instructions. The label can provide directions for carrying out the preparation of the agents for example, dissolving of the dry powders, and/or detecting, diagnosing, monitoring or treating COVID-19.

The label and/or the instructions can indicate directions for in vivo use of the pharmaceutical composition. The label and/or the instructions can indicate that the pharmaceutical composition is used alone, or in combination with another agent to detecting, diagnosing, monitoring or treating COVID-19.

The label can indicate appropriate dosages for the agents of the invention as described supra.

Suitable containers include, for example, bottles, vials, and test tubes. The containers can be formed from a variety of materials such as glass or plastic. The container can have a sterile access port (for example the container can be an intravenous solution bag or a vial having a stopper pierceable by a needle such as a hypodermic injection needle).

The invention further provides a lateral flow diagnostic kit for detection of SARS-CoV-2 virus or SARS-CoV-2 S-protein in a sample. In one embodiment, the kit comprises: a cassette comprising a sample well and one or more windows encasing a solid support for one or more capillary beds arranged in the order of: i) a first sample pad for absorption of sample, initiating capillary action and directly forming floor of the sample well; ii) a second conjugation pad comprising a mixture of gold-labelled SARS-CoV-2 binding protein comprising a human ACE2 extracellular domain fragment and a human Fc fragment and a gold-labelled rabbit IgG positive control antibody for interrogating the sample; iii) a third membrane pad visible through one or more windows for inspecting test lines, wherein the membrane pad comprises three separate lines of immobilized antibodies in the order from closest to furthest from the sample well: immobilized CR3022 antibody for binding SARS-CoV-2 virus or SARS-CoV-2 S-protein, IgG1 antibody for negative control, and anti-rabbit IgG for positive control; iv) a fourth absorption pad to wick excess fluid. The kit further comprises a buffer for maintaining capillary action to be applied after the sample to the sample well, and instruction for use.

In another embodiment of the kit, the isolated SARS-CoV-2 binding protein is that of the protein of the invention mentioned above. In another embodiment, the CR3022 antibody is an scFv, an immunoglobulin or an immunoglobulin fragment comprising CDRs of CR3022. In another embodiment, the sample is a liquid or liquid-air mixture. Examples of the liquid or liquid-air mixture include, but are not limited to, blood, serum, saliva, nasal drip, respiratory droplet, aerosol, sputum, secretion, urine, fecal material, bodily fluid, tissue culture media, spent media, biological extract, known SARS-CoV-2-containing fluid, and suspect SARS-CoV-2 containing fluid.

In another embodiment, the kit further comprises a nose cone for directing nasal spray to the sample well. In another embodiment, the nose cone comprises one opening that fits into one nostril, or over at least one nostril, and a second opening to place over the sample well, and a channel between the two openings so as to direct air forcedly expelled through a nostril of the subject to the sample well. In another embodiment, the nose cone comprises a porous or non-porous material. In another embodiment, the nose cone comprises a contiguous channel wall or a channel wall designed to release air. In another embodiment, the nose cone fit tightly or snuggly at both openings the channel comprises a semi-porous material or a vent to release air. In another embodiment, the kit further comprises a mask for directing a cough to the sample well. In another embodiment, the mask comprises one opening that fits tightly or snuggly on a face covering the mouth, and a second opening to place over the sample well, and a channel between the two openings so as to direct air forcedly expelled through the mouth of the subject to the sample well. In a further embodiment, the mask comprises a porous or non-porous material. In a further embodiment, the mask comprises a contiguous channel wall or a channel wall designed to release air. In another embodiment, the mask fits tightly at both openings the channel comprises a hole sufficient to release air or a vent to release air. In a further embodiment, the sample is a liquid or liquid-air mixture. Examples of the liquid or liquid-air mixture include, but are not limited to, blood, serum, bodily fluid, saliva, nasal drip, respiratory droplet, aerosol, sputum, phlegm, mucus, secretion, urine, fecal material, tissue culture media, spent media, biological extract, known SARS-CoV-2-containing fluid, and suspect SARS-CoV-2 containing fluid. In a preferred embodiment, the sample is human blood, serum, or a bodily fluid.

The invention also provides kits comprising the isolated SARS-CoV-2 binding protein complex of the invention above and a label or instructions for use.

Additionally, the invention provides kits comprising the bispecific knob-hole format ACE2 extracellular domain anti-SARS-Cov-2 S-protein antibody of the invention and a label or instruction for use.

Additionally, the invention provides the nucleic acid of the invention above and a label or instruction for use.

Additionally, the invention provides kits comprising the vector of the invention above and a label or instruction for use.

Additionally, the invention provides kits comprising the cell of the invention above and a label or instruction for use.

In a further embodiment, the present invention provides kits (i.e., a packaged combination of reagents with instructions) containing the active agents of the invention useful for assessing risk or course of a SARS-CoV-2 infection such as oligonucleotide or nucleic acid fragment for assessing polymorphism of ACE2 gene.

The kit can contain a pharmaceutical composition that includes one or more agents of the invention (such as oligonucleotide or nucleic acid fragment for assessing polymorphism of ACE2 gene) effective for treating or assessing risk or course of a SARS-CoV-2 infection and an acceptable carrier or adjuvant, e.g., pharmaceutically acceptable buffer, such as phosphate-buffered saline, Ringer's solution or dextrose solution. It may further include other materials desirable from a commercial and user standpoint, including other buffers, diluents, filters, needles, syringes, and package inserts with instructions for use.

The agents may be provided as dry powders, usually lyophilized, including excipients that upon dissolving will provide a reagent solution having the appropriate concentration.

The kit may comprise one or more containers with a label and/or instructions. The label can provide directions for carrying out the preparation of the agents for example, dissolving of the dry powders, and/or treatment or assessing risk or course of a SARS-CoV-2 infection.

The label and/or the instructions can indicate directions for in vivo use of the pharmaceutical composition. The label and/or the instructions can indicate that the pharmaceutical composition is used alone, or in combination with another agent to treat or assess risk or course of a SARS-CoV-2 infection.

The label can indicate appropriate dosages for the agents of the invention as described supra.

Suitable containers include, for example, bottles, vials, and test tubes. The containers can be formed from a variety of materials such as glass or plastic. The container can have a sterile access port (for example the container can be an intravenous solution bag or a vial having a stopper pierceable by a needle such as a hypodermic injection needle).

According to another aspect of the invention, kits for assessing risk or course of a SARS-CoV-2 are provided. In one embodiment, the kit comprises oligonucleotide or nucleic acid fragment for assessing polymorphism of ACE2 gene and instruction for use. In a further embodiment, the polymorphism is directed to the coding region of the ACE2 gene. In another embodiment, the polymorphism is directed to the SARS-CoV-2 S protein interaction site on ACE2 protein as provided in FIG. 2. In an additional embodiment, the oligonucleotide or nucleic acid fragment is used to assess the status of the first 115 codons of ACE2 gene.

According to another aspect of the invention, kits for detecting SARS-CoV-2 comprising an ACE2 variant from any of the Tables herein and an informational insert are also provided.

The invention also provides a filter, membrane, fabric, polyester, cloth, cotton, mask, screen, fiber, carbon fiber, granule, nanoparticle, gold particle, nanotube, computer chip, surface plasmon resonance (SPR) chip, biosensor chip, glass, plastic, non-porous material or porous material coated, modified or impregnated with The isolated SARS-CoV-2 binding protein complex of the invention mentioned above, so as to trap or capture SARS-CoV-2 virus or SARS-CoV-2 S-protein.

Additionally, the invention provides a filter, membrane, fabric, polyester, cloth, cotton, mask, screen, fiber, carbon fiber, granule, nanoparticle, gold particle, nanotube, computer chip, surface plasmon resonance (SPR) chip, biosensor chip, glass, plastic, non-porous material or porous material coated, modified or impregnated with the bispecific knob-hole format ACE2 extracellular domain anti-SARS-Cov-2 S-protein antibody of the invention mentioned above, so as to trap or capture SARS-CoV-2 virus or SARS-CoV-2 S-protein.

It is to be understood that this invention is not limited to the particular methodology, protocols, cell lines, animal species or genera, constructs, and reagents described, as such may, of course, vary. It is also to be understood that the terminology used herein is for the purpose of describing particular embodiments only, and is not intended to limit the scope of the present invention, which will be limited only by the appended claims.

Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, the preferred methods, devices and materials are now described.

All publications mentioned herein are incorporated herein by reference for the purpose of describing and disclosing, for example, the reagents, cells, constructs, and methodologies that are described in the publications, and which might be used in connection with the presently described invention. The publications discussed above and throughout the text are provided solely for their disclosure prior to the filing date of the present application. Nothing herein is to be construed as an admission that the inventors are not entitled to antedate such disclosure by virtue of prior invention.

The following examples are put forth so as to provide those of ordinary skill in the art with a complete disclosure and description of how to make and use the subject invention, and are not intended to limit the scope of what is regarded as the invention. Efforts have been made to ensure accuracy with respect to the numbers used (e.g. amounts, temperature, concentrations, etc.) but some experimental errors and deviations should be allowed for. Unless otherwise indicated, parts are parts by weight, molecular weight is average molecular weight, temperature is in degrees centigrade; and pressure is at or near atmospheric.

### EXAMPLES

### Example 1

### Methods

### Identification of ACE2 Polymorphisms

We queried multiple genomic databases including gnomaAD (Karczewski et al., 2019) (https://gnomad.broadinstitute.org/), DicoverEHR (Dewey et al., 2016), RotterdamStudy (Ikram et al., 2017), ALSPAC (Fraser et al., 2013) and Asian specific databases which included GenomeAsia100k (GenomeAsia, 2019), HGDP (Bergstrom et al., 2020), TOMMO-3.5kjpnv2 (Tadaka et al., 2019) IndiGen (https://indigen.igib.in/) and other aggregated data for ACE2 protein altering variations in populations groups across the world. The ACE2 genotypes in this study were from over 290,000 samples representing over 400 population groups across the world.

### Fst Analysis

To assess genetic variation in the coding region of ACE2, we calculated the fixation index (Fst) from 2,381 unrelated individuals across 26 populations in the 1000 Genomes Project Phase 3 and 57,783 female individuals across eight populations in gnomAD. For 1000 Genome data, we used the Weir and Cockerham (1984) method as implemented in vcftools (Version 0.1.17); the weighted Fst were calculated from 88 variants. For gnomAD (v2.1.1), because we only have access to the allele counts, we used the original formulation by Wright (1969) and reported the weighted mean Fst as described in Bhatia et al. (2013); 277 variants were used. Because Fst values vary based on variants used (Bhatia et al. 2013), we calculated the Fst in a set of randomly selected genes on the same chromosomes matched by the length decile to use for comparison. To assess if variants in the peptidase domain has lower genetic variation, we used the one-sided Wilcoxon rank-sum test to compare 15 variants in the peptidase domain against 50 variants outside. Variants with Fst<1e-4 were removed as they were uninformative.

### Genealogical Estimation of Variant Age (GEVA)

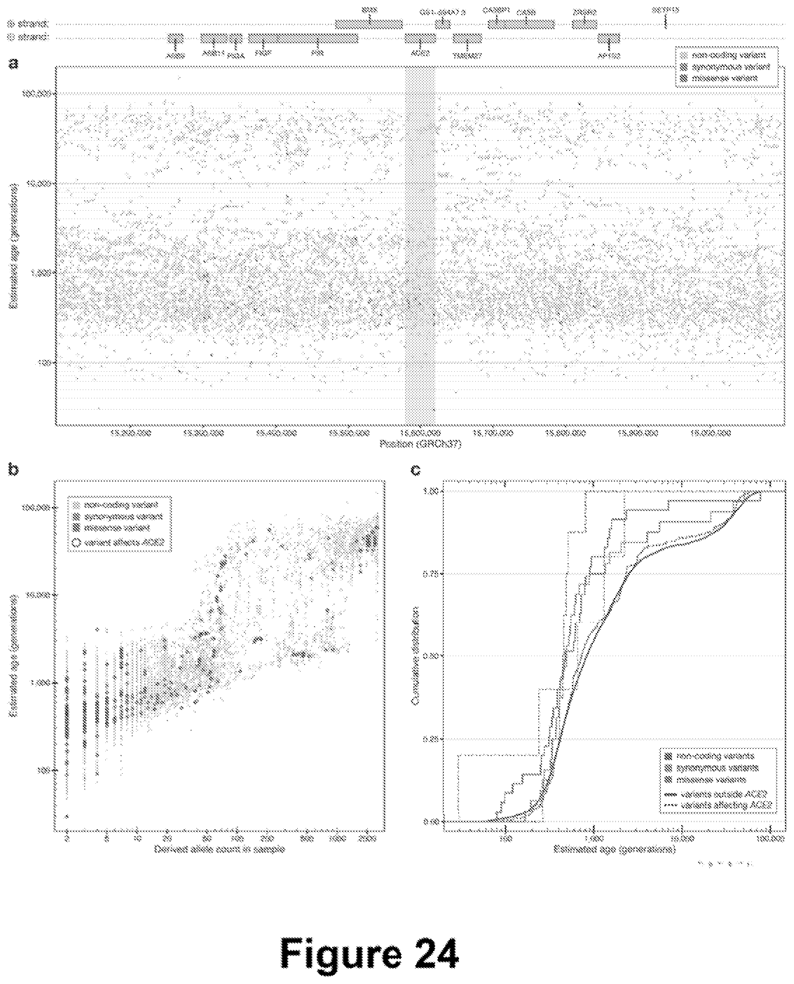

We used data from the 1000 Genomes Project (Genomes Project et al., 2015) to estimate the time of mutation of all variants located within a 1 Mb region around the ACE2 gene on Chromosome X, from the female-only subset of 1,271 individuals (FIG. 24a). As previously described (Albers and McVean, 2020), we performed the analysis using an effective population size of Ne=10,000, mutation rate μ=1.2×10-8, and with variable recombination rates according to HapMap2 (International HapMap et al., 2007). We used the most recent version of GEVA software (https://github.com/pkalbers/geva/tree/aneallele), which allowed us to provide external information about predicted ancestral and derived allelic states from Ensembl (release 95) to correct model assumptions for all variants on Chromosome X. Variant age is estimated through pairwise analyses between haplotype sequences which may or may not carry the derived allele at a given variant. We analyzed each variant using a maximum of 5,000 concordant pairs (carrier and carrier haplotypes) and 5,000 discordant pairs (carrier and non-carrier) to achieve high confidence. We further distinguished variants into non-coding, synonymous, and missense variants using the Ensembl Variant Effect predictor (release 95) (McLaren et al., 2016) and separated variants affecting ACE2 (n=385) from those outside the ACE2 gene region (n=9,095). The proportion of rare variants (≤0.1% frequency of the derived allele within the sample) was similar in both groups; 19% and 22%, respectively (FIG. 24b). For variants outside the ACE2 gene, we found that 54% of non-coding variants were estimated to have arisen within the last 1,000 generations, compared to 75% of synonymous and 80% of missense variants (FIG. 24c). This suggests that past selective pressure may have acted more strongly to prune mutations that occur within the coding region of the genome. We found that this signal was more pronounced for missense mutations affecting ACE2, where we found 58% of non-coding and 60% of synonymous variants to be younger than 1,000 generations, but where all missense variants were younger than approximately 800 generations. The average age (±SE) of missense variants affecting ACE2 was 472 (±58) generations, compared to 3,016 (+2198) generations for variants outside the ACE2 gene region. However, the low number of coding variants found within the focal 1 MB region for which we were able to estimate the age (n=43 missense and n=37 synonymous variants) makes such comparisons difficult.

### ACE2 Ortholog Sequence Analysis

A total of 295 Human ACE2 orthologs were obtained from NCBI (Table 2 for accession numbers). A snake ACE2 ortholog protein was obtained from the published Indian cobra genome (Suryamohan et al., 2020). Multiple sequence alignment of residues surrounding the ACE2 NxT/S motif was performed using MCoffee (www.tcoffee.org). Phylogenetic trees were constructed using the PhyML webserver (www.phylogeny.fr).

### Structural Analysis

Each identified variant was mapped, modeled, and analyzed in Pymol using the recently deposited crystal structures 6VW1 and 6LZG of human ACE2 bound to either chimeric SARS CoV-2 RBD (6VW1) or complete SARS CoV-2 RBD (6LZG).

### Cloning and Protein Expression

Extracellular domain (amino acids 1-615; NP_001358344) of human ACE2 (hACE2) WT or variants with a c-terminal 8×-His or human-Fc tag was synthesized (IDT, USA) and cloned into a CMV promoter driven mammalian expression vector. Human codon optimized CoV-2-S-RBD (amino acids 319-541; YP_009724390) sequence with a c terminal 8×His-tag were synthesized and cloned into a CMV promoter driven mammalian expression vector. The prefusion SARS-CoV-2 S-protein trimer stabilized ectodomain (amino acids 1-1208; YP_009724390), as previously described (Wrapp et al., 2020), containing K986P, V987P, RRAR to GSAS (residues 682-685) at the furin cleavage site, a C-terminal T4 fibritin trimerization motif, an HRV3C protease cleavage site, a TwinStrep-tag and a 8×Hi-tag was synthesized and expressed using a CMV promoter. Sequence verified plasmids prepared using NucleoBond® Xtra Midi kit (Takara Bio USA, Inc) were transfected into 293 cells using FectoPro (Polyplus, USA). Proteins were purified from media 3-5 days post transfection using Protein A GraviTrap column or His GraviTrap column (GE Healthcare).

### ELISA Affinity Studies

The affinity of S-RBD or S1 for hACE2-Fc WT or variants was measured using a standard ELISA assay. Briefly, purified CoV2-S-RBD (2 μg/mL) or S1 (2 μg/mL) or the prefusion S-protein trimer (2 μg/mL) was coated onto 96-well ELISA plates (Fisher Scientific, #07-000-102) and incubated at 4° C. for 18 h. The coated plates were washed three times with 200 μl of PBST and then blocked with 200 ul of 3% BSA (Sigma-Aldrich #A8327) in PBST (Sigma-Millipore #524653) and incubated for 1h at room temperature. After washing the plates three times with 200 μl of PBST an increasing concentration of hACE2-Fc proteins were added and incubated for 1 h at room temperature. The unbound hACE2-Fc was removed by washing the plate three times with 200 μl of PBST. The bound hACE2 was detected using Goat-anti-human-IgG-Fc HRP (Jackson Immuno Research #109-035-008; 1:5000 dilution) using 50 μl TMB substrate (Pierce/Thermo Fisher Scientific #34028). After 3 minutes, the reaction was stopped using 50 μL of 2N H2SO4. The optical density of the reaction was measured at 450 nm using a plate reader (Molecular Devices Gemini XPS). The data was analyzed and EC50 was calculate using Prism (GraphPad).

### Example 2

### Human ACE2 Population Polymorphism

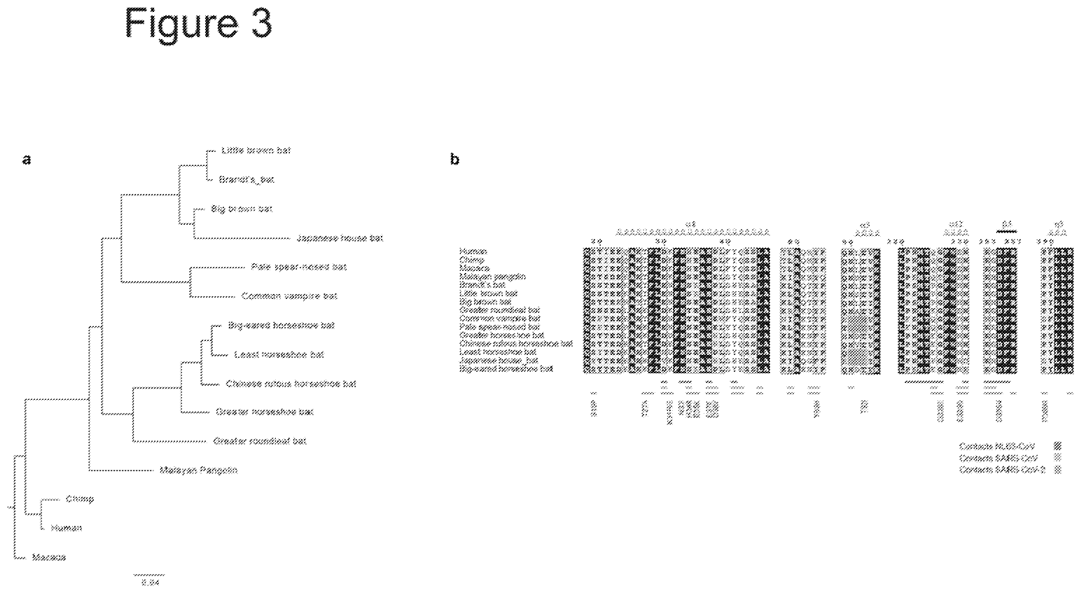

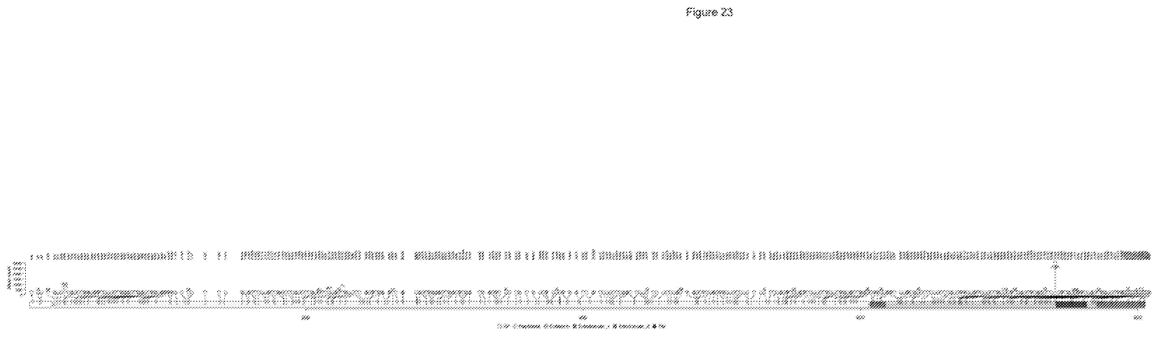

The SARS-CoV-2 S-protein interacts with the ACE2 PD to enter the human host cells. Analysis of the RBD domain of SARS-CoV-2, SARS-CoV and bat CoV RaTG13 S-proteins identified changes that have increased the affinity of CoV-2 S1 RBD to human ACE2, which likely contributes to its increased infectivity (Shang et al., 2020; Wrapp et al., 2020). It is very likely that there exists ACE2 variants in human populations, though not under selection, that may increase or decrease its affinity to SARS-CoV-2 S-protein and thereby render individuals more resistant or susceptible to the virus. To investigate this, we assessed ACE2 protein-altering variations from a number of databases including gnomAD (Karczewski et al., 2019), RotterdamStudy (Ikram et al., 2017), ALSPAC (Fraser et al., 2013) and Asian-specific databases which included GenomeAsia100k (GenomeAsia, 2019), TOMMO-3.5kjpnv2 (Tadaka et al., 2019), and IndiGen (https://indigen.igib.in/), and HGDP (Bergstrom et al., 2020) (Table 1). We found a total of 298 unique protein altering variants across 256 codons distributed throughout the 805 amino acid long human ACE2 (FIGS. 1a and 1c; FIG. 23 and Table 1). The most frequent variant, N720D (1.6% allele frequency; n=3054, gnomAD), was found in the C-terminal collectrin domain that is not involved in the SARS-CoV-2 S-protein interaction. Overall, we found human ACE2 receptor polymorphisms to be low with a weighted mean Fst (fixation index) value of 0.0168, and the ACE2 PD showed even more reduced variation (Wilcoxon p=0.0656, FIG. 2a, see Methods). Further, genealogical estimation of variant age (GEVA) suggests that ACE2 coding 146 variants are more recent (FIG. 3). Although ACE2 has been reported to be highly intolerant of loss-of-function variants (pLI=0.9977, gnomAD; FIG. 2h, see Methods) (Karczewski et al., 2019), we observed 5 predicted LOF singleton alleles (Table 1).

TABLE 1
ACE2 Variation in Human Population

| | | | | gnomAD |
|---|---|---|---|---|
| | | Protein | Allele | Allele |
| Position | Mutation | Consequence | Count | Frequency |
| 3 | S3N | p.SerAsn | 1 | 5.83625E−06 |
| 8 | L8F | p.Leu8Phe | 16 | 8.05814E−05 |
| 9 | L9P | p.Leu9Pro | 1 | 5.62943E−06 |
| 19 | S19P | p.Ser19Pro | 64 | 0.000312919 |
| 21 | I21V | p.Ile21Val | 2 | 1.0927E−05 |
| 21 | I21T | p.Ile21Thr | 1 | 5.46254E−06 |
| 23 | E23K | p.Glu23Lys | 1 | 5.45777E−06 |
| 26 | K26R | p.Lys26Arg | 797 | 0.003883296 |
| 26 | K26E | p.Lys26Glu | 1 | 5.45476E−06 |
| 27 | T27A | p.Thr27Ala | 2 | 1.09099E−05 |
| 31 | KB1R | p.Lys31Arg | 1 | 0 |
| 33 | N33I | p.Asn33Ile | 1 | 0 |
| 34 | H34R | p.His34Arg | 1 | 0 |
| 35 | E35K | p.Glu35Lys | 3 | 1.636E−05 |
| 37 | E37K | p.Glu37Lys | 8 | 3.89708E−05 |
| 38 | D38V | p.Asp38Val | 1 | 0 |
| 40 | F40L | p.Phe40Leu | 2 | 3.0862E−05 |
| 43 | S43N | p.Ser43Asn | 2 | 0 |
| 43 | S43R | p.Ser43Arg | 1 | 5.46185E−06 |
| 50 | Y50F | p.Tyr50Phe | 1 | 5.4817E−06 |
| 51 | N51D | p.Asn51Asp | 2 | 5.48501E−06 |
| 51 | N515 | p.Asn51Ser | 1 | 5.48731E−06 |
| 53 | N53S | p.Asn53Ser | 1 | 0 |
| 55 | T55A | p.Thr55Ala | 1 | 5.52312E−06 |
| 58 | N58H | p.Asn58His | 2 | 1.11702E−05 |
| 58 | N58K | p.Asn58Lys | 2 | 1.11867E−05 |
| 60 | Q60R | p.Gln60Arg | 2 | 1.12455E−05 |
| 62 | M62I | p.Met62Ile | 1 | 0 |
| 62 | M62V | p.Met62Val | 1 | 5.66646E−06 |
| 64 | N64K | p.Asn64Lys | 3 | 1.4664E−05 |
| 68 | K68E | p.Lys68Glu | 2 | 1.09457E−05 |
| 72 | F72V | p.Phe72Val | 1 | 5.47411E−06 |
| 80 | A80G | p.Ala80Gly | 1 | 0 |
| 82 | M82I | p.Met82Ile | 5 | 2.44178E−05 |
| 83 | Y83H | p.Tyr83His | 2 | 0 |
| 84 | P84T | p.Pro84Thr | 1 | 5.4707E−06 |
| 86 | Q86R | p.Gln86Arg | 2 | 1.09411E−05 |
| 92 | T92I | p.Thr92Ile | 2 | 1.09557E−05 |
| 102 | Q102P | p.Gln102Pro | 3 | 1.47451E−05 |
| 103 | N103H | p.Asn103His | 4 | 1.96661E−05 |
| 107 | V107A | p.Val107Ala | 2 | 1.10552E−05 |
| 113 | S113N | p.Ser113Asn | 1 | 0 |
| 115 | R115Q | p.Arg115Gln | 34 | 0.000170308 |
| 128 | S128T | p.Ser128Thr | 1 | 5.73809E−06 |
| 138 | P138A | p.Pro138Ala | 1 | 5.73283E−06 |
| 141 | C141Y | p.Cys141Tyr | 1 | 5.8307E−06 |
| 154 | N154K | p.Asnl54Lys | 2 | 1.09536E−05 |
| 158 | Y158H | p.Tyr158His | 1 | 0 |
| 159 | N159S | p.Asn159Ser | 3 | 1.63682E−05 |
| 163 | W163R | p.Trpl63Arg | 1 | 5.45557E−06 |
| 154 | A164S | p.Ala164Ser | 1 | 5.45461E−06 |
| 166 | E166Q | p.Glu166Gln | 1 | 5.4536E−06 |
| 171 | E171D | p.Glu171Asp | 1 | 4.55519E−05 |
| 171 | E171V | p.Glu171Val | 1 | 5.45411E−06 |
| 173 | G173S | p.Gly173Ser | 4 | 2.18172E−05 |
| 177 | R177S | p.Arg177Ser | 1 | 0 |
| 178 | P178L | p.Pro178Leu | 1 | 5.455E−06 |
| 184 | V184A | p.Val184Ala | 8 | 4.36503E−05 |
| 184 | V184G | p.Val184Gly | 1 | 4.58358E−05 |
| 186 | L186S | p.Leu186Ser | 1 | 5.4615E−06 |
| 190 | M190T | p.Met190Thr | 1 | 5.46591E−06 |
| 191 | A191P | p.Ala191Pro | 1 | 5.46819E−06 |
| 193 | A193E | p.Ala193Glu | 3 | 1.64126E−05 |
| 195 | H195N | p.His195Asn | 1 | 5.47627E−06 |
| 195 | H195Y | p.His195Tyr | 1 | 5.47627E−06 |
| 198 | D198N | p.Asp198Asn | 1 | 5.88166E−06 |
| 199 | Y199H | p.Tyr199His | 1 | 0 |
| 199 | Y199C | p.Tyr199Cys | 3 | 1.68777E−05 |
| 204 | R204T | p.Arg204Thr | 1 | 5.48152E−06 |
| 206 | D206G | p.Asp206Gly | 61 | 0.000299999 |
| 207 | Y207C | p.Tyr207Cys | 1 | 5.46866E−06 |
| 209 | V209I | p.Val209Ile | 1 | 5.46735E−06 |
| 211 | G211R | p.Gly211Arg | 261 | 0.001279889 |
| 216 | D216Y | p.Asp216Tyr | 1 | 5.46015E−06 |
| 216 | D216E | p.Asp216Glu | 3 | 1.47049E−05 |
| 219 | R219H | p.Arg219His | 18 | 9.83128E−05 |
| 219 | R219C | p.Arg219Cys | 71 | 0.000348058 |
| 220 | G220S | p.Gly220Ser | 3 | 1.63894E−05 |
| 225 | D225G | p.Asp225Gly | 1 | 0 |
| 229 | T229I | p.Thr229Ile | 1 | 5.4787E−06 |
| 239 | H239Q | p.His239Gln | 1 | 5.85967E−06 |
| 241 | H241Q | p.His241Gln | 1 | 0 |
| 242 | A242V | p.Ala242Val | 3 | 1.7254E−05 |
| 246 | A246T | p.Ala246Thr | 2 | 0 |
| 246 | A246S | p.Ala246Ser | 1 | 5.68311E−06 |
| 252 | Y252C | p.Tyr252Cys | 2 | 1.12827E−05 |
| 257 | S257N | p.Ser257Asn | 5 | 2.57054E−05 |
| 259 | I259T | p.Ile259Thr | 2 | 1.03069E−05 |
| 263 | P263S | p.Pro263Ser | 11 | 5.78415E−05 |
| 266 | 1266F | p.Leu266Phe | 1 | 0 |
| 270 | M270V | p.Met270Val | 5 | 2.98299E−05 |
| 280 | S280Y | p.Ser280Tyr | 1 | 5.65141E−06 |
| 282 | T282S | p.Thr282Ser | 2 | 0 |
| 287 | Q287R | p.Gln287Arg | 1 | 5.66046E−06 |
| 287 | Q287K | p.Gln287Lys | 1 | 5.65384E−06 |
| 290 | N290H | p.Asn290His | 2 | 1.13323E−05 |
| 291 | I291K | p.Ile291Lys | 3 | 1.70594E−05 |
| 292 | D292N | p.Asp292Asn | 2 | 1.13967E−05 |
| 295 | D295G | p.Asp295Gly | 8 | 4.6594E−05 |
| 297 | M297I | p.Met297Ile | 1 | 5.86304E−06 |
| 297 | M297L | p.Met297Leu | 1 | 5.85144E−06 |
| 300 | Q300R | p.Gln300Arg | 2 | 1.18694E−05 |
| 303 | D303N | p.Asp303Asn | 2 | 1.20266E−05 |
| 308 | F308L | p.Phe308Leu | 1 | 5.73365E−06 |
| 312 | E312K | p.Glu312Lys | 2 | 1.13409E−05 |
| 314 | F314S | p.Phe314Ser | 2 | 0 |
| 318 | V318A | p.Val318Ala | 2 | 0 |
| 326 | G326E | p.Gly326Glu | 1 | 5.51675E−06 |
| 329 | E329G | p.Glu329Gly | 7 | 3.44298E−05 |
| 332 | M332L | p.Met332Leu | 3 | 1.65432E−05 |
| 337 | G337R | p.Gly337Arg | 1 | 5.53168E−06 |
| 338 | N338S | p.Asn338Ser | 3 | 1.65937E−05 |
| 339 | V339G | p.Val339Gly | 1 | 5.53661E−06 |
| 341 | K341R | p.Lys341Arg | 81 | 0.000400192 |
| 346 | P346S | p.Pro346Ser | 1 | 5.59945E−06 |
| 352 | G352V | p.Gly352Val | 1 | 5.75218E−06 |
| 355 | 5355N | p.Asp355Asn | 2 | 1.17433E−05 |
| 360 | M360L | p.Met360Leu | 1 | 5.53836E−06 |
| 366 | M366I | p.Met366Thr | 2 | 1.09845E−05 |
| 368 | D368N | p.Asp368Asn | 1 | 5.49125E−06 |
| 374 | H374R | p.His374Arg | 1 | 5.47528E−06 |
| 375 | E375D | p.Glu375Asp | 3 | 1.64213E−05 |
| 377 | G377E | p.Gly377Glu | 1 | 5.47345E−06 |
| 378 | H378R | p.His378Arg | 18 | 8.79104E−05 |
| 383 | M383T | p.Met383Thr | 1 | 0 |
| 388 | Q388L | p.Gln383Leu | 4 | 2.18607E−05 |
| 389 | P389H | p.Pro389His | 7 | 3.82576E 05 |
| 397 | N397D | p.Asn397Asp | 3 | 1.46412E−05 |
| 398 | E398K | p.Glu398Lys | 1 | 5.46224E−06 |
| 405 | G405E | p.Gly405Glu | 1 | 5.46072E−06 |
| 413 | A413T | p.Ala413Thr | 1 | 0 |
| 417 | H417R | p.His417Arg | 1 | 0 |
| 419 | K419T | p.Lys419Thr | 1 | 5.46263E−06 |
| 420 | S420P | p.Ser420Pro | 1 | 4.54876E−05 |
| 421 | I421T | p.Ile421Thr | 1 | 5.46379E−06 |
| 426 | P426A | p.Pro426Ala | 1 | 5.46866E−06 |
| 427 | D427N | p.Asp427Asn | 2 | 0 |
| 427 | D427Y | p.Asp427Tyr | 2 | 1.0948E−05 |
| 437 | N437H | p.Asn437His | 1 | 0 |
| 437 | N437S | p.Asn437Ser | 1 | 0 |
| 445 | T445M | p.Thr445Met | 1 | 5.86937E−06 |
| 446 | I446M | p.Ile446Met | 1 | 4.53968E−05 |
| 447 | V447F | p.Val447Phe | 13 | 6.68439E−05 |
| 448 | G448E | p.Gly448Glu | 1 | 5.77481E−06 |
| 450 | L450V | p.Leu450Val | 1 | 5.71984E−06 |
| 455 | M455I | p.Met455Ile | 1 | 5.65163E−06 |
| 461 | W461R | p.Trp461Arg | 1 | 5.59851E−06 |
| 463 | V463I | p.Val463Ile | 1 | 5.57479E−06 |
| 466 | G466W | p.Gly466Trp | 1 | 5.57336E−06 |
| 467 | E467K | p.Glu467Lys | 4 | 2.24252E−05 |
| 468 | I468V | p.Ile468Val | 168 | 0.000838441 |
| 481 | K481N | p.Lys481Asn | 1 | 0 |
| 482 | R482Q | p.Arg482Gln | 3 | 1.7609E−05 |
| 483 | E483Q | p.Glu483Gln | 1 | 0 |
| 483 | E483D | p.Glu483Asp | 7 | 4.63908E−05 |
| 488 | V488A | p.Val488Ala | 1 | 6.32495E−06 |
| 491 | V491M | p.Val491Met | 1 | 6.28828E−06 |
| 494 | D494V | p.Asp494Val | 8 | 4.9578E−05 |
| 497 | Y497H | p.Tyr497His | 1 | 0 |
| 501 | A501T | p.Ala501Thr | 4 | 2.21659E−05 |
| 504 | F504I | p.Phe504Ile | 2 | 1.26824E−05 |
| 504 | F504L | p.Phe504Leu | 1 | 4.6307E−05 |
| 506 | V506A | p.Val506Ala | 1 | 6.56052E−06 |
| 509 | D509Y | p.Asp509Tyr | 1 | 0 |
| 510 | Y510H | p.Tyr510His | 1 | 6.86304E−06 |
| 511 | S511P | p.Ser511Pro | 1 | 0 |
| 518 | R518T | p.Arg518Thr | 1 | 5.4904E−06 |
| 519 | T519I | p.Thr519Ile | 1 | 5.48276E−06 |
| 521 | Y521H | p.Tyr521His | 1 | 5.47312E−06 |
| 527 | E527V | p.Glu527Val | 1 | 0 |
| 532 | A532T | p.Ala532Thr | 10 | 5.46305E−05 |
| 534 | K534R | p.Lys534Arg | 1 | 5.46138E−06 |
| 538 | P538L | p.Pro538Leu | 1 | 5.46379E−06 |
| 541 | K541N | p.Lys541Asn | 1 | 0 |
| 541 | K541I | p.Lys541Ile | 2 | 9.75895E−06 |
| 544 | I544N | p.Ile544Asn | 1 | 5.47714E−06 |
| 546 | N546D | p.Asn546Asp | 8 | 3.91654E−05 |
| 546 | N546S | p.Asn546Ser | 1 | 5.48585E−06 |
| 547 | S547C | p.Ser547Cys | 43 | 0.00021059 |
| 547 | S547F | p.Ser547Phe | 1 | 5.49058E−06 |
| 550 | A550G | p.Ala550Gly | 2 | 0 |
| 553 | K553T | p.Lys553Thr | 2 | 9.84518E−06 |
| 559 | R559S | p.Arg559Ser | 1 | 5.6744E−06 |
| 563 | S563L | p.Ser563Leu | 1 | 5.6211E−06 |
| 565 | P565T | p.Pro565Thr | 1 | 0 |
| 565 | P565S | p.Pro565Ser | 1 | 5.57588E−06 |
| 567 | T567A | p.Thr567Ala | 1 | 5.55222E−06 |
| 570 | L570S | p.Leu570Ser | 1 | 4.59876E−05 |
| 573 | V573A | p.Val573Ala | 2 | 1.09806E−05 |
| 574 | V574I | p.Val574Ile | 1 | 5.49082E−06 |
| 574 | V574A | p.Val574Ala | 1 | 0 |
| 575 | G575R | p.Gly575Arg | 1 | 5.48799E−06 |
| 582 | R582K | p.Arg582Lys | 3 | 1.64348E 05 |
| 582 | R582S | p.Arg582Ser | 3 | 1.46802E−05 |
| 585 | L585P | p.Leu585Pro | 1 | 5.47948E−06 |
| 586 | N586Y | p.Asn586Tyr | 2 | 9.78852E−06 |
| 588 | F588S | p.Phe588Ser | 1 | 5.48252E−06 |
| 589 | E589G | p.Glu589Gly | 1 | 5.48477E−06 |
| 593 | T593N | p.Thr593Asn | 3 | 1.47442E−05 |
| 595 | L595V | p.Leu595Val | 3 | 1.65411E−05 |
| 597 | D597E | p.Asp597Glu | 25 | 0.000123366 |
| 608 | T608I | p.Thr608Ile | 1 | 5.67617E−06 |
| 609 | D609N | p.Asp609Asn | 3 | 1.71863E−05 |
| 612 | P612L | p.Pro612Leu | 2 | 0 |
| 614 | A614S | p.Ala614Ser | 35 | 0.00017115 |
| 614 | A614T | p.Ala614Thr | 1 | 0 |
| 615 | D615G | p.Asp615Gly | 5 | 2.73928E−05 |
| 627 | A627V | p.Ala627Val | 2 | 1.0948E−05 |
| 628 | L628P | p.Leu628Pro | 1 | 0 |
| 629 | G629R | p.Gly629Arg | 1 | 0 |
| 629 | G629V | p.Gly629Val | 1 | 5.47411E−06 |
| 630 | D630H | p.Asp630His | 3 | 1.64245E−05 |
| 633 | Y633C | p.Tyr633Cys | 1 | 5.93384E−06 |
| 637 | D637N | p.Asp637Asn | 3 | 0 |
| 638 | N638S | p.Asn638Ser | 49 | 0.000253484 |
| 638 | N638D | p.Asn638Asp | 1 | 0 |
| 644 | R644Q | p.Arg644Gln | 1 | 0 |
| 646 | S646F | p.Ser646Phe | 1 | 0 |
| 652 | R652K | p.Arg652Lys | 1 | 5.75844E−06 |
| 653 | Q653K | p.Gln653Lys | 1 | 0 |
| 654 | Y654S | p.Tyr654Ser | 1 | 4.55208E−05 |
| 658 | V658I | p.Val658Ile | 1 | 5.95575E−06 |
| 660 | N660S | p.Asn660Ser | 1 | 5.99797E−06 |
| 664 | L664I | p.Leu664Ile | 2 | 0 |
| 665 | F665C | p.Phe665Cys | 1 | 0 |
| 667 | E667V | p.Glu667Val | 1 | 5.52077E−06 |
| 668 | E668K | p.Glu668Lys | 4 | 2.19972E−05 |
| 671 | R671Q | p.Arg671Gln | 4 | 1.96187E−05 |
| 671 | R671P | p.Arg671Pro | 1 | 4.61553E−05 |
| 672 | V672A | p.Val672Ala | 1 | 5.48195E−06 |
| 672 | V672L | p.Val672Leu | 2 | 1.09705E−05 |
| 673 | A673G | p.Ala673Gly | 1 | 5.47921E−06 |
| 673 | A673V | p.Ala673Val | 1 | 5.47921E−06 |
| 676 | K676E | p.Lys676Glu | 1 | 0 |
| 677 | P677L | p.Pro677Leu | 1 | 5.47525E−06 |
| 681 | F681V | p.Phe681Val | 1 | 5.47202E−06 |
| 688 | P688R | p.Pro688Arg | 1 | 5.47282E−06 |
| 689 | K689E | p.Lys689Glu | 3 | 1.64049E−05 |
| 690 | N690S | p.Asn690Ser | 1 | 5.47211E−06 |
| 692 | S692P | p.Ser692Pro | 115 | 0.000561883 |
| 693 | D693G | p.Asp693Gly | 1 | 5.47217E−06 |
| 696 | P696T | p.Pro696Thr | 2 | 1.09609E−05 |
| 697 | R697G | p.Arg697Gly | 46 | 0.000252134 |
| 703 | A703T | p.Ala703Thr | 1 | 0 |
| 703 | A703S | p.Ala703Ser | 3 | 1.65621E−05 |
| 706 | M706I | p.Met706Ile | 1 | 7.08572E−06 |
| 708 | R708Q | p.Arg708Gln | 1 | 6.91247E−06 |
| 708 | R708W | p.Arg708Trp | 3 | 1.80397E−05 |
| 709 | S709R | p.Ser709Arg | 1 | 6.88108E−06 |
| 710 | R710C | p.Arg710Cys | 5 | 2.89195E−05 |
| 710 | R710H | p.Arg710His | 7 | 3.99657E−05 |
| 716 | R716H | p.Arg716His | 15 | 8.18027E−05 |
| 716 | R716C | p.Arg716Cys | 1 | 6.19149E−06 |
| 719 | D719E | p.Asp719Glu | 1 | 6.01525E−06 |
| 720 | N720D | p.Asn720Asp | 3054 | 0.016215011 |
| 720 | N720S | p.Asn720Ser | 1 | 6.00402E−06 |
| 726 | G726R | p.Gly726Arg | 2 | 1.15294E−05 |
| 726 | G726E | p.Gly726Glu | 1 | 5.76558E−06 |
| 729 | P729L | p.Pro729Leu | 2 | 1.00837E−05 |
| 730 | T730K | p.Thr730Lys | 1 | 5.63146E−06 |
| 731 | L731F | p.Leu731Phe | 286 | 0.001434915 |
| 733 | P733L | p.Pro733Lcu | 2 | 0 |
| 734 | P734L | p.Pro734Leu | 2 | 1.12724E−05 |
| 735 | N735K | p.Asn735Lys | 1 | 5.64127E−06 |
| 737 | P737A | p.Pro737Ala | 1 | 5.64723E−06 |
| 737 | P737L | p.Pro737Leu | 2 | 0 |
| 740 | S740P | p.Ser740Pro | 1 | 4.59749E−05 |
| 741 | I741V | p.Ile741Val | 20 | 0.000100345 |
| 745 | V745I | p.Val745Ile | 2 | 1.01287E−05 |
| 751 | G751E | p.Gly751Glu | 2 | 1.1659E−05 |
| 752 | V752M | p.Val752Met | 1 | 0 |
| 753 | I753M | p.Ile753Met | 1 | 5.83216E−06 |
| 761 | I761V | p.Ile761Val | 1 | 6.28943E−06 |
| 767 | D767H | p.Asp767His | 2 | 1.34282E−05 |
| 768 | R768W | p.Arg768Trp | 2 | 1.38522E−05 |
| 769 | K769E | p.Lys769Glu | 1 | 6.92919E−06 |
| 771 | K771R | p.Lys771Arg | 1 | 4.53762E−05 |
| 772 | N772S | p.Asn772Ser | 12 | 6.02101E−05 |
| 774 | A774G | p.Ala774Gly | 1 | 5.61605E−06 |
| 774 | A774P | p.Ala774Pro | 1 | 5.61549E−06 |
| 774 | A774T | p.Ala774Thr | 1 | 5.61549E−06 |
| 776 | S776R | p.Ser776Arg | 1 | 4.55021E−05 |
| 781 | V781H | p.Tyr781His | 3 | 1.4797E−05 |
| 782 | A782V | p.Ala782Val | 11 | 6.07839E−05 |
| 785 | D785N | p.Asp785Asn | 7 | 3.86678E−05 |
| 793 | P793L | p.Pro793Leu | 1 | 5.57324E−06 |
| 796 | Q796R | p.Gln796Arg | 2 | 1.11324E−05 |
| 801 | V801G | p.Val801Gly | 1 | 5.61728E−06 |
| 802 | Q802R | p.Gln802Arg | 1 | 0 |
| 804 | S804F | p.Ser804Phe | 1 | 5.80124E−06 |
| 805 | F805I | p.Phe805Ile | 4 | 2.3209E−05 |
| 482 | R482* | p.Arg482Ter | 1 | 0 |
| 244 | V244fs | p.Val244LeufsTer27 | 1 | 0 |
| 656 | L656* | p.Leu656Ter | 1 | 5.9153E−06 |
| 405 | G405del | p.Gly405del | 2 | 1.09215E−05 |
| 422 | G422fs | p.Gly422ValfsTer15 | 1 | 5.46472E−06 |
| 116 | L116* | p.Leu116Ter | 1 | 6.43882E−06 |
| 313 | K313del | p.Lys313del | 1 | 5.642E−06 |

Structural studies involving SARS-CoV and SARS-CoV-2 S-protein and complex with human ACE2 have identified three regions in an ˜120 amino acid claw-like exposed outer surface of the human ACE2 (ACE2-claw) that contributes to its binding to the S-protein (Shang et al., 2020; Walls et al., 2020; Wrapp et al., 2020; Yan et al., 2020). The key residues at the ACE-2 S-protein-RBD interface include S19, Q24, T27, F28, D30, K31, H34, E35, E37, D38, Y41, Q42, L45, L79, M82, Y83, T324, Q325, G326, E329, N330, K353, G354, D355, R357, P389, and R393 (FIG. 1e). Mutagenesis of four residues, namely M82, Y83, P84 and K353, in the S-protein-binding interface of rat ACE2 was sufficient to convert rat ACE2 into a human SARS-CoV receptor, further indicating the importance of this region in determining the host range and specificity of CoVs (Li et al., 2005b). Considering these findings, we focused on variants within the human ACE2-claw S-protein RBD-binding interface and identified protein alterations in 44 codons that resulted in 49 unique variants for a total of 968 allelic variants. This included K26R, the second most frequent human ACE2 protein-altering variant (0.4% allele frequency; allele count=797, gnomAD), S19P, T27A, K31R, N33I, H34R, E35K, E37K, D38V, N51S, N64K, K68E, F72V, T92I, Q102P, G326E, G352V, D355N, H378R, Q388L, and D509Y (FIGS. 1b and c; FIG. 18). These variants could potentially increase or decrease the binding affinity of ACE2 to the S-protein and thereby alter the ability of the virus to infect the host cell.

### Structural Evaluation of ACE2 Polymorphism

To investigate the effect of the ACE2 polymorphisms on receptor recognition by the SARS-CoV-2 RBD, we modeled the identified ACE2 variants using published cryo-EM and crystal structures of ACE2/SARS-CoV-2 RBD complexes (Shang et al., 2020; Walls et al., 2020; Wrapp et al., 2020; Yan et al., 2020). Based on the evaluation of the structures and a functional analysis of a synthetic human ACE2 mutant library for RBD binding affinity (Chan et al., 2020b), we broadly classified ACE2 polymorphic variants into two categories with respect to their predicted effect on ACE2-RBD binding as enhancing or disrupting (FIG. 18). These two groups of polymorphic variants mapped onto the ACE2 structure remarkably segregate into two distinct clusters at the ACE2/CoV-2 RBD interface (FIG. 18a). The predicted enhancing variants cluster to the ACE2 surface most proximal to the receptor-binding ridge of CoV-2 RBD (FIG. 18b) whereas the majority of the predicted disrupting variants reside centrally on the two major ACE2 α-helices that substantially contribute to the buried surface area at the interface (FIG. 18b). The spatial segregation of the functionally different ACE2 variants can be structurally explained. The loop conformation in the receptor-binding ridge differs significantly in SARS-CoV-2 from that of SARS-CoV owing to the presence of bulky residues (V483 and E484) in the loop (Shang et al., 2020). This feature allows the SARS-CoV-2 loop to extend further towards ACE2 establishing more extensive contacts with the receptor (FIG. 18a). Hence, natural ACE2 variants in this region could be exploited by the CoV-2 loop, increasing susceptibility to viral infection. In contrast, most interactions that CoV-2 makes with the core of the ACE2 interface are centered on two α-helices (α1 and α2) and are not unique to CoV-2. They encompass what seem to be critical binding hotspots, and thus centrally located polymorphic variants are more likely to reduce viral recognition.

### Altered Affinity of ACE2 Variants for SARS-CoV-2 S-Protein

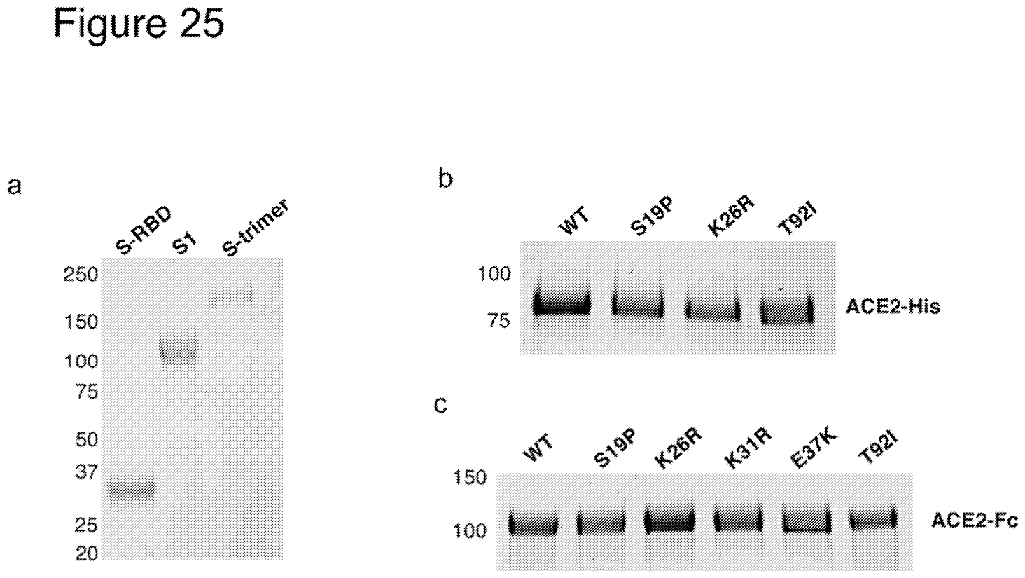

To validate our structural predictions, we measured the effect of select ACE2 polymorphisms on its binding affinity to CoV-2 S-protein. We expressed and purified the S1 subunit of the S-protein, CoV-2 S-RBD, and a trimer stabilized form of S-protein (S-trimer; FIG. 25). We also recombinantly produced His-tagged monomeric and Fc-tagged dimeric forms of the extracellular domain of wildtype ACE2 (WT) and variant forms of ACE2 (S19P, K26R, K31R, E37K and T92I; FIG. 25). These variants were selected based on their population frequency and the predicted effect on their interaction with S-protein.

We tested the affinity of these ACE2 variants to a panel of S-protein constructs using an enzyme-linked immunosorbent assay (ELISA). We used dimeric ACE2-Fc to assess its binding to the S-protein variants. We found the ACE2-Fc WT dimer bound to the isolated S-RBD (EC50 1.01 nM) and S-trimer (EC50 0.95 nM) more strongly compared to the S1 subunit (EC50 10.4 nM) (Table 3). This is consistent with previous studies that showed a decreased ACE2 affinity for SARS-CoV S1 subunit compared to S-RBD, indicating a conformational difference between these variants (Hoffmann et al., 2020; Li et al., 2005a; Wong et al., 2004). In the trimeric state, in contrast to the monomeric full length S1-protein, the RBD within the S1 subunit in one or more of the constituent S-proteins is known to adopt a receptor-accessible ‘RBD-out’confirmation, supporting its high affinity for ACE2 that is comparable to that observed for isolated RBD (Walls et al., 2019; Wrapp et al., 2020; Yan et al., 2020).

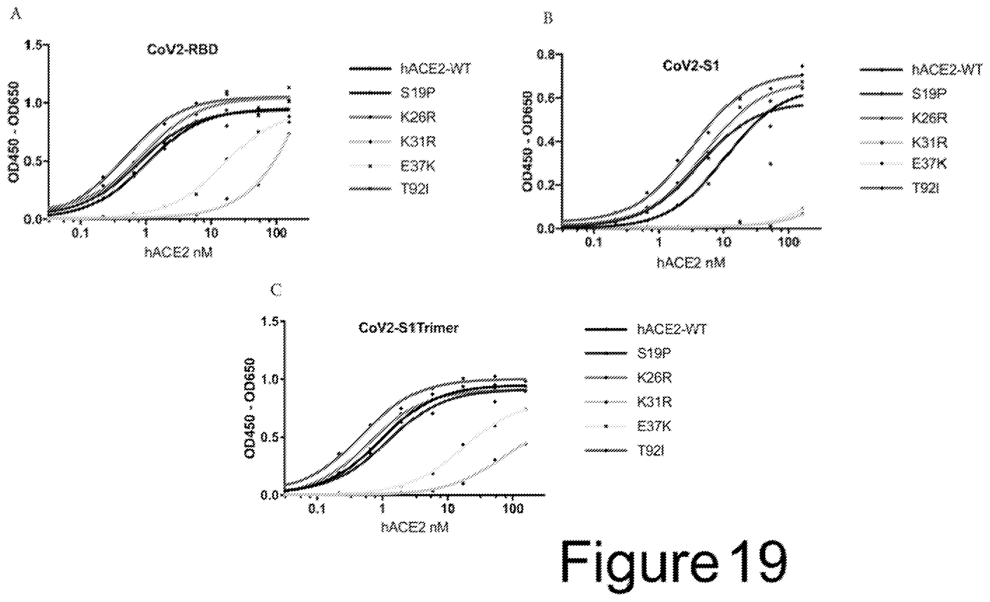

The affinity of the S-RBD or S-trimer for ACE2-Fc variants based on ELISA is shown in Table 1 and FIGS. 19-22. As can be seen from the table and figures, ACE2 variants with a single amino acid substitution, S19P, K26R, N90E or T92I, have EC50 values significantly lower than the EC50 value of WT human ACE2 protein. The T92I, glycosylation site mutant of ACE2-Fc, showed an increased affinity for S-RBD (EC50 0.48 nM) and S-trimer (EC50 0.47 nM). In contrast, ACE2 variants with either N33I, H34R, A80G or N90T bind the different forms of SARS-CoV-2 S-proteins with an binding affinity around that of WT ACE2 protein or slightly lower; whereas, presence of either K31R or E37K substitution results in a dramatic drop in affinity for SARS-CoV-2 S-protein, at least one order of magnitude to possibly 2 orders of magnitude. As, observed with E37K (EC50 15.8 nM for S-RBD and EC50 17.6 nM for S-trimer), K31R ACE2-Fc had a decreased affinity for S-RBD (EC50=298 nM) and S-trimer (EC50=73 nM) when compared to WT ACE2-Fc (EC50 1.01 nM for S-RBD and EC50 0.95 nM for S-trimer).

A recent mutagenesis screen using a synthetic human ACE2 mutant library identified variants that either increased or decreased its binding to SARS-CoV-2 S-protein (Procko, 2020). Using a sequencing-based enrichment assay, the fold enrichment or depletion of the mutant sequences was measured in this study (Procko, 2020). Mapping the enrichment z-scores from this study (Procko, 2020) to the spectrum of natural ACE2 polymorphisms, we identified several rare ACE2 variants (FIG. 1c) that likely alter their binding to the SARS-CoV-2 S-protein and thereby protect or render individuals more susceptible to the virus. The majority of the variants that were predicted to alter the interaction between ACE2 and the virus S-protein were clustered around the N-terminal region of ACE2 that interacts with the S-protein (FIG. 1e).

Included among the ACE2 polymorphic variants that increase ACE2/S-protein interaction are S19P, I21T/V, E23K, A25T, K26E or K26R, T27A, N33, F40L, N64K, Q60R, N64K, W69C, A80G, T92I, Q102P, Q325R, M366T, D367V, 1-1374R, H378R, M383T, E398D, E398K, T445M, I446M, and Y510H. Among these, the T92I polymorphism stands out in particular because it is part of a NxT/S (where x is any amino acid except proline) consensus N-glycosylation motif (Gavel and von Heijne, 1990) where N90 is the site of N-glycan addition. The ACE2 NxT/S motif, while conserved in 96 out of 296 jawed vertebrate with ACE2 sequence available is absent or altered in several species, including the civet cat (Paguma larvata) and several bat species where residue N90 is mutated, a proline is present at position 91 or the T92 is altered to any amino acid except serine (FIG. 1d, FIG. 3 and Table 2) (Demogines et al., 2012; Gavel and von Heijne, 1990; Li et al., 2005b). These ACE2 variations are expected to abolish glycosylation at N90 (Gavel and von Heijne, 1990). Furthermore, a mutation that altered the NxT/S motif in human ACE2 to a civet ACE2-like sequence (90-NLTV-93 to DAK1), expected to abolish the N-glycosylation, increased the SARS-CoV infectivity and S-protein binding (FIG. 1d) (Li et al., 2005b). The T92I mutant we identified showed a strong enrichment in the sequencing-based screen for S-protein binders (Procko, 2020). Considering these observations, we conclude that the T92I mutation increases the ACE2/S-protein binding affinity rendering individuals harboring this mutation more susceptibility to the virus.

TABLE 2
Conservation of N90 Glycosylation Motif in Annotated Jawed Vertebrate ACE2
Orthologs

| | | | | | | | Motif |
|---|---|---|---|---|---|---|---|
| | | Residue | Residue | Residue | Residue | Residue | NxT/S |
| Common name | Refseq ID | #90 | #90 | #91 | #92 | #93 | present? |
| Human | NP_001358344.1 | Q | N | L | T | V | Yes |
| house mouse | NP_081562.2 | Q | T | P | I | I | No |
| Norway rat | NP_001012006.1 | Q | N | A | T | I | No |
| zebrafish | XP_005169416.1 | S | D | P | I | I | No |
| pig | NP_001116542.1 | Q | T | L | I | L | No |
| Rhesus monkey | NP_001129168.1 | Q | N | L | T | V | Yes |
| cattle | XP_005228485.1 | Q | N | L | T | L | Yes |
| dog | NP_001158732.1 | Q | D | S | T | V | No |
| rabbit | XP_002719891.1 | Q | N | L | T | V | Yes |
| tropical clawed | XP_002938293.2 | T | D | P | S | I | No |
| frog | | | | | | | |
| chicken | XP_416822.2 | Q | D | A | V | T | No |
| chimpanzee | XP_016798468.1 | Q | N | L | T | V | Yes |
| domestic cat | XP_023104564.1 | H | N | T | T | V | Yes |
| sheep | XP_011961657.1 | Q | N | L | T | L | Yes |
| rainbow trout | XP_021433278.1 | S | D | P | L | I | No |
| Atlantic cod | XP_030232530.1 | K | D | P | V | V | No |
| giant panda | XP_002930657.1 | H | N | S | T | V | Yes |
| Brandt's bat | XP_014399780.1 | Q | N | L | T | I | Yes |
| elephant shark | XP_007889845.1 | S | D | N | I | I | No |
| domestic ferret | NP_001297119.1 | Q | D | P | I | I | No |
| golden hamster | XP_005074266.1 | Q | N | L | T | I | Yes |
| naked mole-rat | XP_004866157.1 | Q | N | L | T | V | Yes |
| wild yak | XP_005903173.1 | Q | N | L | T | L | Yes |
| barramundi perch | XP_018539189.1 | K | D | Q | E | I | No |
| white-tufted-ear | XP_008987241.1 | Q | N | L | T | V | Yes |
| marmoset | | | | | | | |
| horse | XP_001490241.1 | Q | N | L | T | V | Yes |
| greater | XP_022605054.1 | K | N | P | E | I | No |
| amberjack | | | | | | | |
| turquoise killifish | XP_015808977.1 | K | D | P | E | V | No |
| two-lined caecilian | XP_029459086.1 | T | E | P | E | I | No |
| Microcaecilia | XP_030058174.1 | T | D | P | E | T | No |
| unicolor | | | | | | | |
| Mexican tetra | XP_022523929.1 | S | D | E | L | V | No |
| coelacanth | XP_005997915.2 | T | D | P | H | I | No |
| spotted gar | XP_006639185.1 | A | D | K | K | I | No |
| Atlantic herring | XP_031414786.1 | N | D | L | E | I | No |
| goldfish | XP_026131313.1 | S | D | P | L | I | No |
| channel catfish | XP_017313836.1 | S | D | H | E | V | No |
| electric eel | XP_026867211.1 | T | D | P | E | I | No |
| northern pike | XP_010884777.1 | K | D | P | L | I | No |
| Atlantic salmon | XP_014062928.1 | . | . | . | . | . | NA |
| Arctic char | XP_023998967.1 | S | V | I | I | D | No |
| mummichog | XP_021178197.1 | K | D | P | Q | I | No |
| guppy | XP_008402714.1 | S | D | P | V | I | No |
| southern platyfish | XP_005799835.1 | N | D | P | V | I | No |
| Nile tilapia | XP_003445853.2 | N | D | L | E | I | No |
| Burton's | XP_005943362.1 | N | D | L | E | I | No |
| mouthbrooder | | | | | | | |
| eastern happy | XP_026020155.1 | N | D | L | E | I | No |
| yellow perch | XP_028441363.1 | K | D | P | E | I | No |
| gilthead | XP_030271236.1 | K | D | R | E | L | No |
| seabream | | | | | | | |
| black rockcod | XP_010790455.1 | T | D | A | T | I | No |
| Japanese | XP_019935235.1 | K | D | A | K | I | No |
| flounder | | | | | | | |
| Green sea turtle | XP_007070561.1 | M | D | P | I | V | No |
| Painted turtle | XP_023964517.1 | T | D | P | I | V | No |
| American | XP_019350687.1 | M | D | P | L | I | No |
| alligator | | | | | | | |
| Australian | XP_019384826.1 | . | D | P | V | I | No |
| saltwater | | | | | | | |
| crocodile | | | | | | | |
| mainland tiger | XP_026530754.1 | S | N | E | T | I | Yes |
| snake | | | | | | | |
| Pseudonaja | XP_026570054.1 | A | N | E | T | I | Yes |
| textilis | | | | | | | |
| emu | XP_025976560.1 | T | D | D | L | I | No |
| mallard | XP_012949915.2 | Q | D | P | L | L | No |
| chimney swift | XP_009992128.1 | S | D | A | L | I | No |
| rock pigeon | XP_021154486.1 | Q | D | D | L | T | No |
| peregrine falcon | XP_005231984.2 | Q | D | A | L | T | No |
| white-tailed eagle | XP_000025641.1 | Q | D | D | L | T | No |
| helmeted | XP_021240731.1 | Q | D | A | V | T | No |
| guineafowl | | | | | | | |
| Ring-necked | XP_031451919.1 | Q | D | A | A | T | No |
| pheasant | | | | | | | |
| turkey | XP_019467554.1 | Q | D | A | A | T | No |
| Common canary | XP_009087922.1 | K | D | D | L | T | No |
| Great Tit | XP_015486815.1 | T | D | D | L | T | No |
| Common starling | XP_014731370.1 | T | D | D | L | I | No |
| great cormorant | XP_009509070.1 | Q | D | A | L | T | No |
| emperor penguin | XP_009275140.1 | Q | D | A | L | T | No |
| Adelie penguin | XP_009323767.1 | Q | D | T | L | T | No |
| Anna's | XP_008492997.2 | T | D | A | L | I | No |
| hummingbird | | | | | | | |
| platypus | XP_001515597.2 | S | D | R | S | L | No |
| Tasmanian devil | XP_031814825.1 | S | A | Y | P | I | No |
| nine-banded | XP_004449124.1 | S | N | L | T | N | Yes |
| armadillo | | | | | | | |
| western | XP_007538670.1 | Q | N | P | T | V | No |
| European | | | | | | | |
| hedgehog | | | | | | | |
| small Madagascar | XP_004710002.1 | T | D | P | I | I | No |
| hedgehog | | | | | | | |
| black flying fox | XP_006911709.1 | Q | D | P | I | L | No |
| Egyptian rousette | XP_015974412.1 | Q | D | P | E | L | No |
| common vampire | XP_024425698.1 | K | D | V | N | V | No |
| bat | | | | | | | |
| Tufted capuchin | XP_032141854.1 | Q | N | L | T | V | Yes |
| sooty mangabey | XP_011891198.1 | Q | N | L | T | V | Yes |
| crab-eating | XP_005593094.1 | Q | N | L | T | V | Yes |
| macaque | | | | | | | |
| pig-tailed | XP_011733505.1 | Q | N | L | T | V | Yes |
| macaque | | | | | | | |
| olive baboon | XP_021788732.1 | Q | N | L | T | V | Yes |
| gelada | XP_025227847.1 | Q | N | L | T | V | Yes |
| drill | XP_011850923.1 | Q | N | L | T | V | Yes |
| western gorilla | XP_018874749.1 | Q | N | L | T | I | Yes |
| pygmy | XP_008972428.1 | Q | N | L | T | V | Yes |
| chimpanzee | | | | | | | |
| Sumatran | NP_001124604.1 | Q | N | L | T | V | Yes |
| orangutan | | | | | | | |
| red fox | XP_025842512.1 | Q | D | S | T | V | No |
| leopard | XP_019273508.1 | H | N | T | T | V | Yes |
| puma | XP_025790417.1 | H | N | T | T | V | Yes |
| California sea lion | XP_027465353.1 | Q | D | S | T | V | No |
| Pacific walrus | XP_004415448.1 | Q | D | S | T | V | No |
| Weddell seal | XP_030886750.1 | . | . | . | . | . | NA |
| harbor seal | XP_032245506.1 | Q | D | S | T | V | No |
| long-finned pilot | XP_030703991.1 | R | N | L | T | L | Yes |
| whale | | | | | | | |
| killer whale | XP_004269705.1 | R | N | L | T | L | Yes |
| common | XP_019781177.1 | R | N | L | T | L | Yes |
| bottlenose | | | | | | | |
| dolphin | | | | | | | |
| beluga whale | XP_022418360.1 | R | N | L | T | L | Yes |
| sperm whale | XP_023971279.1 | Q | N | L | T | L | Yes |
| African savanna | XP_023410960.1 | S | S | S | I | I | No |
| elephant | | | | | | | |
| ass | XP_014713133.1 | Q | N | L | T | V | Yes |
| Przewalski’s | XP_008542995.1 | Q | N | L | T | V | Yes |
| horse | | | | | | | |
| Bactrian camel | XP_010966303.1 | Q | N | V | T | L | Yes |
| Arabian camel | XP_010991717.1 | Q | N | V | T | L | Yes |
| Odocoileus | XP_020768965.1 | Q | N | L | T | L | Yes |
| virginianus | | | | | | | |
| texanus | | | | | | | |
| zebu cattle | XP_019811719.1 | Q | N | L | T | L | Yes |
| goat | NP_001277036.1 | Q | N | L | T | L | Yes |
| Malayan pangolin | XP_017505746.1 | Q | N | D | T | I | Yes |
| American pika | XP_004597549.2 | Q | N | L | T | T | Yes |
| Alpine marmot | XP_015343540.1 | Q | N | F | T | L | Yes |
| Arctic ground | XP_026252505.1 | Q | N | F | T | L | Yes |
| squirrel | | | | | | | |
| Ord's kangaroo | XP_012887572.1 | Q | N | P | I | L | No |
| rat | | | | | | | |
| Chinese hamster | XP_003503283.1 | Q | N | L | I | I | No |
| white-footed | XP_028743609.1 | P | N | L | I | I | No |
| mouse | | | | | | | |
| Ryukyu mouse | XP_021009138.1 | Q | T | P | I | I | No |
| shrew mouse | XP_021043935.1 | Q | N | P | V | I | No |
| domestic guinea | XP_023417808.1 | Q | N | L | T | V | Yes |
| pig | | | | | | | |
| degu | XP_023575315.1 | Q | N | L | T | V | Yes |
| gray short-tailed | XP_007500935.1 | T | N | A | T | V | Yes |
| opossum | | | | | | | |
| Chinese soft- | XP_006122891.1 | T | N | H | T | V | Yes |
| shelled turtle | | | | | | | |
| Bolivian squirrel | XP_010334925.1 | Q | N | L | T | V | Yes |
| monkey | | | | | | | |
| reedfish | XP_028655640.1 | S | N | Y | T | I | Yes |
| green anole | XP_008105455.1 | N | N | D | T | I | Yes |
| Cape elephant | XP_006892457.1 | S | D | P | S | I | No |
| shrew | | | | | | | |
| sheepshead | XP_015226730.1 | K | D | L | Q | I | No |
| minnow | | | | | | | |
| polar bear | XP_008694637.1 | H | N | S | T | V | Yes |
| big brown bat | XP_008153150.1 | Q | N | L | T | I | Yes |
| Hawaiian monk | XP_021536480.1 | Q | D | S | T | L | No |
| seal | | | | | | | |
| common wombat | XP_027691156.1 | S | D | P | Q | I | No |
| Opisthocomus | XP_009938970.1 | Q | D | A | L | T | No |
| hoazin | | | | | | | |
| Northern fulmar | XP_009574896.1 | H | D | A | L | T | No |
| Nothoprocta | XP_025891105.1 | K | N | D | L | I | No |
| perdicaria | | | | | | | |
| hybrid cattle | XP_027389727.1 | Q | N | L | T | L | Yes |
| alpaca | XP_006212709.1 | E | N | V | T | L | Yes |
| gray mouse | XP_020140826.1 | Q | N | L | T | I | Yes |
| lemur | | | | | | | |
| small-eared galago | XP_003791912.1 | Q | N | R | T | V | Yes |
| Indian medaka | XP_024150631.1 | K | D | P | E | I | No |
| torafugu | XP_029702274.1 | K | N | A | E | I | No |
| Monterrey | XP_027871671.1 | N | D | P | V | I | No |
| platyfish | | | | | | | |
| cheetah | XP_026910297.1 | H | N | T | T | V | Yes |
| long-tailed | XP_013362428.1 | Q | N | L | T | V | Yes |
| chinchilla | | | | | | | |
| northern fur seal | XP_025713397.1 | Q | D | S | T | V | No |
| Steller sea lion | XP_027970822.1 | Q | D | S | T | V | No |
| Western | XP_032082934.1 | T | N | E | T | I | Yes |
| terrestrial garter | | | | | | | |
| snake | | | | | | | |
| Thamnophis | XP_013926936.1 | T | N | E | T | I | Yes |
| sirtalis | | | | | | | |
| southern | XP_031226742.1 | K | T | P | I | I | No |
| multimammate | | | | | | | |
| mouse | | | | | | | |
| Dalmatian pelican | XP_009478920.1 | Q | D | D | L | T | No |
| ermine | XP_032187677.1 | Q | D | P | I | I | No |
| mangrove rivulus | XP_017295385.1 | H | D | P | T | V | No |
| meerkat | XP_029786256.1 | Q | N | T | T | V | Yes |
| red-throated loon | XP_009816127.1 | Q | D | A | L | I | Yes |
| Ma's night | XP_012290105.1 | Q | N | L | T | V | Yes |
| monkey | | | | | | | |
| koala | XP_020863153.1 | S | D | P | Q | I | No |
| Chinese alligator | XP_025066628.1 | . | D | P | L | I | No |
| narwhal | XP_029095804.1 | R | N | L | T | L | Yes |
| European shrew | XP_004612266.1 | T | D | P | K | V | No |
| red-bellied | XP_017550079.1 | S | D | P | L | I | No |
| piranha | | | | | | | |
| thirteen-lined | XP_005316051.3 | Q | N | F | T | L | Yes |
| ground squirrel | | | | | | | |
| Bison | XP_010833001.1 | Q | N | L | T | L | Yes |
| swamp eel | XP_020465646.1 | R | D | A | E | I | No |
| white-throated | XP_005491832.2 | T | D | E | L | T | No |
| sparrow | | | | | | | |
| Oreochromis | XP_031584810.1 | N | D | L | E | I | No |
| aureus | | | | | | | |
| Amazon molly | XP_007560208.1 | N | D | P | V | I | No |
| sailfin molly | XP_014895313.1 | N | D | P | V | I | No |
| Poecilia | XP_014837025.1 | N | D | P | V | I | No |
| mexicana | | | | | | | |
| medium ground- | XP_005426221.1 | T | D | E | L | T | No |
| finch | | | | | | | |
| killdeer | XP_009887331.1 | Q | D | P | L | I | No |
| lesser Egyptian | XP_004671523.1 | Q | N | P | T | I | No |
| jerboa | | | | | | | |
| bald eagle | XP_010579828.1 | Q | D | D | L | T | No |
| Austrofundulus | XP_013888928.1 | N | D | P | N | I | No |
| limnaeus | | | | | | | |
| brown roatelo | XP_010178703.1 | Q | D | P | L | I | No |
| Red-legged | XP_009703695.1 | Q | D | A | L | T | No |
| seriema | | | | | | | |
| sunbittern | XP_010156467.1 | Q | D | A | L | I | No |
| common cuckoo | XP_009563864.1 | Q | D | A | L | T | No |
| Bam owl | XP_009969209.1 | Q | D | A | L | T | No |
| Cottoperca gobio | XP_029283581.1 | R | D | S | T | I | No |
| ballan wrasse | XP_020493627.1 | T | I | P | E | I | No |
| rifleman | XP_009082150.1 | . | . | . | . | . | NA |
| bar-tailed trogon | XP_009867056.1 | G | D | D | L | I | No |
| speckled | XP_010206054.1 | . | . | . | . | . | NA |
| mousebird | | | | | | | |
| carmine bee- | XP_008937519.1 | Q | N | A | T | T | Yes |
| eater | | | | | | | |
| little brown bat | XP_023609437.1 | Q | N | S | T | I | Yes |
| zebra finch | XP_002194303.3 | A | D | D | P | T | No |
| collared | XP_005037422.1 | T | D | D | L | T | No |
| flycatcher | | | | | | | |
| green monkey | XP_007989304.1 | Q | N | L | T | V | Yes |
| Canada lynx | XP_030160839.1 | H | N | T | T | I | Yes |
| black snub-nosed | XP_017744069.1 | Q | N | L | T | V | Yes |
| monkey | | | | | | | |
| golden snub- | XP_010364367.2 | Q | N | L | T | V | Yes |
| nosed monkey | | | | | | | |
| | XP_003261132.2 | Q | N | L | T | I | Yes |
| flier cichlid | XP_030582139.1 | Q | D | L | E | I | No |
| climbing perch | XP_026233431.1 | S | D | P | E | I | No |
| Amur tiger | XP_007090142.1 | H | N | T | T | V | Yes |
| prairie vole | XP_005358818.1 | Q | N | L | L | L | No |
| spiny chromis | XP_022063988.1 | T | D | P | E | I | No |
| clown | XP_023124156.1 | K | D | P | E | I | No |
| anemonefish | | | | | | | |
| American crow | XP_017583883.1 | . | . | . | . | . | NA |
| Camarhynchus | XP_030811385.1 | T | D | E | L | T | No |
| parvulus | | | | | | | |
| water buffalo | XP_006041602.1 | Q | N | L | T | L | Yes |
| pale spear-nosed | XP_028378317.1 | T | D | V | T | V | No |
| bat | | | | | | | |
| Pacific white- | XP_026951598.1 | R | N | L | T | L | Yes |
| sided dolphin | | | | | | | |
| yellow-bellied | XP_027802308.1 | Q | N | F | T | L | Yes |
| marmot | | | | | | | |
| Japanese quail | XP_015742063.1 | Q | D | A | V | T | No |
| white-throated | XP_010217584.1 | K | D | D | L | I | No |
| tinamou | | | | | | | |
| Gharial | XP_019381060.1 | . | D | P | L | I | No |
| White-tailed | XP_010290019.1 | Q | D | A | L | T | No |
| tropicbird | | | | | | | |
| central bearded | XP_020642422.1 | S | N | E | T | I | Yes |
| dragon | | | | | | | |
| Protobothrops | XP_029140508.1 | T | N | E | T | I | Yes |
| mucrosquamatus | | | | | | | |
| zebra mbuna | XP_004543482.1 | N | D | L | E | I | No |
| tiger tail seahorse | XP_019742561.1 | K | D | P | Q | I | No |
| Asian | XP_018584732.1 | T | D | P | T | V | No |
| bonytongue | | | | | | | |
| saffron-crested | XP_027544864.1 | E | D | N | L | I | No |
| tyrant-manakin | | | | | | | |
| Ursus arctos | XP_026333865.1 | H | N | S | T | V | Yes |
| horribilis | | | | | | | |
| Downy | XP_009909849.1 | . | . | . | . | . | NA |
| woodpecker | | | | | | | |
| Yangtze River | XP_007466389.1 | Q | N | L | T | L | Yes |
| dolphin | | | | | | | |
| red-crested | XP_009978415.1 | Q | D | A | L | I | No |
| turaco | | | | | | | |
| Nanorana parkeri | XP_018418558.1 | T | D | E | M | L | No |
| crested ibis | XP_009474590.1 | Q | D | A | L | T | No |
| large flying fox | XP_011361275.1 | Q | D | P | I | L | No |
| star-nosed mole | XP_012585871.1 | Q | D | P | I | V | No |
| bicolor | XP_008290762.1 | K | D | P | E | I | No |
| damselfish | | | | | | | |
| Gekko japonicus | XP_015273067.1 | S | D | P | H | I | No |
| great blue- | XP_020781598.1 | K | D | R | E | I | No |
| spotted | | | | | | | |
| mudskipper | | | | | | | |
| blue tit | XP_023774184.1 | T | D | D | L | T | No |
| Siamese fighting | XP_028999570.1 | S | D | P | E | I | No |
| fish | | | | | | | |
| willow flycatcher | XP_027757151.1 | E | D | N | L | I | No |
| live sharksucker | XP_029354066.1 | K | D | P | E | I | No |
| Buceros | XP_010136813.1 | Q | H | D | L | T | No |
| rhinoceros | | | | | | | |
| silvestris | | | | | | | |
| Kea | XP_010012481.1 | . | . | . | . | . | NA |
| Burmese python | XP_007431942.2 | T | D | E | T | I | No |
| Tibetan ground-tit | XP_005516712.1 | T | D | D | L | T | No |
| jewelled blenny | XP_029949252.1 | S | D | P | E | I | No |
| Cape golden | XP_006835673.1 | S | N | S | T | I | Yes |
| mole | | | | | | | |
| great roundleaf | XP_019522936.1 | Q | N | A | T | I | Yes |
| bat | | | | | | | |
| Macqueen’s | XP_010120523.1 | Q | D | A | L | T | No |
| bustard | | | | | | | |
| cuckoo roller | XP_009954393.1 | Q | D | A | L | T | No |
| little egret | XP_009638257.1 | E | D | D | A | T | No |
| burrowing owl | XP_026705725.1 | Q | D | A | L | T | No |
| ruff | XP_014815705.1 | Q | D | D | L | T | No |
| Apteryx mantelli | XP_013805736.1 | K | D | D | L | I | No |
| zig-zag eel | XP_026175949.1 | R | D | P | E | I | No |
| Indian glassy fish | XP_028257887.1 | T | D | P | E | I | No |
| large yellow | XP_010730146.1 | K | N | P | I | I | No |
| croaker | | | | | | | |
| Aquila chrysaetos | XP_011587755.1 | Q | D | D | L | T | No |
| canadensis | | | | | | | |
| tufted duck | XP_032058386.1 | Q | D | P | L | L | No |
| Aquila chrysaetos | XP_029855025.1 | Q | D | D | L | T | No |
| Myotis davidii | XP_006775273.1 | Q | N | P | T | I | Yes |
| prairie deer | XP_006973269.1 | Q | N | L | I | I | No |
| mouse | | | | | | | |
| yellow-throated | XP_010084373.1 | Q | D | V | L | T | No |
| sandgrouse | | | | | | | |
| tongue sole | XP_016887914.1 | K | D | P | E | I | No |
| Chinese tree | XP_006164754.1 | Q | D | T | T | E | No |
| shrew | | | | | | | |
| chuck-will’s- | XP_010169238.1 | Q | D | A | L | I | No |
| widow | | | | | | | |
| pike-perch | XP_031162227.1 | K | D | P | E | I | No |
| dingo | XP_025292925.1 | Q | D | S | T | V | No |
| Miniopterus | XP_016058453.1 | Q | N | S | S | T | Yes |
| natalensis | | | | | | | |
| Bengalese finch | XP_021388026.1 | A | D | D | P | T | No |
| denticle herring | XP_028837781.1 | T | D | P | T | N | No |
| Cichlid | XP_005724169.1 | N | D | L | E | I | No |
| Okarito brown | XP_025942946.1 | K | D | D | L | I | No |
| kiwi | | | | | | | |
| Balaenoptera | XP_028020351.1 | Q | N | L | T | L | Yes |
| acutorostrata | | | | | | | |
| scammoni | | | | | | | |
| striped catfish | XP_026803610.1 | S | D | Q | E | I | No |
| blue-crowned | XP_017667729.1 | E | D | N | L | I | No |
| manakin | | | | | | | |
| golden-collared | XP_017939494.2 | D | D | N | L | I | No |
| manakin | | | | | | | |
| Saker falcon | XP_005443093.2 | Q | D | A | L | T | No |
| Coquerel’s sifaka | XP_012494185.1 | Q | N | V | T | V | Yes |
| Anser cygnoides | XP_013039300.1 | Q | D | P | L | I | No |
| domesticus | | | | | | | |
| White-ruffed | XP_027494818.1 | E | D | N | L | I | No |
| manakin | | | | | | | |
| Wild Bactrian | XP_006194263.1 | Q | N | V | T | L | Yes |
| camel | | | | | | | |
| wolf-eel | XP_031702716.1 | N | D | T | K | I | No |
| blunt-snouted | XP_028297875.1 | T | D | L | G | I | No |
| clingfish | | | | | | | |
| Struthio camelus | XP_009667495.1 | N | D | D | L | I | No |
| australis | | | | | | | |
| Grammomys | XP_028617961.1 | K | T | P | I | I | No |
| surdaster | | | | | | | |
| pinecone | XP_029904152.1 | T | D | P | E | I | No |
| soldierfish | | | | | | | |
| Ugandan red | XP_023054821.1 | Q | N | L | T | V | Yes |
| Colobus | | | | | | | |
| Wire-tailed | XP_027593974.1 | D | D | N | L | I | No |
| manakin | | | | | | | |
| Damara mole-rat | XP_010643477.1 | Q | N | L | T | V | Yes |
| Corvus cornix | XP_010392735.2 | T | D | D | L | T | No |
| Upper Galilee | XP_008839098.1 | Q | D | L | V | I | No |
| mountains blind | | | | | | | |
| mole rat | | | | | | | |
| New Caledonian | XP_031956594.1 | T | D | D | L | T | No |
| crow | | | | | | | |
| Orycteropus afer | XP_007951028.1 | S | N | S | T | I | Yes |
| yellow catfish | XP_027024524.1 | S | D | P | E | T | No |
| Sinocyclocheilus | XP_016345325.1 | S | D | P | L | I | No |
| anshuiensis | | | | | | | |
| Paramormyrops | XP_023679669.1 | T | D | P | T | I | No |
| kingsleyae | | | | | | | |
| Yangtze finless | XP_024599894.1 | R | N | P | T | L | No |
| porpoise | | | | | | | |
| Cebus capucinus | XP_017367865.1 | Q | N | L | T | V | Yes |
| imitator | | | | | | | |
| Goodes | XP_030407881.1 | T | D | P | I | V | No |
| thornscrub | | | | | | | |
| tortoise | | | | | | | |
| Seriola lalandi | XP_023257445.1 | K | N | P | E | I | No |
| dorsalis | | | | | | | |
| Philippine tarsier | XP_008062810.1 | Q | N | S | T | I | Yes |
| Kakapo | XP_030332639.1 | E | D | A | L | T | No |
| budgerigar | XP_005151516.1 | Q | D | A | L | T | No |
| southern white | XP_004435206.1 | Q | N | V | T | V | Yes |
| rhinoceros | | | | | | | |
| Florida manatee | XP_004386381.1 | S | S | S | V | I | No |
| Enhydra lutris | XP_022374078.1 | Q | D | P | I | N | No |
| kenyoni | | | | | | | |

Variants that are predicted to reduce the virus S-protein interactions and thereby decrease S/ACE2 binding affinity include K31R, N33I, H34R, E35K, E37K, D38V, Y50F, N51S, K68E, F72V, Y83H, G326E, G352V, D355N and Q388L. Below we discuss the structural basis for the inhibitory effect on ACE2/S-protein binding for this selected set of mutations, as well as for the enhancing effect of the selected polymorphisms that were shown to increase ACE2/S-protein binding in vitro (Procko, 2020).

TABLE 3
S-protein affinity for ACE2 variants

| | ELISA | | |
|---|---|---|---|
| hACE2 | EC50, nM | | |
| variant | [ACE-2-Fc] | | |
| | S-RBD | S1 | S-trimer |
| WT | 1.01 ± 0.04 | 10.4 ± 0.05 | 0.95 ± 0.03 |
| S19P | 0.77 ± 0.08 | 4.16 ± 0.13 | 1.20 ± 0.03 |
| K26R | 0.89 ± 0.11 | 5.04 ± 0.02 | 0.62 ± 0.06 |
| K31R | 298 ± 0.64 | NB | 73 ± 0.07 |
| N33I | 2.06 ± 0.04 | 20.48 ± 0.06 | 1.60 ± 0.06 |
| H34R | 1.85 ± 0.05 | 18.82 ± 0.04 | 2.71 ± 0.03 |
| E37K | 15.8 ± 0.03 | NB | 17.6 ± 0.02 |
| A80G | 1.82 ± 0.06 | 12.84 ± 0.04 | 1.93 ± 0.03 |
| N90E | 0.66 ± 0.06 | 4.13 ± 0.03 | 0.32 ± 0.04 |
| N90T | 3.05 ± 0.03 | 11.08 ± 0.04 | 3.20 ± 0.03 |
| T92I | 0.48 ± 0.03 | 3.22 ± 0.03 | 0.47 ± 0.04 |

NB—no binding

TABLE 4
ACE2 variants comprising two or more amino acid substitutions
compared to WT human ACE2 protein for
enhanced binding to SARS-CoV-2 S-protein

| S No | Combination variants |
|---|---|
| 1 | S19P-K26R |
| 2 | S19P-N90E |
| 3 | S19P-T92I |
| 4 | K26R-N90E |
| 5 | K26R-T92I |
| 6 | S19P-K26R-N90E |
| 7 | S19P-K26R-N92I |

### Discussion

The host-virus evolutionary arms race over time leads to natural selection that alters both the host and the viral proteins allowing both to increase their fitness (Daugherty and Malik, 2012). In this context, multiple studies have analyzed and identified the origin, evolution and successful adaption of the SARS coronaviruses as human pathogens (Andersen et al., 2020; Guo et al., 2020). Viral genome sequencing and analysis have identified bats as the most likely natural host of origin for both SARS-CoV and the recent SARS-CoV-2 (Guo et al., 2020). In particular, several studies have focused on the viral S-protein RBD that interacts with its host ACE2 receptor and identified key changes between the bat CoVs and other suspected intermediary host CoVs found in the civet and pangolin (Andersen et al., 2020; Chen et al., 2020; Shang et al., 2020; Walls et al., 2020; Wrapp et al., 2020; Yan et al., 2020). These studies have identified S-protein changes that have rendered the human cells permissive to the SARS-CoV and SARS-CoV-2 infection (Chen et al., 2020; Shang et al., 2020; Walls et al., 2020; Wrapp et al., 2020; Yan et al., 2020).

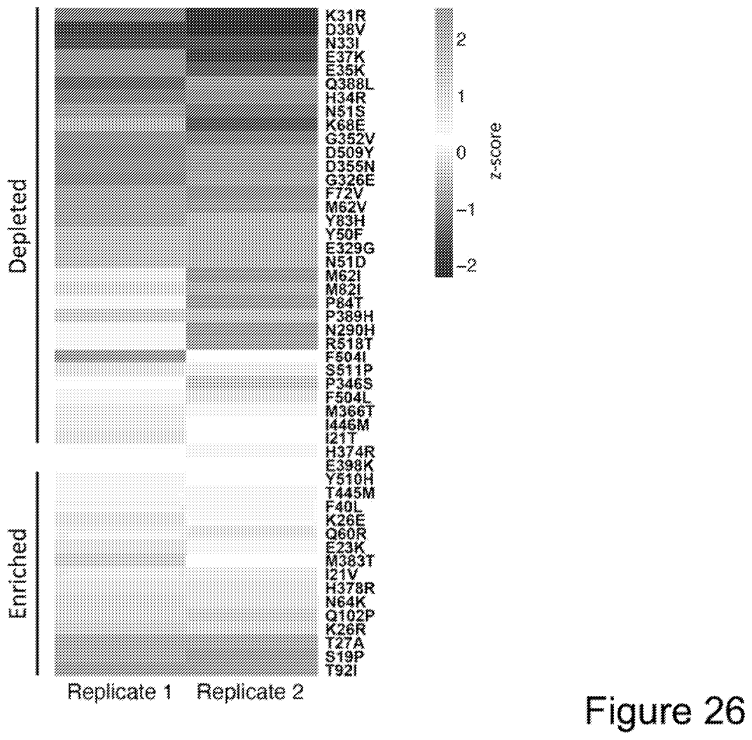

Thus far, the role of variations in human ACE2 receptor in susceptibility to both SARS CoVs had not been comprehensively examined. While a recent in silico study analyzed limited ACE2 population variation data set and concluded that these polymorphisms did not confer resistance to the virus (Cao et al., 2020a), other studies have implicated ACE2 variants in altering binding to S-protein (Benetti et al., 2020; 338 Cirulli et al., 2020; Devaux et al., 2020; Hou et al., 2020; Hussain et al., 2020). In this study, we comprehensively examined human ACE2 variation data compiled from multiple data sets and identified polymorphisms that will either likely render individuals more susceptible to the SARS-CoV-2 or protect them from the virus. Using published protein structures and data from a high-throughput functional mutagenesis screen that used deep sequencing to assess enrichment or depletion of S-protein binding to ACE2 variants (FIG. 26), we performed structural modeling to classify ACE2 variants identified in this study based on their effects on susceptibility to SARS-CoV (Chan et al., 2020b; Shang et al., 2020; Walls et al., 2020; Wrapp et al., 2020; Yan et al., 2020).

We identified several ACE2 polymorphic variants that increase ACE2/S-protein interaction including S19P, I21V, E23K, K26R, K26E, T27A, N64K, T92I, Q102P, M383T and H378R. Among these, the T92I polymorphism is part of a NxT/S consensus N-glycosylation motif (Gavel and von Heijne, 1990). The ACE2 NxT/S motif, while conserved in 96 out of 296 jawed vertebrates, it is absent or altered in several species, including the civet cat (Paguma larvata). The NxT/S motif is altered in several bat species and this includes substitution at N90, presence of a proline at position 91 or any amino acid except serine at T92, any of which will abolish the glycosylation at N90 (FIG. 1d, FIG. 3 and Table 2) (Damas et al., 2020; Demogines et al., 2012; Gavel and von Heijne, 1990; Li et al., 2005b). These ACE2 variations are expected to abolish glycosylation at N90 (Gavel and von Heijne, 1990). Another mutation that altered the NxT/S motif in human ACE2 to a civet ACE2-like sequence (90-NLTV-93 to DAK1), also expected to abolish the N-glycosylation, was shown to increase the SARS-CoV infectivity and S-protein binding (FIG. 1d) (Li et al., 2005b). Using recombinant T92I mutant ACE2 protein, we showed that it had an increased affinity for S-RBD and also found it to be more effective in blocking virus entry compared to ACE2 WT (Table 1). Further, the T92I mutant showed a strong enrichment in a sequencing-based screen for S-protein binders (Chan et al., 2020b). Thus, the T92I mutation likely renders individuals harboring this mutation more susceptible to the virus. Taken together, these observations suggest that N90 glycosylation site is critical and it could confer protection through glycan shielding. ACE2 N90 glycosylation could also determine the strength and specificity of infection by different CoV viruses.

We also show that another ACE2 residue, K26, plays an important role in controlling the susceptibility to viral infections. Our biochemical binding assays showed increased affinity of K26R ACE2 for S-protein (Table 3 and FIGS. 19-22). We also found ACE2 variants with decreased S/ACE2 binding affinity. Biochemical binding assays showed decreased affinity of two variants that we tested, K31R and E37K, indicating that these likely are protective polymorphism. Overall, we find the ACE2 population variants, that either increase or decrease susceptibility, to be rare, which is consistent with the overall low number of ACE2 receptor population level polymorphisms (mean Fst 0.0167). Also, we did not observe significant differences in ACE2 variant allele frequency among population groups. The variant alleles also did not show discernable gender distribution differences, even though ACE2 is a X-linked gene. The SARS-CoV infections and its deadly effects in humans are more recent and thus the pathogenic and protective variants have not been subject to purifying selection and therefore are predictably rare. The expression levels of ACE2 and its variants in appropriate host tissue may modulate the deleterious effect of the virus. To further understand the importance of the ACE2 variants in susceptibility, it will be important to correlate clinical outcomes with ACE2 genotypes at population scale. ACE2 K26R, predicted to increase susceptibility to SARS-CoV-2, is found in 8 women and 6 men in the UK Biobank exome sequencing dataset. Two of the 6 men tested positive for SARS-CoV-2 infection, representing a (non-significant) 2.4-fold increased odds of infection compared to those who do not carry the variants (Fisher's exact p=0.279). No other variants with predicted binding affinity were found in the UK Biobank participants with both exome sequencing data and COVID-19 test results. Genetic variation in ACE2 alone is unlikely to explain the vast variability in infection susceptibility and severity of COVID-19. While a handful of large genome-wide association studies (GWAS) of SARS-CoV-2 infection status have identified additional genetic risk factors (Ellinghaus et al., 2020; Kachuri et al., 2020), the ACE2 locus shows only weak association in these studies, possibly due to the lack of common variation in the locus. The extremes in COVID-19 clinical symptoms reported range from asymptomatic infected adult individuals to those that show acute respiratory syndrome leading to death (Cao et al., 2020b; Cascella et al., 2020; Yuen et al., 2020). This suggests a role for additional factors, including the role of innate and adaptive immunity, besides variation in ACE2 in modifying disease outcomes.

Currently, there are no approved targeted therapeutics for curing SARS-CoV-2 infection. Therefore, development of therapeutics to treat patients and mitigate the COVID-19 pandemic is urgently needed (Cascella et al., 2020; Jiang, 2020). Several small molecules and neutralizing antibodies for treatment are in development (Li and De Clercq, 2020; Zhou et al., 2020b). Soluble ACE2 and ACE2-Fc fusion protein have been proposed as decoy SARS-CoV-2 receptor therapeutic (Hofmann et al., 2004; Kruse, 2020; Lei et al., 2020). Soluble ACE2, as a therapy for pulmonary arterial hypertension, has been shown to be safe in early human clinical studies (Guignabert et al., 2018; Haschke et al., 2013). A rationally designed, catalytically inactive, human ACE2 that carries one or more of the natural variants predicted to show improved binding to SARS viral S-protein RBD that could be safely developed as a soluble protein with or without an Fc domain for treatment of COVID-19 is proposed herein.

Even though a human recombinant soluble ACE2 is in clinical trials to treat SARS-CoV-2 infection (Zoufaly et al., 2020), a catalytically-inactive soluble ACE2 might be preferred from a safety perspective, as S-protein binding enhances ACE2's carboxypeptidase activity (Lu and Sun, 2020). Additionally, as ACE2 enzymatic activity modulates multiple biological pathways (Arendse et al., 2019), a catalytically inactive form should be considered for treating SARS-CoV-2 infection, as is disclosed herein. Such a recombinant ACE2 protein can be engineered to create a pan-CoV neutralizing drug (see for example, FIG. 7C) with enhanced SARS CoV-2 virus binding mutations (see for example, FIGS. 7E, 7F, 7G, 7H, 11 and 17 as well as other enhancing mutations, singly or in combination, as disclosed herein) that that is broad and can neutralize CoVs that may emerge during future epidemics. Understanding the natural ACE2 polymorphism spectrum not only provides information on the SARS-CoV-2 susceptibility but can also be used to generate high-affinity, rationally designed soluble ACE2 receptor molecules. Such agents that carry naturally occurring polymorphism(s) will lead to no or low immunogenicity in a drug setting and can be used as a decoy receptor for treating patients.

### Example 3

### Exemplary Human ACE2 Protein Fusion Proteins and Variants

Full length human ACE2 protein encoded by human ACE2 gene is illustrated in FIG. 4. The human ACE2 protein has a signal sequence (amino acid residues 1-17, red box or darkest box), followed by an extracellular domain (amino acid residues 18-740, light blue box or an interrupted box labeled “ecd” extending to the proximal border of box labeled “tm”) comprising a peptidase domain (amino acid residues 18-617) with a HEMGH zinc binding domain (374-378, brown box or a dark box within the “ecd” box) required for peptidase activity and a collectrin domain (amino acid residues 617-740 or later portion of the 2^{nd} half of the “ecd” box), a transmembrane domain (amino acid residues 741-763, green box or box labeled “tm”), and a cytosolic domain (amino acid residues 762-805, gray box or box labeled “cd” at C-terminus). The amino acid sequence of the human ACE2 protein is provided below (UniProtKB ID: Q9BYF1-1; SEQ ID NO: 1) and serves as a reference sequence for defining ACE2 variants (see FIG. 1C and Table 1 for human ACE2 allelic variants).

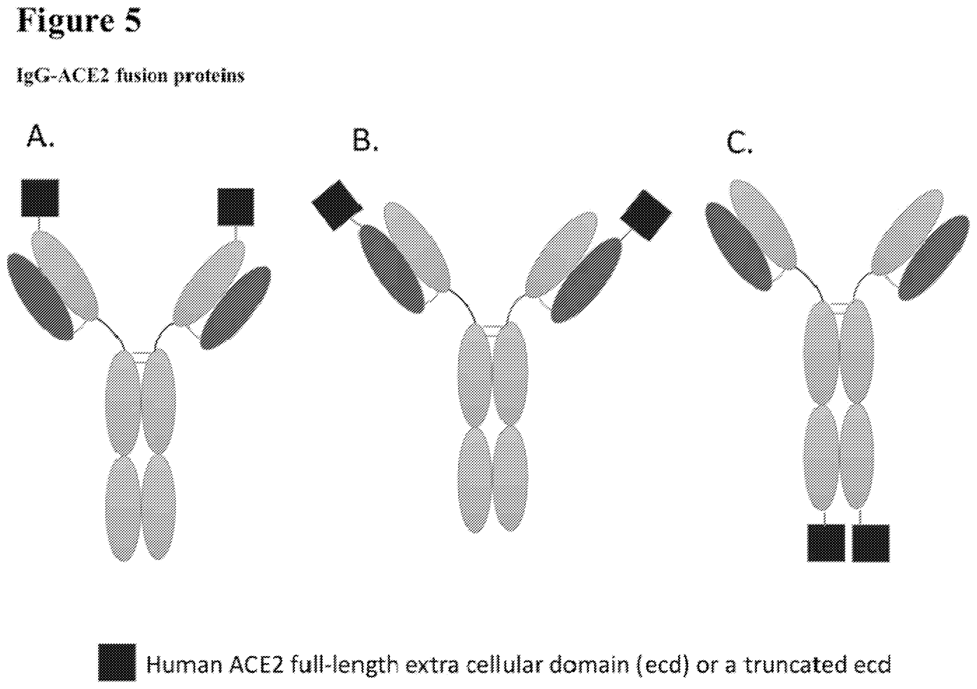

Exemplary IgG-ACE2 fusion proteins comprising a human ACE2 full-length extracellular domain (ecd) or a truncated ACE2 ecd and an IgG are shown in FIG. 5. Human ACE2 ecd or its fragment may be fused to the N-terminus of an immunoglobulin light chain or heavy chain, or alternatively, to the C-terminus of an immunoglobulin heavy chain. A signal sequence may be present or be lacking from the ACE2 ecd. The ACE2 ecd or its fragment may contain amino acid substitution(s) to increase binding or binding affinity of ACE2 for SARS-CoV-2 virus or SARS-CoV-2 S-protein (SARS-CoV-2-S), as described in the instant invention. IgG may be replaced with IgM, IgD, IgE or IgA. The fusion protein may be modified so as increase its half-life or bioavailability when used in situ or in vivo.

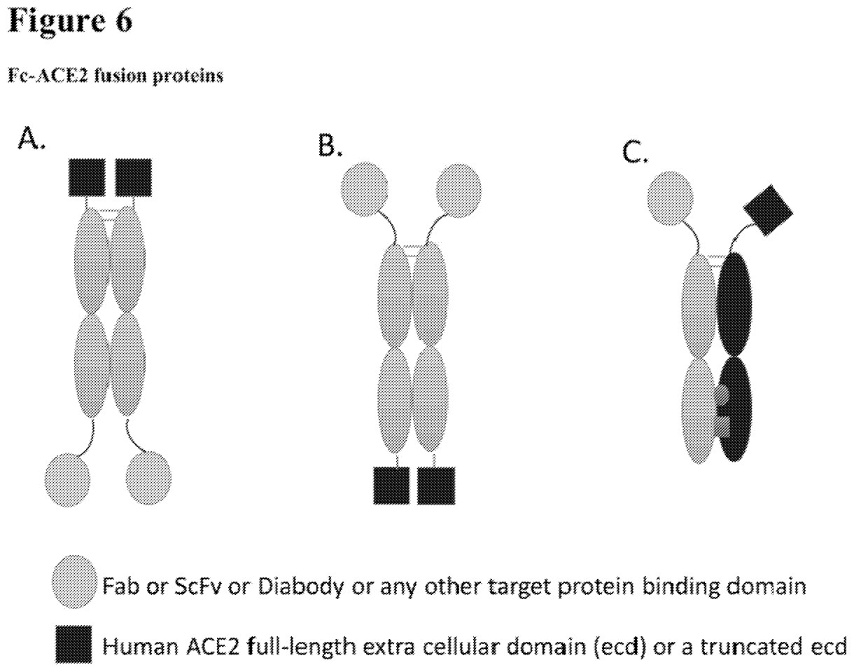

FIG. 6 provides exemplary fusion protein comprising a human ACE2 ecd or its fragment or a variant thereof, an immunoglobulin heavy chain fragment, Fc, and a Fab, scFv, diabody or any other target protein binding domain. In an embodiment, the Fc fragment may be fused at its N-terminus with a human ACE2 ecd or its fragment or a variant thereof and at its C-terminus with a Fab, scFv, diabody or any other target protein binding domain. The Fc fragment forms a homodimer stabilized by intermolecular disulfide bonds in their respective hinge regions. In another embodiment, the Fc fragment may be fused at its N-terminus with a Fab, scFv, diabody or any other target protein binding domain and at its C-terminus with a human ACE2 ecd or its fragment or a variant thereof. Similarly, the Fc fragment forms a homodimer stabilized by intermolecular disulfide bonds in their respective hinge regions. In a separate embodiment, the Fc-ACE2 fusion protein may be a heterodimer of two different heavy chains comprising a first polypeptide comprising a Fab, scFv, diabody or any other target protein binding domain fused to the N-terminus of an immunoglobulin heavy chain fragment, Fc, and a second polypeptide comprising a human ACE2 ecd or its fragment or a variant thereof fused to the N-terminus of an immunoglobulin heavy chain fragment, Fc. Heterodimer formation is mediated through the Fc fragment. To favor heterodimer formation over homodimer formation, each polypeptide chain is engineered within the Fc portion, preferably corresponding to the immunoglobulin heavy chain C_{H}3 constant region, using a “knob-in-hole” protein design, wherein a “knob” or “hole” present or introduced by mutation into the first polypeptide fits into a “hole” or “knob” present or introduced into the second polypeptide so as to favor heterodimer formation over homodimer formation. The heterodimer, so formed, is further stabilized by intermolecular disulfide bond between the hinge regions of the two polypeptides in the heterodimer. A signal sequence may be present or be lacking from the ACE2 ecd. In an embodiment, the variant of the ACE2 ecd or its fragment may contain amino acid substitution(s) to increase binding or binding affinity of ACE2 for SARS-CoV-2 virus or SARS-CoV-2 S-protein, as described in the instant invention. In an embodiment, the fusion protein may be modified so as increase its half-life or bioavailability when used in situ or in vivo.

FIG. 7 illustrates exemplary hACE2 therapeutic variants. FIG. 7A Sequence: Fusion protein (i.e., SARS-CoV-2 binding protein complex) comprising a human ACE2 extracellular domain comprising amino acid residues 1-740 (signal peptide sequence and extracellular domain (ecd) with both peptidase and collectrin domains) of a human ACE2 protein or a fragment thereof and an immunoglobulin Fc domain comprising a hinge region for formation of homodimer and D265A and N297G mutations to eliminate antibody effector functions or a portion thereof, and wherein the SARS-CoV-2 binding protein complex binds SARS-CoV-2 virus or SARS-CoV-2 S-protein. FIG. 7B Sequence: Fusion protein (i.e., SARS-CoV-2 binding protein complex) comprising a human ACE2 extracellular domain comprising amino acid residues 1-615 (signal peptide sequence and peptidase domain of the ecd) or a fragment thereof and an immunoglobulin Fc domain comprising a hinge region for formation of homodimer or a fragment thereof and D265A and N297G mutations to eliminate antibody effector functions or a portion thereof, and wherein the SARS-CoV-2 binding protein complex binds SARS-CoV-2 virus or SARS-CoV-2 S-protein. FIG. 7C Sequence: Fusion protein (i.e., SARS-CoV-2 binding protein complex) comprising a human ACE2 extracellular domain comprising amino acid residues 1-615 (signal peptide sequence and peptidase domain of the ecd) of human ACE2 protein and H374N and H378N mutations to inactivate peptidase activity, or a fragment thereof, and an immunoglobulin Fc domain comprising a hinge region for formation of homodimer and D265A and N297G mutations to eliminate antibody effector functions, or a portion thereof, and wherein the SARS-CoV-2 binding protein complex binds SARS-CoV-2 virus or SARS-CoV-2 S-protein. FIG. 7D is a schematic diagram of ACE2-ecd-Fc-DANG fusion protein homodimer (i.e., SARS-CoV-2 binding protein complex) of FIG. 7C with intermolecular disulfide bonds at hinge region of Fc fragment and inactivated peptidase activity of ACE2. FIG. 7E is a schematic diagram of a fusion protein (i.e., SARS-CoV-2 binding protein complex) comprising an ACE2 extracellular domain (amino acid residues 18-615 of human ACE2 protein) that comprises one or more mutations in the ACE2 extracellular domain that enhance binding to SARS-CoV-2 virus or SARS-CoV2-S protein (SARS-CoV-2 S-protein) and H374N and H378N mutations eliminating peptidase activity of the extracellular domain, or a fragment thereof, and immunoglobulin Fc fragment comprising a hinge region for formation of homodimer, or a portion thereof, and wherein the SARS-CoV-2 binding protein complex binds SARS-CoV-2 virus or SARS-CoV-2 S-protein. ACE2 mutations that enhances the binding to SARS-CoV-2 virus or SARS-CoV-2 S-protein are any of S19P, I21V, E23K, K26E, K26R, T27A, N33I, F40L, N64A, A80G, N90E, N90T, T92I, Q102P, H378R, M383T and T445M or a combination thereof. In an embodiment, the ACE2 therapeutic protein is a fusion protein (i.e., SARS-CoV-2 binding protein complex) comprising a human ACE2 extracellular domain or a fragment thereof and an immunoglobulin Fc fragment or portion thereof, wherein the ACE2 extracellular domain comprises one or more mutations selected from the group consisting of S19P, I21V, E23K, K26E, K26R, T27A, N33I, F40L, N64A, A80G, N90E, N90T, T92I, Q102P, H378R, M383T and T445M or a combination thereof, wherein the fusion protein binds SARS-CoV-2 virus or SARS-CoV-2 S-protein. In a preferred embodiment, the fusion protein comprises an ACE2 extracellular domain or its fragment comprising two or mom mutations selected from the group consisting of S19P-K26R, S19P-N90E, S19P-T92I, K26R-N90E, K26R-T92I, S19P-K26R-N90E and S19P-K26R-N92I and an immunoglobulin Fc fragment, preferably with H374N and H378N mutations. FIG. 7F is a SARS-CoV-2 binding protein complex (Fc-DANG complex) comprising a human ACE2 extracellular domain comprising amino acid residues 1-615 of the human ACE2 protein and additionally comprising T92I mutations that results in improved binding to SARS-CoV-2 virus or SARS-CoV-2 S-protein and H374N-H378N mutations which results in an inactive protease domain, or a fragment thereof, and an IgG Fc fragment comprising amino acid residues 221-447 comprising a hinge region for formation of homodimer and D265A and N297G mutations which eliminate immunoglobulin effector function, or a portion thereof, and wherein the SARS-CoV-2 binding protein complex binds SARS-CoV-2 virus or SARS-CoV-2 S-protein. FIG. 7G is a SARS-CoV-2 binding protein complex (Fc-DANG complex) comprising a human ACE2 extracellular domain comprising amino acid residues 1-615 of human ACE2 protein and additionally comprising A80G and T92I mutations that result in improved binding to SARS-CoV-2 virus or SARS-CoV-2 S-protein and H374N-H378N mutations which results in an inactive protease domain, or a fragment thereof, and an IgG Fc fragment comprising amino acid residues 221-447 comprising a hinge region for formation of homodimer and D265A and N297G mutations which eliminate immunoglobulin effector function, or a portion thereof, and wherein the SARS-CoV-2 binding protein complex binds SARS-CoV-2 virus or SARS-CoV-2 S-protein. FIG. 7H is a Fc-DANG complex that contains combined ACE2 mutations that results in improved CoV2-S binding (N33I-A80G) with T92I and inactive protease domain (H374N-H378N). FIG. 7H is a SARS-CoV-2 binding protein complex (Fc-DANG complex) comprising a human ACE2 extracellular domain comprising amino acid residues 1-615 of human ACE2 protein and additionally comprising N33I, A80G and T92I mutations that result in improved binding to SARS-CoV-2 virus or SARS-CoV-2 S-protein and H374N-H378N mutations which results in an inactive protease domain, or a fragment thereof, and an IgG Fc fragment comprising amino acid residues 221-447 comprising a hinge region for formation of homodimer and D265A and N297G mutations which eliminate immunoglobulin effector function, or a portion thereof, and wherein the SARS-CoV-2 binding protein complex binds SARS-CoV-2 virus or SARS-CoV-2 S-protein. Other SARS-CoV-2 binding protein complex (Fc-DANG complex) contemplated are SARS-CoV-2 binding protein complex comprising a human ACE2 fragment comprising amino acid residues 1-615 and additionally comprising N33I, A80G and T92I mutations that results in improved binding to SARS-CoV-2 virus or SARS-CoV-2 S-protein and H374N-H378N mutations which results in an inactive protease domain, or a fragment thereof, and an IgG Fc fragment comprising amino acid residues 221-447 comprising a hinge region for formation of homodimer and D265A and N297G mutations which eliminate immunoglobulin effector function, or a portion thereof, and wherein the SARS-CoV-2 binding protein complex binds SARS-CoV-2 virus or SARS-CoV-2 S-protein.

FIG. 8 is a schematic diagram and amino acid sequence of an HHB (helix2-helix1-beta turn), a novel truncated ACE2 therapeutic agent comprising a helix forming peptide 2 (amino acid residues 55-83 of human ACE2 protein (SEQ ID NO:6) or variant or equivalent; helix 2), another helix forming peptide 1 (amino acid residues 22-52 of human ACE2 protein (SEQ ID NO: 7) or variant or equivalent; helix 1) and a beta turn peptide (amino acid residues 348-357 (SEQ ID NO: 8) or variant or equivalent; beta turn) and an immunoglobulin Fc fragment (amino acid residues 221-447) or a portion thereof. SARS-CoV2-S interactions domains in the ACE2ecd are covalently linked by GG linker (no shading) between helix 2 forming peptide anteriorly and helix 1 forming peptide posteriorly and GGGGSGG linker between helix 1 forming peptide and beta turn peptide, which is directly joined to the Fc fragment. The IgG-Fc domain has D265A and N297G mutations to eliminate antibody effector functions. The first 19 amino acids (shaded dark) followed by a glycine residue as a linker at the N-terminus of the synthetic protein is a signal peptide sequence which is normally process out of the mature protein following in vivo expression. Variants of SEQ ID NO: 6 can be any of A80G, M82I and Y83H or a combination thereof. Variants of SEQ ID NO: 7 can be any of K26R, K26E, T27A, K31R, N33I, H34R, E35K, E35D, E37K and D38V or a combination thereof. In an embodiment, variants of SEQ ID NO: 10 comprises improved binding of SARS-CoV-2 virus of SARS-CoV-2 S-protein by HHB SARS-CoV-2 binding protein complex, wherein the combination is selected from the group consisting of K26R-N33I, K26R-H34R, K26E-N33I, K26E-H34R, N33I-H34R, K26R-N33I-H34R and K26E-N33I-H34R and optionally one or more additional substitutions selected from the group consisting of E35K, E35D, E37K and D38V, and wherein the improved binding is higher binding affinity of the variant over wild-type HHB.

FIG. 9 shows the amino acid sequence of a minHHB, a novel truncated ACE2 therapeutic agent. In minHHB, the helix 1 and 2 and beta turn peptides are further truncated compared to HHB. Helix 1 is truncated to SEQ ID NO: 10 and helix 2 is truncated to SEQ ID NO: 9. Beta turn is truncated to SEQ ID NO: 11. Similarly, minHHB is a novel truncated ACE2 therapeutic agent comprising a truncated helix forming peptide 2 (amino acid residues 65-83 of human ACE2 protein (SEQ ID NO:9) or variant or equivalent), another truncated helix forming peptide 1 (amino acid residues 22-44 of human ACE2 protein (SEQ ID NO: 10) or variant or equivalent) and a beta turn peptide (amino acid residues 348-357 (SEQ ID NO: 8) or variant or equivalent) and an immunoglobulin Fc fragment (amino acid residues 221-447) or a portion thereof. SARS-CoV2-S interactions domains in the ACE2ecd are covalently linked by GG linker (no shading) between helix 2 forming peptide anteriorly and helix 1 forming peptide posteriorly and GGGGSGG linker between helix 1 forming peptide and beta turn peptide, which is directly joined to the Fc fragment. The IgG-Fc domain has D265A and N297G mutations to eliminate antibody effector functions. The first 19 amino acids (shaded dark) followed by a glycine residue as a linker at the N-terminus of the synthetic protein is a signal peptide sequence which is normally process out of the mature protein following in vivo expression. Variants of SEQ ID NO: 9 can be any of A80G, M82I and Y83H or a combination thereof. Variants of SEQ ID NO: 10 can be any of K26R, K26E, T27A, K31R, N33I, H34R, E35K, E35D, E37K and D38V or a combination thereof. In an embodiment, variants of SEQ ID NO: 10 comprises improved binding of SARS-CoV-2 virus of SARS-CoV-2 S-protein by minHHB SARS-CoV-2 binding protein complex, wherein the combination is selected from the group consisting of K26R-N33I, K26R-H34R, K26E-N33I, K26E-H34R, N33I-H34R, K26R-N33I-H34R and K26E-N33I-H34R and optionally one or more additional substitutions selected from the group consisting of E35K, E35D, E37K and D38V, and wherein the improved binding is higher binding affinity of the variant over wild-type minHHB.

FIG. 10 is a schematic diagram of an HB (helix1-beta turn), a novel truncated ACE2 therapeutic agent including its sequence. This HB SARS-CoV-2 binding protein complex comprises a truncated helix forming peptide 1 (amino acid residues 22-44 of human ACE2 protein (SEQ ID NO: 10) or variant or equivalent) and a beta turn peptide (amino acid residues 348-357 (SEQ 1W NO: 8) or variant or equivalent) and an immunoglobulin Fc fragment (amino acid residues 221-447) or a portion thereof. SARS-CoV2-S interactions domains in the ACE2ecd are covalently linked by a single amino acid linker, glycine (no shading), anteriorly to a 19 amino acid signal peptide sequence (shaded dark) at the N-terminus of the synthetic protein and posteriorly to the beta turn peptide (also shaded dark), which in turn is directly joined to the Fc fragment at the C-terminus. The IgG-Fc domain has D265A and N297G mutations to eliminate antibody effector functions. The signal peptide sequence is normally cleaved off of the mature protein following in vivo expression. Variants of SEQ ID NO: 10 can be any of K26R, K26E, T27A, K31R, N33I, H34R, E35K, E35D, E37K and D38V or a combination thereof. In an embodiment, variants of SEQ ID NO: 10 comprises improved binding of SARS-CoV-2 virus of SARS-CoV-2 S-protein by minHHB SARS-CoV-2 binding protein complex, wherein the combination is selected from the group consisting of K26R-N33I, K26R-H34R, K26E-N33I, K26E-H34R, N33I-H34R, K26R-N33I-H34R and K26E-N33I-H34R and optionally one or more additional substitutions selected from the group consisting of E35K, E35D, E37K and D38V, and wherein the improved binding is higher binding affinity of the variant over wild-type HB.

FIG. 11 is a schematic diagram of an ACE2ecd-Fc-scFv, a bi-specific fusion protein. The SARS-CoV2-S interaction domains in the ACE2ecd are shown in color. ACE2ecd has protease function defective mutations of H374N and H378N. IgG-Fc domain has D265A and N297G mutations to eliminate antibody effector functions. The sequence is the same as shown in FIG. 7C except that this embodiment contains a C-terminal fusion of a select scFv (or a Diabody) of an anti-SARS-CoV2-S antibody (for example an ACE2 non-competing CR3022 scFv antibody fragment or it can be any ACE2 non-competing SARS-CoV2-S antibody or antibody fragment). The sequence is of an ACE2ecd-T92I-H374N-H378N-Fc (DANG)-CR3022scFv. In this embodiment, the ACE2ecd (1-615aa) contains H374N-H378N mutations and ACE2ecd is recombinantly fused to a human Fc (D265A-N297G) and the CR3022scFv is fused to C-terminus of the Fc. There are additional enhanced virus binding mutations (such as N33I or A80G or both as described in FIGS. 7G and 7H) in this embodiment. Other embodiments are any of the enhanced virus binding mutations or combination thereof described in this instant invention.

FIG. 12 is a schematic diagram that shows a bi-specific knob-hole format ACE2ecd-anti-SARS-CoV2-S antibody. Additionally, in FIG. 12, the SARS-CoV2-S interaction domains in the ACE2ecd are represented in color. ACE2ecd has protease function defective mutations of H374N and H378N. IgG-Fc domain has D265A and N297G mutations to eliminate antibody effector functions. An ACE2ecd-Fc fusion protein embodiment may have the same sequence as shown in FIG. 7C except that the ACE2ecd-Fc fusion protein has two arms with different heavy chains and a light chain. An ACE2ecd-Fc fusion protein may be paired with a select diabody or scFv of anti-SARS-CoV2-S antibody (for example an ACE2 non-competing CR3022 antibody or it can be any ACE2 non-competing SARS-CoV2-S antibody).

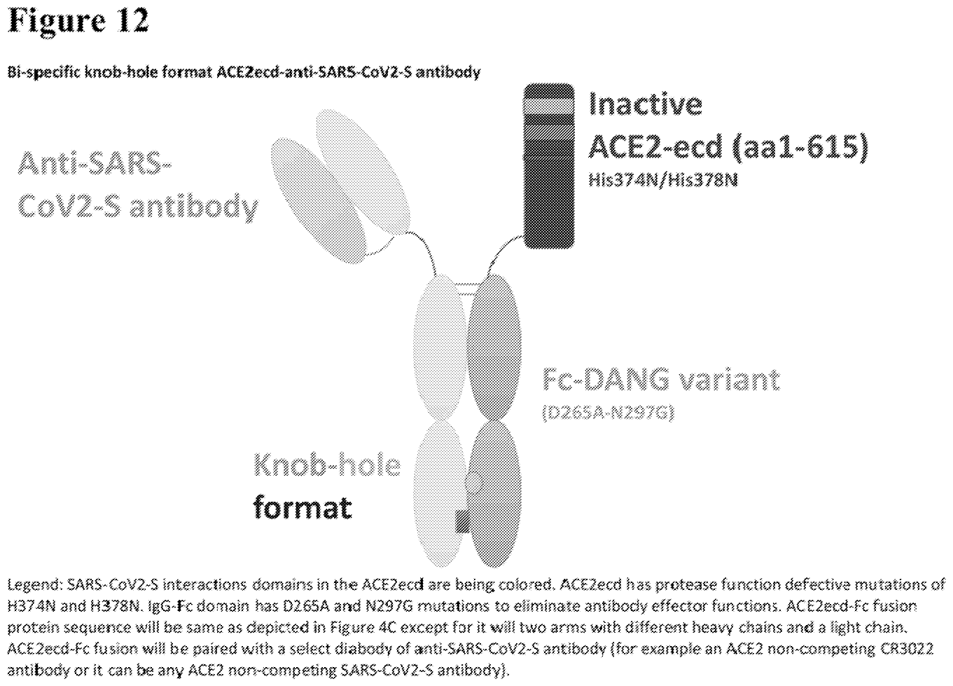

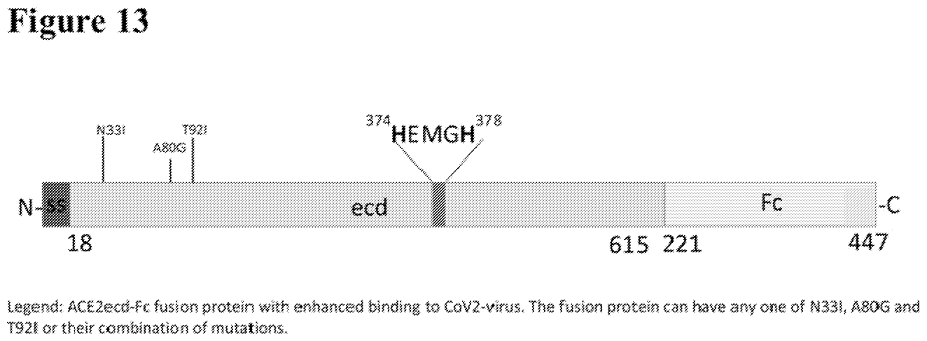

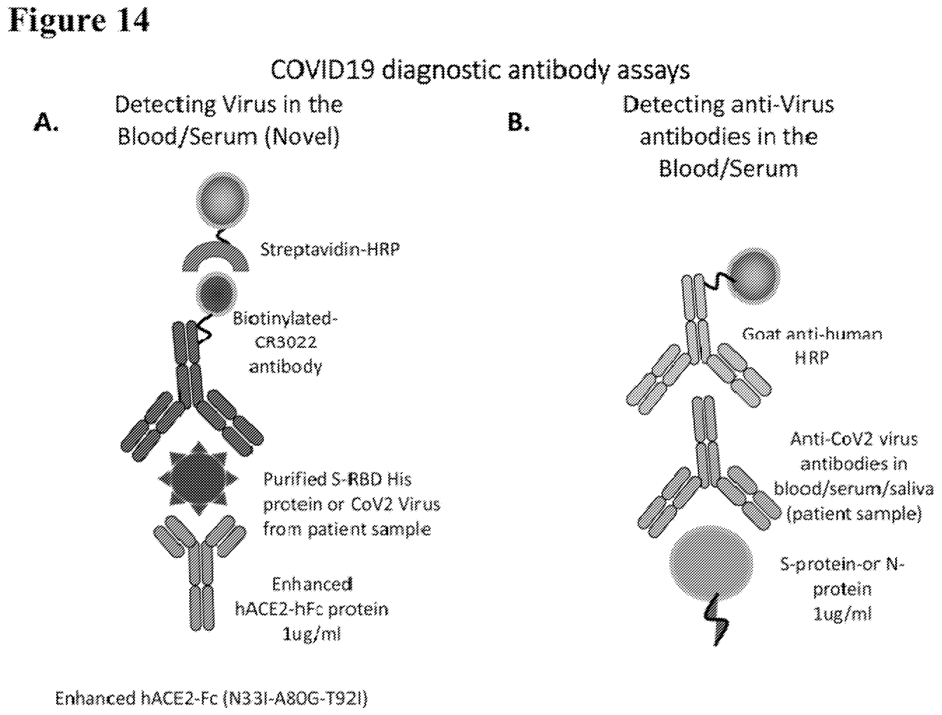

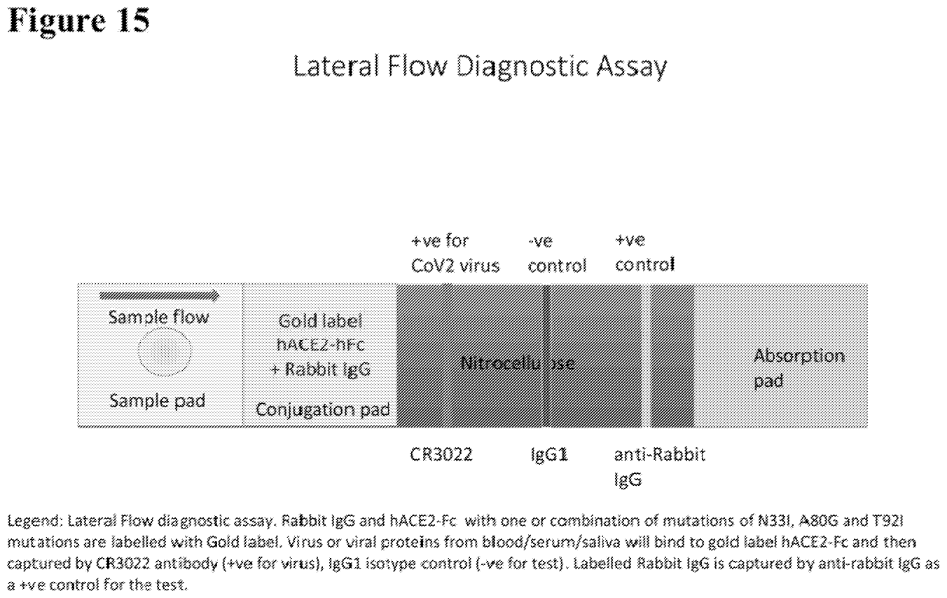

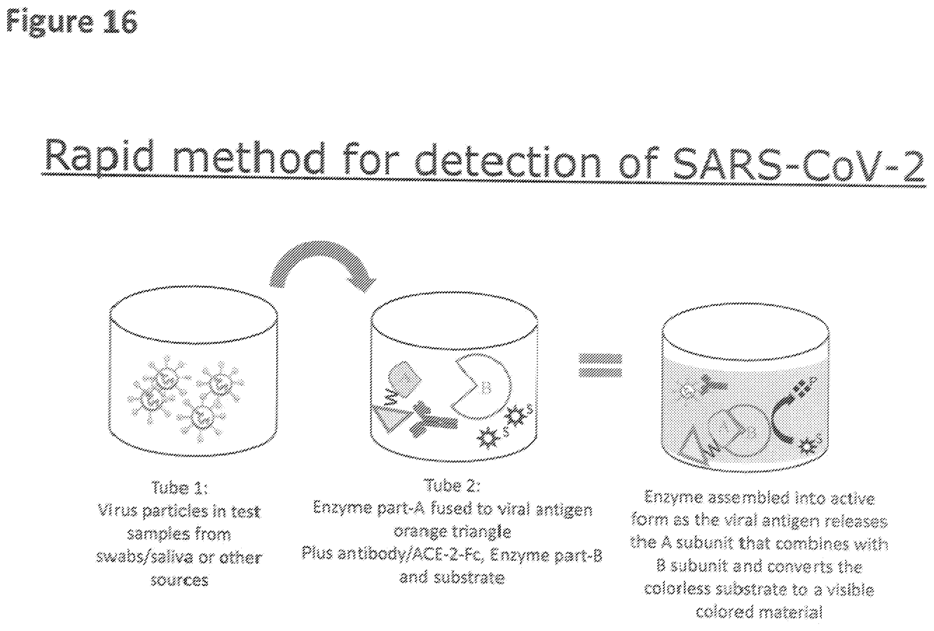

FIG. 13 shows an ACE2ecd-Fc fusion protein with enhanced binding to CoV2-virus. The fusion protein can have any one of N33I, A80G and T92I or their combination of mutations, e.g., those described herein. In an embodiment, the ACE2ecd-Fc fusion protein further comprises H374N and/or H378N mutation in the ACE2 ecd. In a separate embodiment, the ACE2ecd-Fc fusion protein further comprises D265A and/or N297G mutation in the Fc fragment. In another embodiment, the ACE2ecd-Fc fusion protein comprises H374N and/or H378N mutation in the ACE2 ecd and D265A and/or N297G mutation in the Fc fragment. In addition to the N33I, A80G and T92I or their combination of mutations, the ACE2ecd-Fc fusion protein can, in an embodiment, have one or more mutations that enhanced binding to SARS-CoV-2 virus or SARS-CoV-2 S-protein selected from the group consisting of S19P, I21V, E23K, K26E, K26R, T27A, N33I, H34R, F40L, N64K, A80G, N90E, N90I, N90T, T92I, Q102P, H378R, M383T and T445M or a combination thereof. In another embodiment, the ACE2ecd-Fc fusion protein comprises one or more mutations that enhanced binding to SARS-CoV-2 virus or SARS-CoV-2 S-protein selected from any of mutation listed as enhancing in FIG. 18, enriched in FIG. 26, having an EC50 value less than WT in Table 3, and alleles indicated by black lines in FIG. 1 or a combination thereof, so long as the selected mutation increases binding affinity of the ACE2ecd-Fc fusion protein to SARS-CoV-2 virus or SARS-CoV-2 S-protein. In a preferred embodiment, the ACE2ecd-Fc fusion protein comprises one or more mutations selected from the group consisting of S19P, K26R, N33I, H34R, A80G, N90E, N90T and T92I or a combination thereof, wherein the mutation enhanced binding of ACE2ecd-Fc fusion protein to SARS-CoV-2 virus or SARS-CoV-2 S-protein. In a more preferred embodiment, the ACE2ecd-Fc fusion protein comprises two or more mutations selected from the group consisting of S19P-K26R, S19P-N90E, S19P-T92I, K26R-N90E, K26R-T92I, S19-K26-N90 and S19-K26-T92 or a combination thereof, wherein the mutation enhanced binding of ACE2ecd-Fc fusion protein to SARS-CoV-2 virus or SARS-CoV-2 S-protein and optionally one or more additional mutations selected from the group consisting of E35K, E35D, E37K and D38V, and wherein the mutations so selected enhanced binding of ACE2ecd-Fc fusion protein to SARS-CoV-2 virus or SARS-CoV-2 S-protein.

FIG. 17 shows the amino acid sequences for bi-specific scFv's designated ACE2ecd(1-615)-(T92I)-H374N-H378N-Fc-(DANG)-3B11scFv and DPP4ecd(39-766)-S630A-Fc-(DANG)-CR3022scFv. ACE2ecd(1-615)-(T92)-H374N-H378N-Fc-(DANG)-3B1I scFv comprises ACE2 extracellular domain (amino acid residues 1-615) with enhanced SARS-CoV-2 virus or SARS-CoV-2 S-protein binding mutation(s) (e.g., T92I) and lacking peptidase activity (e.g., H374N and H378N mutations), IgG Fc fragment (amino acid residues 221-447) lacking Fc effector function (e.g., D265A and N297G mutations), and 3B11 scFv, wherein the ACE2 ecd is N-terminal and is covalently linked to Fc which in turn is covalently linked to 3311 scFv at the C-terminus of the fusion protein. DPP4ecd(39-766)-S630A-Fc-(DANG)-CR3022scFv comprises DPP4 (UniProtKB: P27487.1) extracellular domain (amino acid 39-766) comprising S630A mutation, IgG Fc fragment (amino acid residues 221-447) lacking Fc effector function (e.g., D265A and N297G mutations), and CR3022 scFv, wherein the DPP4 extracellular domain is N-terminal and is covalently linked to Fc which in turn is covalently linked to CR3022 scFv at the C-terminus of the fusion protein; wherein DPP4 extracellular domain is a fragment of Dipeptiyl peptidase-4 (UniProtKB: P27487.1) and wherein the CR3022 scFv binds to RBD of SARS-CoV-2 without blocking the binding of RBD of SARS-CoV-2 to ACE2 (PDB: 6W41). In an embodiment, bi-specific scFv's designated ACE2ecd(1-615)-(T92I)-H374N-H378N-Fc-(DANG)-3B11scFv and/or DPP4ecd(39-766)-S630A-Fc-(DANG)-CR3022scFv are used to treat a subject infected with SARS-CoV-2 virus.

### REFERENCES

Albers, P. K., and McVean, G. (2020). Dating genomic variants and shared ancestry in population-scale sequencing data. PLoS Biol 18, e3000586.
Andersen, K. G., Rambaut, A., Lipkin, W. I., Holmes, E. C., and Garry, R. F. (2020). The proximal origin of SARS-CoV-2. Nature Medicine 17 Mar. 2020.
Arendse, L. B., Danser, A. H. J., Poglitsch, M., Touyz, R. M., Burnett, J. C., Jr., Llorens-Cortes, C., Ehlers, M. R., and Sturrock, E. D. (2019). Novel Therapeutic Approaches Targeting the Renin-Angiotensin System and Associated Peptides in Hypertension and Heart Failure. Pharmacol Rev 71, 539-570.
Belouzard, S., Chu, V. C., and Whittaker, G. R. (2009). Activation of the SARS coronavirus spike protein via sequential proteolytic cleavage at two distinct sites. Proc Natl Acad Sci USA 106, 5871-5876.
Benetti, E., Tita, R., Spiga, O., Ciolfi, A., Birolo, G., Bruselles, A., Doddato, G., Giliberti, A., Marconi, C., Musacchia, F., et al. (2020). ACE2 gene variants may underlie interindividual variability and susceptibility to COVID-19 in the Italian population. Eur J Hum Genet.
Bergstrom, A., McCarthy, S. A., Hui, R., Almarri, M. A., Ayub, Q., Danecek, P., Chen, Y., Felkel, S., Hallast, P., Kamm, J., et al. (2020). Insights into human genetic variation and population history from 929 diverse genomes. Science 367, doi: 10.1126/science.aay5012.
Cao, Y., Li, L., Feng, Z., Wan, S., Huang, P., Sun, X., Wen, F., Huang, X., Ning, G., and Wang, W. (2020a). Comparative genetic analysis of the novel coronavirus (2019-nCoV/SARS-CoV-2) receptor ACE2 in different populations. Cell Discov 6, 11.
Cao, Y., Liu, X., Xiong, L., and Cai, K. (2020b). Imaging and Clinical Features of Patients With 2019 Novel Coronavirus SARS-CoV-2: A systematic review and meta-analysis. J Med Virol.
Cascella, M., Rajnik, M., Cuomo, A., Dulebohn, S. C., and Di Napoli, R. (2020). Features, Evaluation and Treatment Coronavirus (COVID-19). In StatPearls, (Treasure Island (FL)).
Chan, J. F., Yuan, S., Kok, K. H., To, K. K., Chu, H., Yang, J., Xing, F., Liu, J., Yip, C. C., Poon, R. W., et al. (2020). A familial cluster of pneumonia associated with the 2019 novel coronavirus indicating person-to-person transmission: a study of a family cluster. Lancet 395, 514-523.
Chan, K. K., Dorosky, D., Sharma, P., Abbasi, S. A., Dye, J. M., Kranz, D. M., Herbert, A. S., and Procko, E. (2020b). Engineering human ACE2 to optimize binding to the spike protein of SARS coronavirus 2. Science.
Chen, Y., Guo, Y., Pan, Y., and Zhao, Z. J. (2020). Structure analysis of the receptor binding of 2019-nCoV. Biochem Biophys Res Commun, doi: 10.1016/j.bbrc.2020.1002.1071.
Cirulli, E. T., Riffle, S., Bolze, A., and Washington, N. L. (2020). Revealing variants in SARS-CoV-2 interaction domain of ACE2 and loss of function intolerance through analysis of &gt;200,000 exomes. bioRxiv, 2020.2004.2007.030544.
Damas, J., Hughes, G. M., Keough, K. C., Painter, C. A., Persky, N. S., Corbo, M., Hiller, M., Koepfli, K. P., Pfenning, A. R., Zhao, H., et al. (2020). Broad host range of SARS-CoV-2 predicted by comparative and structural analysis of ACE2 in vertebrates. Proc Natl Acad Sci USA 117, 22311-22322.
Daugherty, M. D., and Malik, H. S. (2012). Rules of engagement: molecular insights from host-virus arms races. Annu Rev Genet 46, 677-700.
Demogines, A., Farzan, M., and Sawyer, S. L. (2012). Evidence for ACE2-utilizing coronaviruses (CoVs) related to severe acute respiratory syndrome CoV in bats. J Virol 86, 6350-6353.
Devaux, C. A., Rolain, J. M., and Raoult, D. (2020). ACE2 receptor polymorphism: Susceptibility to SARS-CoV-2, hypertension, multi-organ failure, and COVID-19 disease outcome. J Microbiol Immunol Infect 53, 425-435.
Dewey, F. E., Murray, M. F., Overton, J. D., Habegger, L., Leader, J. B., Fetterolf, S. N., O'Dushlaine, C., Van Hout, C. V., Staples, J., Gonzaga-Jauregui, C., et al. (2016). Distribution and clinical impact of functional variants in 50,726 whole-exome sequences from the DiscovEHR study. Science 354, doi: 10.1126/science.aaff814.
Ellinghaus, D., Degenhardt, F., Bujanda, L., Buti, M., Albillos, A., Invernizzi, P., Fernandez, J., Prati, D., Baselli, G., Asselta, R., et al. (2020). Genomewide Association Study of Severe Covid-19 with Respiratory Failure. N Engl J Med.
Fraser, A., Macdonald-Wallis, C., Tilling, K., Boyd, A., Golding, J., Davey Smith, G., Henderson, J., Macleod, J., Molloy, L., Ness, A., et al. (2013). Cohort Profile: the Avon Longitudinal Study of Parents and Children: ALSPAC mothers cohort. Int J Epidemiol 42, 97-110.
Gavel, Y., and von Heijne, G. (1990). Sequence differences between glycosylated and non-glycosylated Asn-X-Thr/Ser acceptor sites: implications for protein engineering. Protein Eng 3, 433-442.
GenomeAsia, K. C. (2019). The GenomeAsia 100K Project enables genetic discoveries across Asia. Nature 576, 106-111.
Genomes Project, C., Auton, A., Brooks, L. D., Durbin, R. M., Garrison, E. P., Kang, H. M., Korbel, J. O., Marchini, J. L., McCarthy, S., McVean, G. A., and Abecasis, G. R. (2015). A global reference for human genetic variation. Nature 526, 68-74.
Graham, R. L., and Baric, R. S. (2010). Recombination, reservoirs, and the modular spike: mechanisms of coronavirus cross-species transmission. J Virol 84, 3134-3146.
Guignabert, C., de Man, F., and Lombes, M. (2018). ACE2 as therapy for pulmonary arterial hypertension: the good outweighs the bad. Eur Respir J 51, doi: 10.1183/13993003.13900848-13992018.
Guo, Y. R., Cao, Q. D., Hong, Z. S., Tan, Y. Y., Chen, S. D., Jin, H. J., Tan, K. S., Wang, D. Y., and Yan, Y. (2020). The origin, transmission and clinical therapies on coronavirus disease 2019 (COVID-19) outbreak—an update on the status. Mil Med Res 7, 11.
Haschke, M., Schuster, M., Poglitsch, M., Loibner, H., Salzberg, M., Bruggisser, M., Penninger, J., and Krahenbuhl, S. (2013). Pharmacokinetics and pharmacodynamics of recombinant human angiotensin-converting enzyme 2 in healthy human subjects. Clin Pharmacokinet 52, 783-792.
Hoffmann, M., Kleine-Weber, H., Schroeder, S., Kruger, N., Herrler, T., Erichsen, S., Schiergens, T. S., Herrier, G., Wu, N. H., Nitsche, A., et al. (2020). SARS-CoV-2 Cell Entry Depends on ACE2 and TMPRSS2 and Is Blocked by a Clinically Proven Protease Inhibitor. Cell, doi.org/10.1016rj.cell.2020.1002.1052.
Hofmann, H., Geier, M., Marzi, A., Krumbiegel, M., Peipp, M., Fey, G. H., Gramberg, T., and Pohlmann, S. (2004). Susceptibility to SARS coronavirus S protein-driven infection correlates with expression of angiotensin converting enzyme 2 and infection can be blocked by soluble receptor. Biochem Biophys Res Commun 319, 1216-1221.
Holmes, K. V. (2003). SARS-associated coronavirus. N Engl J Med 348, 1948-1951.
Hou, Y., Zhao, J., Martin, W., Kallianpur, A., Chung, M. K., Jehi, L., Sharifi, N., Erzurum, S., Eng, C., and Cheng, F. (2020). New insights into genetic susceptibility of COVID-19: an ACE2 and TMPRSS2 polymorphism analysis. BMC Med 18, 216.
Huang, C., Wang, Y., Li, X., Ren, L., Zhao, J., Hu, Y., Zhang, L., Fan, G., Xu, J., Gu, X., el al. (2020). Clinical features of patients infected with 2019 novel coronavirus in Wuhan, China. Lancet 395, 497-506.
Hussain, M., Jabeen, N., Raza, F., Shabbir, S., Baig, A. A., Amanullah, A., and Aziz, B. (2020). Structural variations in human ACE2 may influence its binding with SARS-CoV-2 spike protein. J Med Virol.
Ikram, M. A., Brusselle, G. G. O., Murad, S. D., van Duijn, C. M., Franco, O. H., Goedegebure, A., Klaver, C. C. W., Nijsten, T. E. C., Peeters, R. P., Stricker, B. H., et al. (2017). The Rotterdam Study: 2018 update on objectives, design and main results. Eur J Epidemiol 32, 807-850.
International HapMap, C., Frazer, K. A., Ballinger, D. G., Cox, D. R., Hinds, D. A., Stuve, L. L., Gibbs, R. A., Belmont, J. W., Boudreau, A., Hardenbol, P., et al. (2007). A second generation human haplotype map of over 3.1 million SNPs. Nature 449, 851-861.
JHU (2020). https://coronavirus.ihu.edu/.
Jiang, S. (2020). Don't rush to deploy COVID-19 vaccines and drugs without sufficient safety guarantees. Nature 579, 321.
Kachuri, L., Francis, S. S., Morrison, M., Wendt, G., Bossé, Y., Cavazos, T. B., Rashkin, S. R., Ziv, E., and Witte, J. S. (2020). The landscape of host genetic factors involved in immune response to common viral infections. medRxiv, 2020.2005.2001.20088054.
Karczewski, K. J., Francioli, L. C., Tiao, G., Cummings, B. B., Alfóldi, J., Wang, Q., Collins, R. L., Laricchia, K. M., Ganna, A., Birnbaum, D. P., et al. (2019). Variation across 141,456 human exomes and genomes reveals the spectrum of loss-of-function intolerance across human protein-coding genes. bioRxiv doi.org/10.1101/531210.
Kruse, R. L. (2020). Therapeutic strategies in an outbreak scenario to treat the novel coronavirus originating in Wuhan, China. F1000Res 9, 72.
Lei, C., Fu, W., Qian, K., Li, T., Zhang, S., Ding, M., and Hu, S. (2020). Potent neutralization of 2019 novel coronavirus by recombinant ACE2-Ig. bioRxiv doi.org/10.1101/2020.02.01.929976.
Letko, M., Marzi, A., and Munster, V. (2020). Functional assessment of cell entry and receptor usage for SARS-CoV-2 and other lineage B betacoronaviruses. Nat Microbiol 5, 562-569.
Li, F. (2008). Structural analysis of major species barriers between humans and palm civets for severe acute respiratory syndrome coronavirus infections. J Virol 82, 6984-6991.
Li, F. (2016). Structure, Function, and Evolution of Coronavirus Spike Proteins. Annu Rev Virol 3, 237-261.
Li, F., Li, W., Farzan, M., and Harrison, S. C. (2005a). Structure of SARS coronavirus spike receptor-binding domain complexed with receptor. Science 309, 1864-1868.
Li, G., and De Clercq, E. (2020). Therapeutic options for the 2019 novel coronavirus (2019-nCoV). Nat Rev Drug Discov 19, 149-150.
Li, W., Zhang, C., Sui, J., Kuhn, J. H., Moore, M. J., Luo, S., Wong, S. K., Huang, I. C., Xu, K., Vasilieva, N., el al. (2005b). Receptor and viral determinants of SARS-coronavirus adaptation to human ACE2. EMBO J 24, 1634-1643.
Lin, H. X., Feng, Y., Wong, G., Wang, L., Li, B., Zhao, X., Li, Y., Smaill, F., and Zhang, C. (2008). Identification of residues in the receptor-binding domain (RBD) of the spike protein of human coronavirus NL63 that are critical for the RBD-ACE2 receptor interaction. J Gen Virol 89, 1015-1024.
Lu, J., and Sun, P. D. (2020). High affinity binding of SARS-CoV-2 spike protein enhances ACE2 carboxypeptidase activity. bioRxiv.
Lui, I., Zhou, X. X., Lim, S. A., Elledge, S. K., Solomon, P., Rettko, N. J., Zha, B. S., Kirkemo, L. L., Gramespacher, J. A., Liu, J., et al. (2020). Trimeric SARS-CoV-2 Spike interacts with dimeric ACE2 with limited intra-Spike avidity. bioRxiv, 2020.2005.2021.109157.
McLaren, W., Gil, L., Hunt, S. E., Riat, H. S., Ritchie, G. R., Thormann, A., Flicek, P., and Cunningham, F. (2016). The Ensembl Variant Effect Predictor. Genome Biol 17, 122.
Ou, X., Liu, Y., Lei, X., Li, P., Mi, D., Ren, L., Guo, L., Guo, R., Chen, T., Hu, J., et al. (2020). Characterization of spike glycoprotein of SARS-CoV-2 on virus entry and its immune cross-reactivity with SARS-CoV. Nat Commun 11, 1620.
Procko, E. (2020). The sequence of human ACE2 is suboptimal for binding the S spike protein of SARS coronavirus 2. bioRxiv https://doi.org/10.1101/2020.1103.1116.994236.
Shang, J., Ye, G., Shi, K., Wan, Y., Luo, C., Aihara, H., Geng, Q., Auerbach, A., and Li, F. (2020). Structural basis of receptor recognition by SARS-CoV-2. Nature, https://www.nature.com/articles/s42586-41020-42179-y.
Simmons, G., Gosalia, D. N., Rennekamp, A. J., Reeves, J. D., Diamond, S. L., and Bates, P. (2005). Inhibitors of cathepsin L prevent severe acute respiratory syndrome coronavirus entry. Proc Natl Acad Sci USA 102, 11876-11881.
Suryamohan, K., Krishnankutty, S. P., Guillory, J., Jevit, M., Schroder, M. S., Wu, M., Kuriakose, B., Mathew, O. K., Perumal, R. C., Koludarov, I., et al. (2020). The Indian cobra reference genome and transcriptome enables comprehensive identification of venom toxins. Nat Genet 52, 106-117.
Tadaka, S., Katsuoka, F., Ueki, M., Kojima, K., Makino, S., Saito, S., Otsuki, A., Gocho, C., Sakurai-Yageta, M., Danjoh, I., et al. (2019). 3.5KJPNv2: an allele frequency panel of 3552 Japanese individuals including the X chromosome. Hum Genome Var 6, 28.
Walls, A. C., Park, Y. J., Tortorici, M. A., Wall, A., McGuire, A. T., and Veesler, D. (2020). Structure, Function, and Antigenicity of the SARS-CoV-2 Spike Glycoprotein. Cell, 10.1016/j.cell.2020.1002.1058.
Walls, A. C., Xiong, X., Park, Y. J., Tortorici, M. A., Snijder, J., Quispe, J., Cameroni, E., Gopal, R., Dai, M., Lanzavecchia, A., et al. (2019). Unexpected Receptor Functional Mimicry Elucidates Activation of Coronavirus Fusion. Cell 176, 1026-1039 e1015.
Wan, Y., Shang, J., Graham, R., Baric, R. S., and Li, F. (2020). Receptor Recognition by the Novel Coronavirus from Wuhan: an Analysis Based on Decade-Long Structural Studies of SARS Coronavirus. J Virol 94.
Wong, S. K., Li, W., Moore, M. J., Choe, H., and Farzan, M. (2004). A 193-amino acid fragment of the SARS coronavirus S protein efficiently binds angiotensin-converting enzyme 2. J Biol Chem 279, 3197-3201.
Wrapp, D., Wang, N., Corbett, K. S., Goldsmith, J. A., Hsieh, C. L., Abiona, O., Graham, B. S., and McLellan, J. S. (2020). Cryo-EM structure of the 2019-nCoV spike in the prefusion conformation. Science 367, 1260-1263.
Wu, K., Peng, G., Wilken, M., Geraghty, R. J., and Li, F. (2012). Mechanisms of host receptor adaptation by severe acute respiratory syndrome coronavirus. J Biol Chem 287, 8904-8911.
Yan, R., Zhang, Y., Li, Y., Xia, L., Guo, Y., and Zhou, Q. (2020). Structural basis for the recognition of SARS-CoV-2 by full-length human ACE2. Science 367, 1444-1448.
Yuen, K. S., Ye, Z. W., Fung, S. Y., Chan, C. P., and Jin, D. Y. (2020). SARS-CoV-2 and COVID-19: The most important research questions. Cell Biosci 10, 40.
Zaki, A. M., van Boheemen, S., Bestebroer, T. M., Osterhaus, A. D., and Fouchier, R. A. (2012). Isolation of a novel coronavirus from a man with pneumonia in Saudi Arabia. N Engl J Med 367, 1814-1820.
Zhou, P., Yang, X. L., Wang, X. G., Hu, B., Zhang, L., Zhang, W., Si, H. R., Zhu, Y., Li, B., Huang, C. L., et al. (2020a). A pneumonia outbreak associated with a new coronavirus of probable bat origin. Nature 579, 270-273.
Zhou, Y., Hou, Y., Shen, J., Huang, Y., Martin, W., and Cheng, F. (2020b). Network-based drug repurposing for novel coronavirus 2019-nCoV/SARS-CoV-2. Cell Discov 6, 14.
Zhu, N., Zhang, D., Wang, W., Li, X., Yang, B., Song, J., Zhao, X., Huang, B., Shi, W., Lu, R., et al. (2020). A Novel Coronavirus from Patients with Pneumonia in China, 2019. N Engl J Med 382, 727-733.
Zoufaly, A., Poglitsch, M., Aberle, J. H., Hoepler, W., Seitz, T., Traugott, M., Grieb, A., Pawelka, E., Laferl, H., Wenisch, C., et al. (2020). Human recombinant soluble ACE2 in severe COVID-19. Lancet Respir Med 8, 1154-1158.

1. An isolated SARS-CoV-2 binding protein complex comprising an extracellular domain or fragment thereof of an angiotensin converting enzyme 2 (ACE2) protein or its variant joined to a non-ACT 2 molecule or compound.

2. The isolated SARS-CoV-2 binding protein complex of claim 1, wherein the non ACE2 compound is a biological entity.

3. The isolated SARS-CoV-2 binding protein complex of claim 2, wherein the biological entity is selected from a group consisting of a protein, polypeptide or peptide, albumin.

4. The isolated SARS-CoV-2 binding protein complex of claim 3, wherein the protein is an immunoglobulin molecule or antibody molecule or variant or fragment thereof.

5. The isolated SARS-CoV-2 binding protein complex of claim 4, wherein the antibody fragment is a Fc.

6. The isolated SARS-CoV-2 binding protein complex of claim 4, wherein the antibody fragment is selected from the group consisting of Fab, Fab′, F(ab)′, scFv, and F(ab)′₂.

7. The isolated SARS-CoV-2 binding protein complex of claim 4, wherein the antibody recognizes and binds a SARS-CoV-2.

8. (canceled)

9. (canceled)

10. (canceled)

11. (canceled)

12. (canceled)

13. (canceled)

14. (canceled)

15. (canceled)

16. The isolated SARS-CoV-2 binding protein complex of claim 1, wherein the extracellular domain of the ACE2 protein comprises or consists of the amino acid sequences between a signal sequence and a transmembrane domain of the ACE2 protein but lacks a signal sequence, transmembrane domain and cytosolic domain.

17. The isolated SARS-CoV-2 binding protein complex of claim 1, wherein the extracellular domain of the ACE2 protein consists of or comprises a peptidase domain and collectrin domain.

18. The isolated SARS-CoV-2 binding protein complex of claim 17, wherein the extracellular domain encompasses amino acid residues 18 to 740 of sequence provided in FIG. 4 or SEQ 1) NO: 1 (UniProtKB ID: Q9BYF1-1) as shown below:
(SEQ ID NO: 2)
QSTIEEQAKTFLDKFNHEAEDLFYQSSLASWNYNTNITEENVQNMNNAGD
KWSAFLKEQSTLAQMYPLQEIQNLTVKLQLQALQQNGSSVLSEDKSKRLN
TILNTMSTIYSTGKVCNPDNPQECLLLEPGLNEIMANSLDYNERLWAWES
WPSEVDKQLRPLYEEYVVLKNEMARANHYEDYGDYWRGDYEVNGVDGYDY
DRGQLIEDVEHTFEEIKPLYEHLHAYVPAKLMNAYPSYISPIGCLPAHLL
GDMWGRFWTNLYSLTVPFGQKPNIDVTDAMVQQAWDAQRIFKEAEKFFVS
VGLPNMTQGFWENSMLTDPGNVQKAVCHPTAWDLGKGDFRILMCTKVTMD
DFLTAHHEMGHIQYDMAYAAQPFLLRNGANEGFHEAVGEIMSLSAATPKH
LKSIGLLSPDFQEDNETEINFLLKQALTIVGTLPFTYMLEKWRWMVFKGE
IPKDQWMKKWWEMKREIVGVVEPVPHDETYCDPASLFHVSNDYSFIRYYT
RTLYQFQFQEALCQAAKHEGPLHKCDISNSTEAGQKLFNMLRLGKSEPWT
LALENVVGAKNMNVRPLLNYFEPLFTWLKDQNKNSFVGWSTDWSPYADQS
IKVRISLKSALGDKAYEWNDNEMYLFRSSVAYAMRQYFLKVKNQMILFGE
EDVRVANLKPRISFNFFVTAPKNVSDIIPRTEVEHAIRMSRSRINDAFRL
NDNSLEFLGIQFTLGPPNQPPVS
or a variant thereof.

19. The isolated SARS-CoV-2 binding protein complex of claim 1, wherein the ACE2 variant has at least one amino acid change from a reference full length ACE2 protein as provided in SEQ ID NO: 1.

20. The isolated SARS-CoV-2 binding protein complex of claim 1), wherein the amino acid change increases binding or binding affinity of the extracellular domain or fragment thereof for a SARS-CoV-2 virus or a SARS-CoV-2 spike glycoprotein (S-protein).

21. (canceled)

22. The isolated SARS-CoV-2 binding protein complex of claim 1, wherein the ACE2 variant has at least two amino acid changes from a reference full length ACE2 protein as provided in SEQ ID NO: 1.

23. The isolated SARS-CoV-2 binding protein complex of claim 1, wherein the ACE2 variant has at least three amino acid changes from a reference full length ACE2 protein as provided in SEQ ID NO: 1.

24. The isolated SARS-CoV-2 binding protein complex of claim 19, wherein the amino acid change(s) increases binding or binding affinity of the ACE2 variant for SARS-CoV-2 virus, SARS-CoV-2 S-protein, CoV-2-S-RB) comprising amino acids 319-541 of NCBI Reference Sequence Accession Number YP_009724390.1, SARS-CoV-2 Spike-protein S1 subunit comprising amino acids 16-681 of NCBI Reference Sequence Accession number YP_009724390.1 and/or SARS-CoV-2 S-protein trimer.

25. The isolated SARS-CoV-2 binding protein complex of claim 24, wherein the SARS-CoV-2 S-protein trimer comprises a SARS-CoV-2 ectodomain and a T4 fibritin trimerization motif.

26. The isolated SARS-CoV-2 binding protein complex of claim 24, wherein the SARS-CoV-2 ectodomain comprises amino acids 1-1208 of NCBI Reference Number YP_009724390.1 or variant thereof.

27. The isolated SARS-CoV-2 binding protein complex of claim 26, wherein the variant of the SARS-CoV-2 ectodomain comprises one or more amino acid substitutions selected from the group consisting of K986P, V987P, RRAR to GSAS (residues 682-685) at a furin-cleavage site or a combination thereof.

28. The isolated SARS-CoV-2 binding protein complex of claim 26, wherein the variant of the SARS-CoV-2 ectodomain comprises the following amino acid substitutions: K986P, V987P and RRAR to GSAS (residues 682-685) at a furin-cleavage site.

29-406. (canceled)
